SEQUENCE LISTING (343 sequences)

SEQ ID NO: 1 - protein, 7 aa, Murine
TSGMSVG

SEQ ID NO: 2 - protein, 16 aa, Murine
DIWWDDKKDYNPSLKS

SEQ ID NO: 3 - protein, 10 aa, Murine
SMITNWYFDV

SEQ ID NO: 4 - protein, 10 aa, Murine
KCQLSVGYMH

SEQ ID NO: 5 - protein, 7 aa, Murine
DTSKLAS

SEQ ID NO: 6 - protein, 9 aa, Murine
FQGSGYPFT

SEQ ID NO: 7 - protein, 120 aa, Artificial Sequence
  Humanized antibody - VH Domain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 8 - protein, 106 aa, Artificial Sequence
  misc_feature: Humanized antibody - VL Domain
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 9 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARSMITNFYFDVWGQGTTVTVSS

SEQ ID NO: 10 - protein, 7 aa, Artificial Sequence
  Amino acid sequence derived from Murine monoclonal antibody and further modified by amino acid substitutions
TAGMSVG

SEQ ID NO: 11 - protein, 106 aa, Artificial Sequence [sequence omitted]

SEQ ID NO: 12 - protein, 10 aa, Artificial Sequence
  Amino acid sequence derived from Murine monoclonal antibody and further modified by amino acid substitutions
SMITNFYFDV

SEQ ID NO: 13 - protein, 106 aa, Artificial Sequence
  Humanized antibody - VL Domain
DIQMTQSPSTLSASVGDRVTITCSASSSVGYMHWYQQKPGKAPKLLIYDTFKLASGVPSRFSGSGSGTEFTLTISSLQPDDFATYYCFQFSGYPFTFGGGTKVEIK

SEQ ID NO: 14 - protein, 10 aa, Artificial Sequence
  Amino acid sequence derived from Murine monoclonal antibody and further modified by amino acid substitutions
SASSSVGYMH

SEQ ID NO: 15 - protein, 7 aa, Artificial Sequence
  Amino acid sequence derived from Murine monoclonal antibody and further modified by amino acid substitutions
DTFKLAS

SEQ ID NO: 16 - protein, 9 aa, Artificial Sequence
  Amino acid sequence derived from Murine monoclonal antibody and further modified by amino acid substitutions
FQFSGYPFT

SEQ ID NO: 17 - protein, 120 aa, Artificial Sequence
  Humanized antibody - VH Domain
QVTLRESGPALVKPTQTLTLTCTFSGFSLSTPGMSVGWIRQPPGKALEWLADIWWDDKKHYNPSLKDRLTISKDTSKNQVVLKVTNMDPADTATYYCARDMIFNFYFDVWGQGTTVTVSS

SEQ ID NO: 18 - protein, 7 aa, Artificial Sequence
  Amino acid sequence derived from Murine monoclonal antibody and further modified by amino acid substitutions
TPGMSVG

SEQ ID NO: 19 - protein, 16 aa, Artificial Sequence
  Amino acid sequence derived from Murine monoclonal antibody and further modified by amino acid substitutions
DIWWDDKKHYNPSLKD

SEQ ID NO: 20 - protein, 10 aa, Artificial Sequence
  Amino acid sequence derived from Murine monoclonal antibody and further modified by amino acid substitutions
DMIFNFYFDV

SEQ ID NO: 21 - protein, 106 aa, Artificial Sequence
  Humanized antibody - VL Domain
DIQMTQSPSTLSASVGDRVTITCSLSSRVGYMHWYQQKPGKAPKLLIYDTFYLSSGVPSRFSGSGSGTEFTLTISSLQPDDFATYYCFQGSGYPFTFGGGTKVEIK

SEQ ID NO: 22 - protein, 10 aa, Artificial Sequence
  Amino acid sequence derived from Murine monoclonal antibody and further modified by amino acid substitutions
SLSSRVGYMH

SEQ ID NO: 23 - protein, 7 aa, Artificial Sequence
  Amino acid sequence derived from Murine monoclonal antibody and further modified by amino acid substitutions
DTFYLSS

SEQ ID NO: 24 - protein, 120 aa, Artificial Sequence
  Humanized antibody - VH Domain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNFYFDVWGQGTTVTVSS

SEQ ID NO: 25 - protein, 16 aa, Artificial Sequence
  Amino acid sequence derived from Murine monoclonal antibody and further modified by amino acid substitutions
DIWWDGKKHYNPSLKD

SEQ ID NO: 26 - protein, 106 aa, Artificial Sequence
  Humanized antibody - VL Domain
DIQMTQSPSTLSASVGDRVTITCSLSSRVGYMHWYQQKPGKAPKLLIYDTRGLPSGVPSRFSGSGSGTEFTLTISSLQPDDFATYYCFQGSGYPFTFGGGTKVEIK

SEQ ID NO: 27 - protein, 7 aa, Artificial Sequence
  Amino acid sequence derived from Murine monoclonal antibody and further modified by amino acid substitutions
DTRGLPS

SEQ ID NO: 28 - protein, 120 aa, Artificial Sequence
  Humanized antibody - VH Domain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNWYFDVWGQGTTVTVSS

SEQ ID NO: 29 - protein, 10 aa, Artificial Sequence
  Amino acid sequence derived from Murine monoclonal antibody and further modified by amino acid substitutions
DMIFNWYFDV

SEQ ID NO: 30 - protein, 106 aa, Artificial Sequence
  Humanized antibody - VL Domain
DIQMTQSPSTLSASVGDRVTITCSPSSRVGYMHWYQQKPGKAPKLLIYDTMRLASGVPSRFSGSGSGTEFTLTISSLQPDDFATYYCFQGSGYPFTFGGGTKVEIK

SEQ ID NO: 31 - protein, 10 aa, Artificial Sequence
  Amino acid sequence derived from Murine monoclonal antibody and further modified by amino acid substitutions
SPSSRVGYMH

SEQ ID NO: 32 - protein, 7 aa, Artificial Sequence
  Amino acid sequence derived from Murine monoclonal antibody and further modified by amino acid substitutions
DTMRLAS

SEQ ID NO: 33 - protein, 120 aa, Artificial Sequence
  Humanized antibody - VH Domain
QVTLRESGPALVKPTQTLTLTCTFSGFSLSTAGMSVGWIRQPPGKALEWLADIWWDGKKHYNPSLKDRLTISKDTSKNQVVLKVTNMDPADTATYYCARDMIFNWYFDVWGQGTTVTVSS

SEQ ID NO: 34 - protein, 106 aa, Artificial Sequence
  Humanized antibody - VL Domain
DIQMTQSPSTLSASVGDRVTITCSLSSRVGYMHWYQQKPGKAPKLLIYDTFKLSSGVPSRFSGSGSGTEFTLTISSLQPDDFATYYCFQGSGYPFTFGGGTKVEIK

SEQ ID NO: 35 - protein, 7 aa, Artificial Sequence
  Amino acid sequence derived from Murine monoclonal antibody and further modified by amino acid substitutions
DTFKLSS

SEQ ID NO: 36 - protein, 120 aa, Artificial Sequence
  Humanized antibody - VH Domain
QVTLRESGPALVKPTQTLTLTCTFSGFSLSTAGMSVGWIRQPPGKALEWLADIWWDGKKDYNPSLKDRLTISKDTSKNQVVLKVTNMDPADTATYYCARDMIFNFYFDVWGQGTTVTVSS

SEQ ID NO: 37 - protein, 16 aa, Artificial Sequence
  Amino acid sequence derived from Murine monoclonal antibody and further modified by amino acid substitutions
DIWWDGKKDYNPSLKD

SEQ ID NO: 38 - protein, 106 aa, Artificial Sequence
  Humanized antibody - VL Domain
DIQMTQSPSTLSASVGDRVTITCSASSRVGYMHWYQQKPGKAPKLLIYDTFKLSSGVPSRFSGSGSGTEFTLTISSLQPDDFATYYCFQGSGYPFTFGGGTKVEIK

SEQ ID NO: 39 - protein, 10 aa, Artificial Sequence
  Amino acid sequence derived from Murine monoclonal antibody and further modified by amino acid substitutions
SASSRVGYMH

SEQ ID NO: 40 - protein, 120 aa, Artificial Sequence
  Humanized antibody - VH Domain
QVTLRESGPALVKPTQTLTLTCTFSGFSLSTAGMSVGWIRQPPGKALEWLADIWWDGKKSYNPSLKDRLTISKDTSKNQVVLKVTNMDPADTATYYCARDMIFNFYFDVWGQGTTVTVSS

SEQ ID NO: 41 - protein, 16 aa, Artificial Sequence
  Amino acid sequence derived from Murine monoclonal antibody and further modified by amino acid substitutions
DIWWDGKKSYNPSLKD

SEQ ID NO: 42 - protein, 106 aa, Artificial Sequence
  Humanized antibody - VL Domain
DIQMTQSPSTLSASVGDRVTITCSLSSRVGYMHWYQQKPGKAPKLLIYDTMYQSSGVPSRFSGSGSGTEFTLTISSLQPDDFATYYCFQGSGYPFTFGGGTKVEIK

SEQ ID NO: 43 - protein, 7 aa, Artificial Sequence
  Amino acid sequence derived from Murine monoclonal antibody and further modified by amino acid substitutions
DTMYQSS

SEQ ID NO: 44 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNFYFDVWGQGTTVTVSS

SEQ ID NO: 45 - protein, 16 aa, Artificial Sequence
  Amino acid sequence derived from Murine monoclonal antibody and further modified by amino acid substitutions
DIWWDDKKSYNPSLKD

SEQ ID NO: 46 - protein, 106 aa, Artificial Sequence
  Humanized antibody - VL Domain
DIQMTQSPSTLSASVGDRVTITCLPSSRVGYMHWYQQKPGKAPKLLIYDTMYQSSGVPSRFSGSGSGTEFTLTISSLQPDDFATYYCFQGSGYPFTFGGGTKVEIK

SEQ ID NO: 47 - protein, 10 aa, Artificial Sequence
  Amino acid sequence derived from Murine monoclonal antibody and further modified by amino acid substitutions
LPSSRVGYMH

SEQ ID NO: 48 - protein, 120 aa, Artificial Sequence [sequence omitted]

SEQ ID NO: 49 - protein, 106 aa, Artificial Sequence
  Humanized antibody - VL Domain
DIQMTQSPSTLSASVGDRVTITCSASSRVGYMHWYQQKPGKAPKLLIYDTFFLDSGVPSRFSGSGSGTEFTLTISSLQPDDFATYYCFQGSGYPFTFGGGTKVEIK

SEQ ID NO: 50 - protein, 7 aa, Artificial Sequence
  Amino acid sequence derived from Murine monoclonal antibody and further modified by amino acid substitutions
DTFFLDS

SEQ ID NO: 51 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNWYFDVWGQGTTVTVSS

SEQ ID NO: 52 - protein, 106 aa, Artificial Sequence
  Humanized antibody - VL Domain
DIQMTQSPSTLSASVGDRVTITCSPSSRVGYMHWYQQKPGKAPKLLIYDTRYQSSGVPSRFSGSGSGTEFTLTISSLQPDDFATYYCFQGSGYPFTFGGGTKVEIK

SEQ ID NO: 53 - protein, 7 aa, Artificial Sequence
  Amino acid sequence derived from Murine monoclonal antibody and further modified by amino acid substitutions
DTRYQSS

SEQ ID NO: 54 - protein, 106 aa, Artificial Sequence
  Humanized antibody - VL Domain
DIQMTQSPSTLSASVGDRVTITCSASSSVGYMHWYQQKPGKAPKLLIYDTSKLASGVPSRFSGSGSGTEFTLTISSLQPDDFATYYCFQGSGYPFTFGGGTKVEIK

SEQ ID NO: 55 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMIFNWYFDVWGQGTTVTVSS

SEQ ID NO: 56 - protein, 106 aa, Artificial Sequence
  Humanized antibody - VL Domain
DIQMTQSPSTLSASVGDRVTITCSASSSVGYMHWYQQKPGKAPKLLIYDTFKLASGVPSRFSGSGSGTEFTLTISSLQPDDFATYYCFQGSGYPFTFGGGTKVEIK

SEQ ID NO: 57 - protein, 7 aa, Artificial Sequence
  Amino acid sequence derived from Murine monoclonal antibody and further modified by amino acid substitutions
DTYKQTS

SEQ ID NO: 58 - protein, 106 aa, Artificial Sequence
  Humanized antibody - VL Domain
DIQMTQSPSTLSASVGDRVTITCSASSSVGYMHWYQQKPGKAPKLLIYDTRYLSSGVPSRFSGSGSGTEFTLTISSLQPDDFATYYCFQGSGYPFTFGGGTKVEIK

SEQ ID NO: 59 - protein, 7 aa, Artificial Sequence
  Amino acid sequence derived from Murine monoclonal antibody and further modified by amino acid substitutions
DTRYLSS

SEQ ID NO: 60 - protein, 106 aa, Artificial Sequence
  Humanized antibody - VL Domain
DIQMTQSPSTLSASVGDRVTITCSASSSVGYMHWYQQKPGKAPKLLIYDTFKLASGVPSRFSGSGSGTEFTLTISSLQPDDFATYYCFQGSFYPFTFGGGTKVEIK

SEQ ID NO: 61 - protein, 9 aa, Artificial Sequence
  Amino acid sequence derived from Murine monoclonal antibody and further modified by amino acid substitutions
FQGSFYPFT

SEQ ID NO: 62 - protein, 106 aa, Artificial Sequence
  Humanized antibody - VL Domain
DIQMTQSPSTLSASVGDRVTITCSASSSVGYMHWYQQKPGKAPKLLIYDTFKLTSGVPSRFSGSGSGTEFTLTISSLQPDDFATYYCFQGSGYPFTFGGGTKVEIK

SEQ ID NO: 63 - protein, 7 aa, Artificial Sequence
  Amino acid sequence derived from Murine monoclonal antibody and further modified by amino acid substitutions
DTFKLTS

SEQ ID NO: 64 - protein, 106 aa, Artificial Sequence
  Humanized antibody - VL Domain
DIQMTQSPSTLSASVGDRVTITCSASSRVGYMHWYQQKPGKAPKLLIYDTFKLASGVPSRFSGSGSGTEFTLTISSLQPDDFATYYCFQGSGYPFTFGGGTKVEIK

SEQ ID NO: 65 - protein, 106 aa, Artificial Sequence
  Humanized antibody - VL Domain
DIQMTQSPSTLSASVGDRVTITCSASSSVGYMHWYQQKPGKAPKLLIYDTFRLASGVPSRFSGSGSGTEFTLTISSLQPDDFATYYCFQGSGYPFTFGGGTKVEIK

SEQ ID NO: 66 - protein, 7 aa, Artificial Sequence
  Amino acid sequence derived from Murine monoclonal antibody and further modified by amino acid substitutions
DTFRLAS

SEQ ID NO: 67 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNWYFDVWGQGTTVTVSS

SEQ ID NO: 68 - protein, 106 aa, Artificial Sequence
  Humanized antibody - VL Domain
DIQMTQSPSTLSASVGDRVTITCSPSSRVGYMHWYQQKPGKAPKLLIYDTYRHSSGVPSRFSGSGSGTEFTLTISSLQPDDFATYYCFQGSGYPFTFGGGTKVEIK

SEQ ID NO: 69 - protein, 7 aa, Artificial Sequence
  Amino acid sequence derived from Murine monoclonal antibody and further modified by amino acid substitutions
DTYRHSS

SEQ ID NO: 70 - protein, 106 aa, Artificial Sequence
  Humanized antibody - VL Domain
DIQMTQSPSTLSASVGDRVTITCSASSSVGYMHWYQQKPGKAPKLLIYDTYKQTSGVPSRFSGSGSGTEFTLTISSLQPDDFATYYCFQGSGYPFTFGGGTKVEIK

SEQ ID NO: 71 - protein, 106 aa, Artificial Sequence
  Humanized antibody - VL Domain
DIQMTQSPSTLSASVGDRVTITCSLSSSVGYMHWYQQKPGKAPKLLIYDTFFHRSGVPSRFSGSGSGTEFTLTISSLQPDDFATYYCFQGSGYPFTFGGGTKVEIK

SEQ ID NO: 72 - protein, 10 aa, Artificial Sequence
  Amino acid sequence derived from Murine monoclonal antibody and further modified by amino acid substitutions
SLSSSVGYMH

SEQ ID NO: 73 - protein, 7 aa, Artificial Sequence
  Amino acid sequence derived from Murine monoclonal antibody and further modified by amino acid substitutions
DTFFHRS

SEQ ID NO: 74 - protein, 106 aa, Artificial Sequence
  Humanized antibody - VL Domain
DIQMTQSPSTLSASVGDRVTITCSASSRVGYMHWYQQKPGKAPKLLIYDTLLLDSGVPSRFSGSGSGTEFTLTISSLQPDDFATYYCFQGSGYPFTFGGGTKVEIK

SEQ ID NO: 75 - protein, 7 aa, Artificial Sequence
  Amino acid sequence derived from Murine monoclonal antibody and further modified by amino acid substitutions
DTLLLDS

SEQ ID NO: 76 - protein, 106 aa, Artificial Sequence
  Humanized antibody - VL Domain
DIQMTQSPSTLSASVGDRVTITCSASSRVGYMHWYQQKPGKAPKLLIYDTSFLDSGVPSRFSGSGSGTEFTLTISSLQPDDFATYYCFQGSGYPFTFGGGTKVEIK

SEQ ID NO: 77 - protein, 7 aa, Artificial Sequence
  Amino acid sequence derived from Murine monoclonal antibody and further modified by amino acid substitutions
DTSFLDS

SEQ ID NO: 78 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMITNFYFDVWGQGTTVTVSS

SEQ ID NO: 79 - protein, 10 aa, Artificial Sequence
  Amino acid sequence derived from Murine monoclonal antibody and further modified by amino acid substitutions
DMITNFYFDV

SEQ ID NO: 80 - protein, 10 aa, Artificial Sequence
  Amino acid sequence derived from Murine monoclonal antibody and further modified by amino acid substitutions
KCQSSVGYMH

SEQ ID NO: 81 - protein, 7 aa, Artificial Sequence
  Amino acid sequence derived from Murine monoclonal antibody and further modified by amino acid substitutions
DTSYLAS

SEQ ID NO: 82 - protein, 16 aa, Artificial Sequence
  Amino acid sequence derived from Murine monoclonal antibody and further modified by amino acid substitutions
DIWWDDKKHYNPSLKS

SEQ ID NO: 83 - protein, 10 aa, Artificial Sequence
  Amino acid sequence derived from Murine monoclonal antibody and further modified by amino acid substitutions
DMITNWYFDV

SEQ ID NO: 84 - protein, 10 aa, Artificial Sequence
  Amino acid sequence derived from Murine monoclonal antibody and further modified by amino acid substitutions
KCQSRVGYMH

SEQ ID NO: 85 - protein, 7 aa, Artificial Sequence
  Amino acid sequence derived from Murine monoclonal antibody and further modified by amino acid substitutions
DTSYLSS

SEQ ID NO: 86 - protein, 16 aa, Artificial Sequence
  Amino acid sequence derived from Murine monoclonal antibody and further modified by amino acid substitutions
DIWWDDKKDYNPSLKD

SEQ ID NO: 87 - protein, 10 aa, Artificial Sequence
  Amino acid sequence derived from Murine monoclonal antibody and further modified by amino acid substitutions
KCQLRVGYMH

SEQ ID NO: 88 - protein, 7 aa, Artificial Sequence
  Amino acid sequence derived from Murine monoclonal antibody and further modified by amino acid substitutions
DTKKLSS

SEQ ID NO: 89 - protein, 10 aa, Artificial Sequence
  Amino acid sequence derived from Murine monoclonal antibody and further modified by amino acid substitutions
KLQLSVGYMH

SEQ ID NO: 90 - protein, 7 aa, Artificial Sequence
  Amino acid sequence derived from Murine monoclonal antibody and further modified by amino acid substitutions
DTFYLSS

SEQ ID NO: 91 - protein, 16 aa, Artificial Sequence
  Amino acid sequence derived from Murine monoclonal antibody and further modified by amino acid substitutions
DIWWDDKKHYNPSLKS

SEQ ID NO: 92 - protein, 10 aa, Artificial Sequence
  Amino acid sequence derived from Murine monoclonal antibody and further modified by amino acid substitutions
KLQSSVGYMH

SEQ ID NO: 93 - protein, 16 aa, Artificial Sequence
  Amino acid sequence derived from Murine monoclonal antibody and further modified by amino acid substitutions
DIWWDDKKHYNPSLKS

SEQ ID NO: 94 - protein, 10 aa, Artificial Sequence
  Amino acid sequence derived from Murine monoclonal antibody and further modified by amino acid substitutions
SMIFNWYFDV

SEQ ID NO: 95 - protein, 10 aa, Artificial Sequence
  Amino acid sequence derived from Murine monoclonal antibody and further modified by amino acid substitutions
KLQSRVGYMH

SEQ ID NO: 96 - protein, 7 aa, Artificial Sequence
  Amino acid sequence derived from Murine monoclonal antibody and further modified by amino acid substitutions
DTFKLSS

SEQ ID NO: 97 - protein, 10 aa, Artificial Sequence
  Amino acid sequence derived from Murine monoclonal antibody and further modified by amino acid substitutions
SMIFNFYFDV

SEQ ID NO: 98 - protein, 10 aa, Artificial Sequence
  Amino acid sequence derived from Murine monoclonal antibody and further modified by amino acid substitutions
KLQLRVGYMH

SEQ ID NO: 99 - protein, 7 aa, Artificial Sequence
  Amino acid sequence derived from Murine monoclonal antibody and further modified by amino acid substitutions
DTFYLAS

SEQ ID NO: 100 - protein, 16 aa, Artificial Sequence
  Amino acid sequence derived from Murine monoclonal antibody and further modified by amino acid substitutions
DIWWDGKKDYNPSLKS

SEQ ID NO: 101 - protein, 10 aa, Artificial Sequence
  Amino acid sequence derived from Murine monoclonal antibody and further modified by amino acid substitutions
KLSLSVGYMH

SEQ ID NO: 102 - protein, 7 aa, Artificial Sequence
  Amino acid sequence derived from Murine monoclonal antibody and further modified by amino acid substitutions
DTSKLPS

SEQ ID NO: 103 - protein, 16 aa, Artificial Sequence
  Amino acid sequence derived from Murine monoclonal antibody and further modified by amino acid substitutions
DIWWDGKKDYNPSLKD

SEQ ID NO: 104 - protein, 10 aa, Artificial Sequence
  Amino acid sequence derived from Murine monoclonal antibody and further modified by amino acid substitutions
KLSSSVGYMH

SEQ ID NO: 105 - protein, 7 aa, Artificial Sequence
  Amino acid sequence derived from Murine monoclonal antibody and further modified by amino acid substitutions
DTSGLAS

SEQ ID NO: 106 - protein, 16 aa, Artificial Sequence
  Amino acid sequence derived from Murine monoclonal antibody and further modified by amino acid substitutions
DIWWDGKKHYNPSLKS

SEQ ID NO: 107 - protein, 10 aa, Artificial Sequence
  Amino acid sequence derived from Murine monoclonal antibody and further modified by amino acid substitutions
KLSSRVGYMH

SEQ ID NO: 108 - protein, 7 aa, Artificial Sequence
  Amino acid sequence derived from Murine monoclonal antibody and further modified by amino acid substitutions
DTSGLPS

SEQ ID NO: 109 - protein, 16 aa, Artificial Sequence
  Amino acid sequence derived from Murine monoclonal antibody and further modified by amino acid substitutions
DIWWDDKKSYNPSLKS

SEQ ID NO: 110 - protein, 10 aa, Artificial Sequence
  Amino acid sequence derived from Murine monoclonal antibody and further modified by amino acid substitutions
KLSLRVGYMH

SEQ ID NO: 111 - protein, 16 aa, Artificial Sequence
  Amino acid sequence derived from Murine monoclonal antibody and further modified by amino acid substitutions
DIWWDDKKSYNPSLKD

SEQ ID NO: 112 - protein, 10 aa, Artificial Sequence
  Amino acid sequence derived from Murine monoclonal antibody and further modified by amino acid substitutions
KCSLSVGYMH

SEQ ID NO: 113 - protein, 7 aa, Artificial Sequence
  Amino acid sequence derived from Murine monoclonal antibody and further modified by amino acid substitutions
DTRKLAS

SEQ ID NO: 114 - protein, 16 aa, Artificial Sequence
  Amino acid sequence derived from Murine monoclonal antibody and further modified by amino acid substitutions
DIWWDGKKSYNPSLKS

SEQ ID NO: 115 - protein, 10 aa, Artificial Sequence
  Amino acid sequence derived from Murine monoclonal antibody and further modified by amino acid substitutions
KCSSSVGYMH

SEQ ID NO: 116 - protein, 7 aa, Artificial Sequence
  Amino acid sequence derived from Murine monoclonal antibody and further modified by amino acid substitutions
DTRGLAS

SEQ ID NO: 117 - protein, 10 aa, Artificial Sequence
  Amino acid sequence derived from Murine monoclonal antibody and further modified by amino acid substitutions
KCSSRVGYMH

SEQ ID NO: 118 - protein, 7 aa, Artificial Sequence
  Amino acid sequence derived from Murine monoclonal antibody and further modified by amino acid substitutions
DTRKLPS

SEQ ID NO: 119 - protein, 10 aa, Artificial Sequence
  Amino acid sequence derived from Murine monoclonal antibody and further modified by amino acid substitutions
KCSLRVGYMH

SEQ ID NO: 120 - protein, 10 aa, Artificial Sequence
  Amino acid sequence derived from Murine monoclonal antibody and further modified by amino acid substitutions
SLSLSVGYMH

SEQ ID NO: 121 - protein, 7 aa, Artificial Sequence
  Amino acid sequence derived from Murine monoclonal antibody and further modified by amino acid substitutions
DTMKLAS

SEQ ID NO: 122 - protein, 10 aa, Artificial Sequence
  Amino acid sequence derived from Murine monoclonal antibody and further modified by amino acid substitutions
SLSSSVGYMH

SEQ ID NO: 123 - protein, 7 aa, Artificial Sequence
  Amino acid sequence derived from Murine monoclonal antibody and further modified by amino acid substitutions
DTSRLAS

SEQ ID NO: 124 - protein, 7 aa, Artificial Sequence
  Amino acid sequence derived from Murine monoclonal antibody and further modified by amino acid substitutions
DTSLLAS

SEQ ID NO: 125 - protein, 10 aa, Artificial Sequence
  Amino acid sequence derived from Murine monoclonal antibody and further modified by amino acid substitutions
SLSLRVGYMH

SEQ ID NO: 126 - protein, 7 aa, Artificial Sequence
  Amino acid sequence derived from Murine monoclonal antibody and further modified by amino acid substitutions
DTSLLDS

SEQ ID NO: 127 - protein, 10 aa, Artificial Sequence
  Amino acid sequence derived from Murine monoclonal antibody and further modified by amino acid substitutions
SCQLSVGYMH

SEQ ID NO: 128 - protein, 7 aa, Artificial Sequence
  Amino acid sequence derived from Murine monoclonal antibody and further modified by amino acid substitutions
DTSKLDS

SEQ ID NO: 129 - protein, 10 aa, Artificial Sequence
  Amino acid sequence derived from Murine monoclonal antibody and further modified by amino acid substitutions
SCQSSVGYMH

SEQ ID NO: 130 - protein, 10 aa, Artificial Sequence
  Amino acid sequence derived from Murine monoclonal antibody and further modified by amino acid substitutions
SCQSRVGYMH

SEQ ID NO: 131 - protein, 7 aa, Artificial Sequence
  Amino acid sequence derived from Murine monoclonal antibody and further modified by amino acid substitutions
DTLKLDS

SEQ ID NO: 132 - protein, 10 aa, Artificial Sequence
  Amino acid sequence derived from Murine monoclonal antibody and further modified by amino acid substitutions
SCQLRVGYMH

SEQ ID NO: 133 - protein, 7 aa, Artificial Sequence
  Amino acid sequence derived from Murine monoclonal antibody and further modified by amino acid substitutions
DTLLLAS

SEQ ID NO: 134 - protein, 10 aa, Artificial Sequence
  Amino acid sequence derived from Murine monoclonal antibody and further modified by amino acid substitutions
SLQLSVGYMH

SEQ ID NO: 135 - protein, 7 aa, Artificial Sequence
  Amino acid sequence derived from Murine monoclonal antibody and further modified by amino acid substitutions
DTLKLAS

SEQ ID NO: 136 - protein, 10 aa, Artificial Sequence
  Amino acid sequence derived from Murine monoclonal antibody and further modified by amino acid substitutions
SLQSSVGYMH

SEQ ID NO: 137 - protein, 7 aa, Artificial Sequence
  Amino acid sequence derived from Murine monoclonal antibody and further modified by amino acid substitutions
DTSKLSS

SEQ ID NO: 138 - protein, 10 aa, Artificial Sequence
  Amino acid sequence derived from Murine monoclonal antibody and further modified by amino acid substitutions
SLQSRVGYMH

SEQ ID NO: 139 - protein, 7 aa, Artificial Sequence
  Amino acid sequence derived from Murine monoclonal antibody and further modified by amino acid substitutions
DTSKQAS

SEQ ID NO: 140 - protein, 10 aa, Artificial Sequence
  Amino acid sequence derived from Murine monoclonal antibody and further modified by amino acid substitutions
SLQLRVGYMH

SEQ ID NO: 141 - protein, 7 aa, Artificial Sequence
  Amino acid sequence derived from Murine monoclonal antibody and further modified by amino acid substitutions
DTSKQSS

SEQ ID NO: 142 - protein, 10 aa, Artificial Sequence
  Amino acid sequence derived from Murine monoclonal antibody and further modified by amino acid substitutions
SCSLSVGYMH

SEQ ID NO: 143 - protein, 7 aa, Artificial Sequence
  Amino acid sequence derived from Murine monoclonal antibody and further modified by amino acid substitutions
DTSYLAS

SEQ ID NO: 144 - protein, 10 aa, Artificial Sequence
  Amino acid sequence derived from Murine monoclonal antibody and further modified by amino acid substitutions
SCSSSVGYMH

SEQ ID NO: 145 - protein, 7 aa, Artificial Sequence
  Amino acid sequence derived from Murine monoclonal antibody and further modified by amino acid substitutions
DTSYLSS

SEQ ID NO: 146 - protein, 10 aa, Artificial Sequence
  Amino acid sequence derived from Murine monoclonal antibody and further modified by amino acid substitutions
SCSSRVGYMH

SEQ ID NO: 147 - protein, 7 aa, Artificial Sequence
  Amino acid sequence derived from Murine monoclonal antibody and further modified by amino acid substitutions
DTSYQAS

SEQ ID NO: 148 - protein, 10 aa, Artificial Sequence
  Amino acid sequence derived from Murine monoclonal antibody and further modified by amino acid substitutions
SCSLRVGYMH

SEQ ID NO: 149 - protein, 7 aa, Artificial Sequence
  Amino acid sequence derived from Murine monoclonal antibody and further modified by amino acid substitutions
DTSYQSS

SEQ ID NO: 150 - protein, 10 aa, Artificial Sequence
  Amino acid sequence derived from Murine monoclonal antibody and further modified by amino acid substitutions
KPSSRVGYMH

SEQ ID NO: 151 - protein, 7 aa, Artificial Sequence
  Amino acid sequence derived from Murine monoclonal antibody and further modified by amino acid substitutions
DTMYQAS

SEQ ID NO: 152 - protein, 10 aa, Artificial Sequence
  Amino acid sequence derived from Murine monoclonal antibody and further modified by amino acid substitutions
KPSLRVGYMH

SEQ ID NO: 153 - protein, 10 aa, Artificial Sequence
  Amino acid sequence derived from Murine monoclonal antibody and further modified by amino acid substitutions
KPSSSVGYMH

SEQ ID NO: 154 - protein, 7 aa, Artificial Sequence
  Amino acid sequence derived from Murine monoclonal antibody and further modified by amino acid substitutions
DTMKQAS

SEQ ID NO: 155 - protein, 10 aa, Artificial Sequence
  Amino acid sequence derived from Murine monoclonal antibody and further modified by amino acid substitutions
KPSLSVGYMH

SEQ ID NO: 156 - protein, 7 aa, Artificial Sequence
  Amino acid sequence derived from Murine monoclonal antibody and further modified by amino acid substitutions
DTMKQSS

SEQ ID NO: 157 - protein, 10 aa, Artificial Sequence
  Amino acid sequence derived from Murine monoclonal antibody and further modified by amino acid substitutions
KPQSRVGYMH

SEQ ID NO: 158 - protein, 7 aa, Artificial Sequence
  Amino acid sequence derived from Murine monoclonal antibody and further modified by amino acid substitutions
DTMYLAS

SEQ ID NO: 159 - protein, 10 aa, Artificial Sequence
  Amino acid sequence derived from Murine monoclonal antibody and further modified by amino acid substitutions
KPQLRVGYMH

SEQ ID NO: 160 - protein, 7 aa, Artificial Sequence
  Amino acid sequence derived from Murine monoclonal antibody and further modified by amino acid substitutions
DTMYLSS

SEQ ID NO: 161 - protein, 10 aa, Artificial Sequence
  Amino acid sequence derived from Murine monoclonal antibody and further modified by amino acid substitutions
KPQSSVGYMH

SEQ ID NO: 162 - protein, 7 aa, Artificial Sequence
  Amino acid sequence derived from Murine monoclonal antibody and further modified by amino acid substitutions
DTMKLAS

SEQ ID NO: 163 - protein, 10 aa, Artificial Sequence
  Amino acid sequence derived from Murine monoclonal antibody and further modified by amino acid substitutions
KPQLSVGYMH

SEQ ID NO: 164 - protein, 7 aa, Artificial Sequence
  Amino acid sequence derived from Murine monoclonal antibody and further modified by amino acid substitutions
DTMKLSS

SEQ ID NO: 165 - protein, 7 aa, Artificial Sequence
  Amino acid sequence derived from Murine monoclonal antibody and further modified by amino acid substitutions
DTSKLSS

SEQ ID NO: 166 - protein, 10 aa, Artificial Sequence
  Amino acid sequence derived from Murine monoclonal antibody and further modified by amino acid substitutions
SPSLRVGYMH

SEQ ID NO: 167 - protein, 7 aa, Artificial Sequence
  Amino acid sequence derived from Murine monoclonal antibody and further modified by amino acid substitutions
DTRYQAS

SEQ ID NO: 168 - protein, 10 aa, Artificial Sequence
  Amino acid sequence derived from Murine monoclonal antibody and further modified by amino acid substitutions
SPSSSVGYMH

SEQ ID NO: 169 - protein, 10 aa, Artificial Sequence
  Amino acid sequence derived from Murine monoclonal antibody and further modified by amino acid substitutions
SPSLSVGYMH

SEQ ID NO: 170 - protein, 7 aa, Artificial Sequence
  Amino acid sequence derived from Murine monoclonal antibody and further modified by amino acid substitutions
DTRYQAS

SEQ ID NO: 171 - protein, 10 aa, Artificial Sequence
  Amino acid sequence derived from Murine monoclonal antibody and further modified by amino acid substitutions
SPQSRVGYMH

SEQ ID NO: 172 - protein, 7 aa, Artificial Sequence
  Amino acid sequence derived from Murine monoclonal antibody and further modified by amino acid substitutions
DTRKQSS

SEQ ID NO: 173 - protein, 10 aa, Artificial Sequence
  Amino acid sequence derived from Murine monoclonal antibody and further modified by amino acid substitutions
SPQLRVGYMH

SEQ ID NO: 174 - protein, 7 aa, Artificial Sequence
  Amino acid sequence derived from Murine monoclonal antibody and further modified by amino acid substitutions
DTRKLAS

SEQ ID NO: 175 - protein, 7 aa, Artificial Sequence
  Amino acid sequence derived from Murine monoclonal antibody and further modified by amino acid substitutions
DTRKLSS

SEQ ID NO: 176 - protein, 10 aa, Artificial Sequence
  Amino acid sequence derived from Murine monoclonal antibody and further modified by amino acid substitutions
SPQSSVGYMH

SEQ ID NO: 177 - protein, 10 aa, Artificial Sequence
  Amino acid sequence derived from Murine monoclonal antibody and further modified by amino acid substitutions
SPQLSVGYMH

SEQ ID NO: 178 - protein, 7 aa, Artificial Sequence
  Amino acid sequence derived from Murine monoclonal antibody and further modified by amino acid substitutions
DTRYLAS

SEQ ID NO: 179 - protein, 10 aa, Artificial Sequence
  Amino acid sequence derived from Murine monoclonal antibody and further modified by amino acid substitutions
KAQSRVGYMH

SEQ ID NO: 180 - protein, 10 aa, Artificial Sequence
  Amino acid sequence derived from Murine monoclonal antibody and further modified by amino acid substitutions
KAQLRVGYMH

SEQ ID NO: 181 - protein, 10 aa, Artificial Sequence
  Amino acid sequence derived from Murine monoclonal antibody and further modified by amino acid substitutions
KAQSSVGYMH

SEQ ID NO: 182 - protein, 10 aa, Artificial Sequence
  Amino acid sequence derived from Murine monoclonal antibody and further modified by amino acid substitutions
KAQLSVGYMH

SEQ ID NO: 183 - protein, 10 aa, Artificial Sequence
  Amino acid sequence derived from Murine monoclonal antibody and further modified by amino acid substitutions
KASSRVGYMH

SEQ ID NO: 184 - protein, 10 aa, Artificial Sequence
  Amino acid sequence derived from Murine monoclonal antibody and further modified by amino acid substitutions
KASLRVGYMH

SEQ ID NO: 185 - protein, 10 aa, Artificial Sequence
  Amino acid sequence derived from Murine monoclonal antibody and further modified by amino acid substitutions
KASSSVGYMH

SEQ ID NO: 186 - protein, 10 aa, Artificial Sequence
  Amino acid sequence derived from Murine monoclonal antibody and further modified by amino acid substitutions
KASLSVGYMH

SEQ ID NO: 187 - protein, 10 aa, Artificial Sequence
  Amino acid sequence derived from Murine monoclonal antibody and further modified by amino acid substitutions
SASLRVGYMH

SEQ ID NO: 188 - protein, 10 aa, Artificial Sequence
  Amino acid sequence derived from Murine monoclonal antibody and further modified by amino acid substitutions
SASLSVGYMH

SEQ ID NO: 189 - protein, 10 aa, Artificial Sequence
  Amino acid sequence derived from Murine monoclonal antibody and further modified by amino acid substitutions
SAQSRVGYMH

SEQ ID NO: 190 - protein, 10 aa, Artificial Sequence
  Amino acid sequence derived from Murine monoclonal antibody and further modified by amino acid substitutions
SAQLRVGYMH

SEQ ID NO: 191 - protein, 10 aa, Artificial Sequence
  Amino acid sequence derived from Murine monoclonal antibody and further modified by amino acid substitutions
SAQSSVGYMH

SEQ ID NO: 192 - protein, 10 aa, Artificial Sequence
  Amino acid sequence derived from Murine monoclonal antibody and further modified by amino acid substitutions
LPSLSVGYMH

SEQ ID NO: 193 - protein, 10 aa, Artificial Sequence
  Amino acid sequence derived from Murine monoclonal antibody and further modified by amino acid substitutions
LPSSSVGYMH

SEQ ID NO: 194 - protein, 10 aa, Artificial Sequence
  Amino acid sequence derived from Murine monoclonal antibody and further modified by amino acid substitutions
LPSLRVGYMH

SEQ ID NO: 195 - protein, 10 aa, Artificial Sequence
  Amino acid sequence derived from Murine monoclonal antibody and further modified by amino acid substitutions
LCSSRVGYMH

SEQ ID NO: 196 - protein, 10 aa, Artificial Sequence
  Amino acid sequence derived from Murine monoclonal antibody and further modified by amino acid substitutions
LCSLSVGYMH

SEQ ID NO: 197 - protein, 10 aa, Artificial Sequence
  Amino acid sequence derived from Murine monoclonal antibody and further modified by amino acid substitutions
LCSSSVGYMH

SEQ ID NO: 198 - protein, 10 aa, Artificial Sequence
  Amino acid sequence derived from Murine monoclonal antibody and further modified by amino acid substitutions
LCSLRVGYMH

SEQ ID NO: 199 - protein, 10 aa, Artificial Sequence
  Amino acid sequence derived from Murine monoclonal antibody and further modified by amino acid substitutions
LPQSRVGYMH

SEQ ID NO: 200 - protein, 10 aa, Artificial Sequence
  Amino acid sequence derived from Murine monoclonal antibody and further modified by amino acid substitutions
LPQLSVGYMH

SEQ ID NO: 201 - protein, 10 aa, Artificial Sequence
  Amino acid sequence derived from Murine monoclonal antibody and further modified by amino acid substitutions
LPQSSVGYMH

SEQ ID NO: 202 - protein, 10 aa, Artificial Sequence
  Amino acid sequence derived from Murine monoclonal antibody and further modified by amino acid substitutions
LPQLRVGYMH

SEQ ID NO: 203 - protein, 10 aa, Artificial Sequence
  Amino acid sequence derived from Murine monoclonal antibody and further modified by amino acid substitutions
LCQSRVGYMH

SEQ ID NO: 204 - protein, 10 aa, Artificial Sequence
  Amino acid sequence derived from Murine monoclonal antibody and further modified by amino acid substitutions
LCQLSVGYMH

SEQ ID NO: 205 - protein, 10 aa, Artificial Sequence
  Amino acid sequence derived from Murine monoclonal antibody and further modified by amino acid substitutions
LCQSSVGYMH

SEQ ID NO: 206 - protein, 10 aa, Artificial Sequence
  Amino acid sequence derived from Murine monoclonal antibody and further modified by amino acid substitutions
LCQLRVGYMH

SEQ ID NO: 207 - protein, 10 aa, Artificial Sequence
  Amino acid sequence derived from Murine monoclonal antibody and further modified by amino acid substitutions
SAQLSVGYMH

SEQ ID NO: 208 - protein, 450 aa, Artificial Sequence
  Humanized antibody - VH Chain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 213 aa, Artificial Sequence
  Humanized antibody - VL Chain
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARSMITNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 213 aa, Artificial Sequence
  Humanized antibody - VL Chain
DIQMTQSPSTLSASVGDRVTITCSASSSVGYMHWYQQKPGKAPKLLIYDTFKLASGVPSRFSGSGSGTEFTLTISSLQPDDFATYYCFQFSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 450 aa, Artificial Sequence
  Humanized antibody - VH Chain
QVTLRESGPALVKPTQTLTLTCTFSGFSLSTPGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 213 aa, Artificial Sequence
  Humanized antibody - VL Chain
DIQMTQSPSTLSASVGDRVTITCSLSSRVGYMHWYQQKPGKAPKLLIYDTFYLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 450 aa, Artificial Sequence
  Humanized antibody - VH Chain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 213 aa, Artificial Sequence
  Humanized antibody - VL Chain
DIQMTQSPSTLSASVGDRVTITCSLSSRVGYMHWYQQKPGKAPKLLIYDTRGLPSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 450 aa, Artificial Sequence
  Humanized antibody - VH Chain
QVTLRESGPALVKPTQTLTLTCTFSGFSLSTPGMSVGWIRQPPGKALEWLADIWWDGKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 213 aa, Artificial Sequence
  Humanized antibody - VL Chain
DIQMTQSPSTLSASVGDRVTITCSPSSRVGYMHWYQQKPGKAPKLLIYDTMRLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 450 aa, Artificial Sequence
  Humanized antibody - VH Chain
QVTLRESGPALVKPTQTLTLTCTFSGFSLSTAGMSVGWIRQPPGKALEWLADIWWDGKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 213 aa, Artificial Sequence
  Humanized antibody - VL Chain
DIQMTQSPSTLSASVGDRVTITCSLSSRVGYMHWYQQKPGKAPKLLIYDTFKLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 450 aa, Artificial Sequence
  Humanized antibody - VH Chain
QVTLRESGPALVKPTQTLTLTCTFSGFSLSTAGMSVGWIRQPPGKALEWLADIWWDGKKDYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 213 aa, Artificial Sequence
  Humanized antibody - VL Chain
DIQMTQSPSTLSASVGDRVTITCSASSRVGYMHWYQQKPGKAPKLLIYDTFKLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 450 aa, Artificial Sequence
  Humanized antibody - VH Chain
QVTLRESGPALVKPTQTLTLTCTFSGFSLSTAGMSVGWIRQPPGKALEWLADIWWDGKKSYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 213 aa, Artificial Sequence
  Humanized antibody - VL Chain
DIQMTQSPSTLSASVGDRVTITCSLSSRVGYMHWYQQKPGKAPKLLIYDTMYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 213 aa, Artificial Sequence
  Humanized antibody - VL Chain
DIQMTQSPSTLSASVGDRVTITCLPSSRVGYMHWYQQKPGKAPKLLIYDTMYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 213 aa, Artificial Sequence
  Humanized antibody - VL Chain
DIQMTQSPSTLSASVGDRVTITCSASSRVGYMHWYQQKPGKAPKLLIYDTFFLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 213 aa, Artificial Sequence
  Humanized antibody - VL Chain
DIQMTQSPSTLSASVGDRVTITCSPSSRVGYMHWYQQKPGKAPKLLIYDTRYQSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 450 aa, Artificial Sequence
  Humanized antibody - VH Chain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 213 aa, Artificial Sequence
  Humanized antibody - VL Chain
DIQMTQSPSTLSASVGDRVTITCSASSSVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 213 aa, Artificial Sequence
  Humanized antibody - VL Chain
DIQMTQSPSTLSASVGDRVTITCSASSSVGYMHWYQQKPGKAPKLLIYDTFKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 450 aa, Artificial Sequence; identical to SEQ ID NO: 232
  Humanized antibody - VH Chain

SEQ ID NO: 235 - protein, 213 aa, Artificial Sequence
  Humanized antibody - VL Chain
DIQMTQSPSTLSASVGDRVTITCSASSSVGYMHWYQQKPGKAPKLLIYDTYKQTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 450 aa, Artificial Sequence; identical to SEQ ID NO: 232
  Humanized antibody - VH Chain

SEQ ID NO: 237 - protein, 213 aa, Artificial Sequence
  Humanized antibody - VL Chain
DIQMTQSPSTLSASVGDRVTITCSASSSVGYMHWYQQKPGKAPKLLIYDTRYLSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKDYNPSLKSRLTISKDTSKNQVVLKVTNMDPADTATYYCARDMITNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 213 aa, Artificial Sequence; identical to SEQ ID NO: 233
  Humanized antibody - VL Chain

SEQ ID NO: 240 - protein, 450 aa, Artificial Sequence; identical to SEQ ID NO: 210
  Humanized antibody - VH Chain

SEQ ID NO: 241 - protein, 213 aa, Artificial Sequence
  Humanized antibody - VH Chain
DIQMTQSPSTLSASVGDRVTITCSASSSVGYMHWYQQKPGKAPKLLIYDTFKLASGVPSRFSGSGSGTEFTLTISSLQPDDFATYYCFQGSFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 450 aa, Artificial Sequence; identical to SEQ ID NO: 238
  Humanized antibody - VH Chain

SEQ ID NO: 243 - protein, 213 aa, Artificial Sequence
  Humanized antibody - VL Chain
DIQMTQSPSTLSASVGDRVTITCSASSSVGYMHWYQQKPGKAPKLLIYDTFKLTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 450 aa, Artificial Sequence; identical to SEQ ID NO: 238
  Humanized antibody - VH Chain

SEQ ID NO: 245 - protein, 213 aa, Artificial Sequence
  Humanized antibody - VL Chain
DIQMTQSPSTLSASVGDRVTITCSASSRVGYMHWYQQKPGKAPKLLIYDTFKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 450 aa, Artificial Sequence; identical to SEQ ID NO: 238
  Humanized antibody - VH Chain

SEQ ID NO: 247 - protein, 213 aa, Artificial Sequence
  Humanized antibody - VL Chain
DIQMTQSPSTLSASVGDRVTITCSASSSVGYMHWYQQKPGKAPKLLIYDTFRLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 213 aa, Artificial Sequence
  Humanized antibody - VL Chain
DIQMTQSPSTLSASVGDRVTITCSPSSRVGYMHWYQQKPGKAPKLLIYDTYRHSSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 450 aa, Artificial Sequence; identical to SEQ ID NO: 218
  Humanized antibody - VH Chain

SEQ ID NO: 251 - protein, 213 aa, Artificial Sequence
  Humanized antibody - VL Chain
DIQMTQSPSTLSASVGDRVTITCSLSSSVGYMHWYQQKPGKAPKLLIYDTFFHRSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 450 aa, Artificial Sequence; identical to SEQ ID NO: 226
  Humanized antibody - VH Chain

SEQ ID NO: 253 - protein, 213 aa, Artificial Sequence
  Humanized antibody - VL Chain
DIQMTQSPSTLSASVGDRVTITCSASSRVGYMHWYQQKPGKAPKLLIYDTLLLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 450 aa, Artificial Sequence; identical to SEQ ID NO: 226
  Humanized antibody - VH Chain

SEQ ID NO: 255 - protein, 213 aa, Artificial Sequence
  Humanized antibody - VL Chain
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 450 aa, Artificial Sequence; identical to SEQ ID NO: 226
  Humanized antibody - VH Chain

SEQ ID NO: 257 - protein, 213 aa, Artificial Sequence
  Humanized antibody - VL Chain
DIQMTQSPSTLSASVGDRVTITCSASSRVGYMHWYQQKPGKAPKLLIYDTSFLDSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - DNA, 26 bp, Artificial
  Description of Artificial Sequence Primer
agtgtcttaaccagcaaagtgttaga

SEQ ID NO: 259 - DNA, 26 bp, Artificial Sequence
  Description of Artificial Sequence Primer
tcattgacttgagatattgatgcatc

SEQ ID NO: 260 - protein, 15 aa, Artificial Sequence
  Linker for constructing humanized antibodies
GGGGSGGGGSGGGGS

SEQ ID NO: 261 - protein, 15 aa, Artificial Sequence
  Linker for constructing humanized antibodies
ESGRSGGGGSGGGGS

SEQ ID NO: 262 - protein, 14 aa, Artificial Sequence
  Linker for constructing humanized antibodies
EGKSSGSGSESKST

SEQ ID NO: 263 - protein, 15 aa, Artificial Sequence
  Linker for constructing humanized antibodies
EGKSSGSGSESKSTQ

SEQ ID NO: 264 - protein, 14 aa, Artificial Sequence
  Linker for constructing humanized antibodies
EGKSSGSGSESKVD

SEQ ID NO: 265 - protein, 14 aa, Artificial Sequence
  Linker for constructing humanized antibodies
GSTSGSGKSSEGKG

SEQ ID NO: 266 - protein, 18 aa, Artificial Sequence
  Linker for constructing humanized antibodies
KESGSVSSEQLAQFRSLD

SEQ ID NO: 267 - protein, 16 aa, Artificial Sequence
  Linker for constructing humanized antibodies
ESGSVSSEELAFRSLD

SEQ ID NO: 268 - protein, 4 aa, Homo sapiens
  intrabody
KDEL

SEQ ID NO: 269 - protein, 4 aa, Homo sapiens
  intrabody
DDEL

SEQ ID NO: 270 - protein, 4 aa, Homo sapiens
  intrabody
DEEL

SEQ ID NO: 271 - protein, 4 aa, Homo sapiens
  intrabody
QEDL

SEQ ID NO: 272 - protein, 4 aa, Homo sapiens
  intrabody
RDEL

SEQ ID NO: 273 - protein, 7 aa, Homo sapiens
  intrabody
PKKKRKV

SEQ ID NO: 274 - protein, 7 aa, Homo sapiens
  intrabody
PQKKIKS

SEQ ID NO: 275 - protein, 5 aa, Homo sapiens
  intrabody
QPKKP

SEQ ID NO: 276 - protein, 4 aa, Homo sapiens
  intrabody
RKKR

SEQ ID NO: 277 - protein, 5 aa, Homo sapiens
  intrabody
KKKRK

SEQ ID NO: 278 - protein, 12 aa, Homo sapiens
  intrabody
RKKRRQRRRAHQ

SEQ ID NO: 279 - protein, 16 aa, Homo sapiens
  intrabody
RQARRNRRRRWRERQR

SEQ ID NO: 280 - protein, 19 aa, Homo sapiens
  intrabody
MPLTRRRPAASQALAPPTP

SEQ ID NO: 281 - protein, 15 aa, Homo sapiens
  intrabody
MDDQRDLISNNEQLP

SEQ ID NO: 282 - protein, 32 aa, Homo sapiens
  intrabody
MLFNLRXXLNNAAFRHGHNFMVRNFRCGQPLX

SEQ ID NO: 283 - protein, 3 aa, Homo sapiens
  intrabody
AKL

SEQ ID NO: 284 - protein, 6 aa, Homo sapiens
  intrabody
SDYQRL

SEQ ID NO: 285 - protein, 8 aa, Homo sapiens
  intrabody
GCVCSSNP

SEQ ID NO: 286 - protein, 8 aa, Homo sapiens
  intrabody
GQTVTTPL

SEQ ID NO: 287 - protein, 8 aa, Homo sapiens
  intrabody
GQELSQHE

SEQ ID NO: 288 - protein, 8 aa, Homo sapiens
  intrabody
GNSPSYNP

SEQ ID NO: 289 - protein, 8 aa, Homo sapiens
  intrabody
GVSGSKGQ

SEQ ID NO: 290 - protein, 8 aa, Homo sapiens
  intrabody
GQTITTPL

SEQ ID NO: 291 - protein, 8 aa, Homo sapiens
  intrabody
GQTLTTPL

SEQ ID NO: 292 - protein, 8 aa, Homo sapiens
  intrabody
GQIFSRSA

SEQ ID NO: 293 - protein, 8 aa, Homo sapiens
  intrabody
GQIHGLSP

SEQ ID NO: 294 - protein, 8 aa, Homo sapiens
  intrabody
GARASVLS

SEQ ID NO: 295 - protein, 8 aa, Homo sapiens
  intrabody
GCTLSAEE

SEQ ID NO: 296 - protein, 16 aa, Homo sapiens
  intrabody
AAVALLPAVLLALLAP

SEQ ID NO: 297 - protein, 12 aa, Homo sapiens
  intrabody
AAVLLPVLLAAP

SEQ ID NO: 298 - protein, 15 aa, Homo sapiens
  intrabody
VTVLALGALAGVGVG

SEQ ID NO: 299 - DNA, 30 bp, Artificial Sequence
  Description of Artificial Sequence Primer
ccagcagtaccacttccttgccctgcgccg

SEQ ID NO: 300 - DNA, 30 bp, Artificial Sequence
  Description of Artificial Sequence Primer
gccgcgtcccgttccttcaccatgacgacc

SEQ ID NO: 301 - DNA, 31 bp, Artificial Sequence
  Description of Artificial Sequence Primer
ccagcagtaccgcttccttgccctgcggccg

SEQ ID NO: 302 - DNA, 30 bp, Artificial Sequence; identical to SEQ ID NO: 300
  Description of Artificial Sequence Primer

SEQ ID NO: 303 - protein, 450 aa, Artificial Sequence
  Humanized antibody - VH Chain
QVTLRESGPALVKPTQTLTLTCTFSGFSLSTAGMSVGWIRQPPGKALEWLADIWWGDKGHYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 120 aa, Artificial Sequence
  Humanized antibody - VH Domain
QVTLRESGPALVKPTQTLTLTCTFSGFSLSTAGMSVGWIRQPPGKALEWLADIWWGDKGHYNPSLKDRLTISKDTSKNQVVLKVTNMDPADTATYYCARDMIFNWYFDVWGQGTTVTVSS

SEQ ID NO: 305 - protein, 16 aa, Artificial Sequence
  Amino acid sequence derived from Murine monoclonal antibody and further modified by amino acid substitutions
DIWWGDKGHYNPSLKD

SEQ ID NO: 306 - protein, 213 aa, Artificial Sequence
  Humanized antibody - VL Chain
DIQMTQSPSTLSASVGDRVTITCSASSSVGYMHWYQQKPGKAPKLLIYDTFYLHSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 106 aa, Artificial Sequence
  Humanized antibody - VL Domain
DIQMTQSPSTLSASVGDRVTITCSASSSVGYMHWYQQKPGKAPKLLIYDTFYLHSGVPSRFSGSGSGTEFTLTISSLQPDDFATYYCFQGSGYPFTFGGGTKVEIK

SEQ ID NO: 308 - protein, 7 aa, Artificial Sequence
  Amino acid sequence derived from Murine monoclonal antibody and further modified by amino acid substitutions
DTFYLHS

SEQ ID NO: 309 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMITNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKSYNPSLKDRLTISKDTSKNQVVLKVTNMDPADTATYYCARDMITNWYFDVWGQGTTVTVSS

SEQ ID NO: 311 - protein, 10 aa, Artificial Sequence
  Amino acid sequence derived from Murine monoclonal antibody and further modified by amino acid substitutions
DMITNWYFDV

SEQ ID NO: 312 - protein, 213 aa, Artificial Sequence
  Humanized antibody - VL Chain
DIQMTQSPSTLSASVGDRVTITCLLSSRVGYMHWYQQKPGKAPKLLIYDTYYQTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 106 aa, Artificial Sequence
  Humanized antibody - VL Domain
DIQMTQSPSTLSASVGDRVTITCLLSSRVGYMHWYQQKPGKAPKLLIYDTYYQTSGVPSRFSGSGSGTEFTLTISSLQPDDFATYYCFQGSGYPFTFGGGTKVEIK

SEQ ID NO: 314 - protein, 10 aa, Artificial Sequence
  Amino acid sequence derived from Murine monoclonal antibody and further modified by amino acid substitutions
LLSSRVGYMH

SEQ ID NO: 315 - protein, 7 aa, Artificial Sequence
  Amino acid sequence derived from Murine monoclonal antibody and further modified by amino acid substitutions
DTYYQTS

SEQ ID NO: 316 - protein, 450 aa, Artificial Sequence; identical to SEQ ID NO: 248
  Humanized antibody - VH Chain

SEQ ID NO: 317 - protein, 120 aa, Artificial Sequence; identical to SEQ ID NO: 67
  Humanized antibody - VH Domain

SEQ ID NO: 318 - protein, 213 aa, Artificial Sequence
  Humanized antibody - VL Chain
DIQMTQSPSTLSASVGDRVTITCLLSSRVGYMHWYQQKPGKAPKLLIYDTMYQASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 106 aa, Artificial Sequence
  Humanized antibody - VL Domain
DIQMTQSPSTLSASVGDRVTITCLLSSRVGYMHWYQQKPGKAPKLLIYDTMYQASGVPSRFSGSGSGTEFTLTISSLQPDDFATYYCFQGSGYPFTFGGGTKVEIK

SEQ ID NO: 320 - protein, 10 aa, Artificial Sequence
  Amino acid sequence derived from Murine monoclonal antibody and further modified by amino acid substitutions
LLSSRVGYMH

SEQ ID NO: 321 - protein, 7 aa, Artificial Sequence
  Amino acid sequence derived from Murine monoclonal antibody and further modified by amino acid substitutions
DTMYQAS

SEQ ID NO: 322 - protein, 450 aa, Artificial Sequence; identical to SEQ ID NO: 248
  Humanized antibody - VH Chain

SEQ ID NO: 323 - protein, 120 aa, Artificial Sequence; identical to SEQ ID NO: 67
  Humanized antibody - VH Domain

SEQ ID NO: 324 - protein, 213 aa, Artificial Sequence
  Humanized antibody - VL Chain
DIQMTQSPSTLSASVGDRVTITCSLSSRVGYMHWYQQKPGKAPKLLIYDTYYLPSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 106 aa, Artificial Sequence
  Humanized antibody - VL Domain
DIQMTQSPSTLSASVGDRVTITCSLSSRVGYMHWYQQKPGKAPKLLIYDTYYLPSGVPSRFSGSGSGTEFTLTISSLQPDDFATYYCFQGSGYPFTFGGGTKVEIK

SEQ ID NO: 326 - protein, 7 aa, Artificial Sequence
  Amino acid sequence derived from Murine monoclonal antibody and further modified by amino acid substitutions
DTYYLPS

SEQ ID NO: 327 - protein, 450 aa, Artificial Sequence
  Humanized antibody - VH Chain
QVTLRESGPALVKPTQTLTLTCTFSGFSLSTAGMSVGWIRQPPGKALEWLADIWWDDKKDYNPSLKDRLTISKDTSKNQVVLKVTNMDPADTATYYCARDMIFNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 120 aa, Artificial Sequence
  Humanized antibody - VH Domain
QVTLRESGPALVKPTQTLTLTCTFSGFSLSTAGMSVGWIRQPPGKALEWLADIWWDDKKDYNPSLKDRLTISKDTSKNQVVLKVTNMDPADTATYYCARDMIFNWYFDVWGQGTTVTVSS

SEQ ID NO: 329 - protein, 16 aa, Artificial Sequence
  Amino acid sequence derived from Murine monoclonal antibody and further modified by amino acid substitutions
DIWWDDKKDYNPSLKD

SEQ ID NO: 330 - protein, 213 aa, Artificial Sequence
  Humanized antibody - VL Chain
DIQMTQSPSTLSASVGDRVTITCSLSSRVGYMHWYQQKPGKAPKLLIYDTFRHTSGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 106 aa, Artificial Sequence
  Humanized antibody - VL Domain
DIQMTQSPSTLSASVGDRVTITCSLSSRVGYMHWYQQKPGKAPKLLIYDTFRHTSGVPSRFSGSGSGTEFTLTISSLQPDDFATYYCFQGSGYPFTFGGGTKVEIK

SEQ ID NO: 332 - protein, 7 aa, Artificial Sequence
  Amino acid sequence derived from Murine monoclonal antibody and further modified by amino acid substitutions
DTFRHTS

SEQ ID NO: 333 - protein, 213 aa, Artificial Sequence
  Humanized antibody - VL Chain
DIQMTQSPSTLSASVGDRVTITCSPSSSVGYMHWYQQKPGKAPKLLIYDTYYLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 106 aa, Artificial Sequence
  Humanized antibody - VL Domain
DIQMTQSPSTLSASVGDRVTITCSPSSSVGYMHWYQQKPGKAPKLLIYDTYYLASGVPSRFSGSGSGTEFTLTISSLQPDDFATYYCFQGSGYPFTFGGGTKVEIK

SEQ ID NO: 335 - protein, 10 aa, Artificial Sequence
  Amino acid sequence derived from Murine monoclonal antibody and further modified by amino acid substitutions
SPSSSVGYMH

SEQ ID NO: 336 - protein, 7 aa, Artificial Sequence
  Amino acid sequence derived from Murine monoclonal antibody and further modified by amino acid substitutions
DTYYLAS

SEQ ID NO: 337 - protein, 365 aa, Homo sapiens
  Human FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 338 - protein, 365 aa, Murine
MGMPLPWALSLLLVLLPQTWGSETRPPLMYHLTAVSNPSTGLPSFWATGWLGPQQYLTYNSLRQEADPCGAWVWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 339 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 340 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 15 aa, Homo sapiens
EPKSCDKTHTCPPCP

SEQ ID NO: 342 - protein, 232 aa, Homo sapiens
  Human hinge Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 120 aa, Artificial Sequence
  Humanized antibody - VH Domain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGQGTTVTVSS

(19) United States
(12) Patent Application Publication
(10) Pub. No.: US 2011/0008329 A1
(43) Pub. Date: Jan. 13, 2011

(54) Methods of Treating RSV Infections And Related Conditions

(75) Inventors: Subramaniam Krishnan, Montgomery Village, MD (US); JoAnn Suzich, Washington Grove, MD (US); Peter Kiener, Potomac, MD (US); Genevieve Losonsky, Phoenix, MD (US); Herren Wu, Boyds, MD (US); William Dall'Acqua, Gaithersburg, MD (US); Bettina Richter, Gaithersburg, MD (US)

Correspondence Address: MEDIMMUNE, LLC;Patrick Scott Alban, ONE MEDIMMUNE WAY, GAITHERSBURG, MD, 20878, US

(73) Assignee: Medlmmune, LLC, Gaithersburg, MD (US)

(21) Appl. No.: 12/600,292

(22) PCT Filed: Jun. 25, 2008
(86) PCT No.: PCT/US08/68155

§ 371 Date: Sep. 14, 2010

Related U.S. Application Data
(60) Provisional application No. 60/946,271, filed on Jun. 26, 2007.
(60) Provisional application No. 60/953,260, filed on Aug. 1, 2007.
(60) Provisional application No. 61/054,927, filed on May 21, 2008.

(51) Int. Cl. A61K 39/395; C07K 16/10; A61P 31/14; A61P 37/04

(52) U.S. Cl. 424/133.1; 530/387.3

(57) ABSTRACT

The present invention provides methods for managing, treating and/or ameliorating a respiratory syncytial virus (RSV) infection (e.g., acute RSV disease, or a RSV upper respiratory tract infection (URI) and/or lower respiratory tract infection (LRI)), and/or a symptom or a long-term respiratory condition relating thereto (e.g., asthma, wheezing, reactive airway disease (RAD), or chronic obstructive pulmonary disease (COPD)) in a subject, comprising administering to said human an effective amount of one or more antibodies that immunospecifically bind to one or more RSV antigens with a high affinity and/or high avidity and further comprise a modified IgG constant domain, or FcRn-binding fragment thereof, to not only decrease RSV infection, but also decrease the pro-inflammatory epithelial cell immune responses in order to mitigate the later development of asthma and/or wheezing and/or COPD in said patient.

### 1. INTRODUCTION

The present invention relates to compositions comprising antibodies or fragments thereof that immunospecifically bind to a RSV antigen and methods for treating or ameliorating symptoms and/or long term consequences associated with respiratory syncytial virus (RSV) infection utilizing said compositions. In particular, the present invention relates to methods for treating or ameliorating symptoms and/or long term consequences associated with RSV infection, said methods comprising administering to a human subject an effective amount of one or more antibodies or fragments thereof that immunospecifically bind to a RSV antigen, wherein a certain serum titer of said antibodies or antibody fragments is achieved in said human subject. The present invention provides Fc modified antibodies that immunospecifically bind to a respiratory syncytial virus (RSV) antigen with high affinity and/or high avidity. The invention also provides methods of managing, treating and/or ameliorating a RSV infection (e.g., acute RSV disease, or a RSV upper respiratory tract infection (URI) and/or lower respiratory tract infection (LRI)), said methods comprising administering to a human subject an effective amount of one or more of the Fc modified antibodies (e.g., one or more modified antibodies) provided herein. The present invention further provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof by administering a therapeutically effective amount of the antibodies of the invention. The present invention also relates to detectable or diagnostic compositions comprising antibodies or fragments thereof that immunospecifically bind to a RSV antigen and methods for detecting or diagnosing RSV infection utilizing said compositions.

### 2. BACKGROUND OF THE INVENTION

### Respiratory Syncytial Virus

Respiratory infections are common infections of the upper respiratory tract (e.g., nose, ears, sinuses, and throat) and lower respiratory tract (e.g., trachea, bronchial tubes, and lungs). Symptoms of upper respiratory infection include runny or stuffy nose, irritability, restlessness, poor appetite, decreased activity level, coughing, and fever. Viral upper respiratory infections cause and/or are associated with sore throats, colds, croup, and the flu. Clinical manifestations of a lower respiratory infection include shallow coughing that produces sputum in the lungs, fever, and difficulty breathing.

Respiratory syncytial virus (RSV) is one of the leading causes of respiratory disease worldwide. In the United States, it is responsible for tens of thousands of hospitalizations and thousands of deaths per year (see Black, C. P., Resp. Care 2003 48(3):209-31 for a recent review of the biology and management of RSV). Infants and children are most at risk for serious RSV infections which migrate to the lower respiratory system, resulting in pneumonia or bronchiolitis. In fact, 80% of childhood bronchiolitis cases and 50% of infant pneumonias are attributable to RSV. The virus is so ubiquitous and highly contagious that almost all children have been infected by two years of age. Although infection does not produce lasting immunity, reinfections tend to be less severe so that in older children and healthy adults RSV manifests itself as a cold or flu-like illness affecting the upper and/or lower respiratory system, without progressing to serious lower respiratory tract involvement. However, RSV infections can become serious in elderly or immunocompromised adults. (Evans, A. S., eds., 1989, Viral Infections of Humans. Epidemiology and Control, 3^{rd} ed., Plenum Medical Book, New York at pages 525-544; Falsey, A. R., 1991, Infect. Control Hosp. Epidemiol. 12:602-608; and Garvie et al., 1980, Br. Med. J. 281:1253-1254; Hertz et al., 1989, Medicine 68:269-281).

While a vaccine or commercially available treatment are not yet available, some success has been achieved in the area of prevention for infants at high risk of serious lower respiratory tract disease caused by RSV, as well as a reduction of LRI. In particular, there are two immunoglobulin-based therapies approved to protect high-risk infants from serious LRI: RSV-IGIV (RSV-immunoglobulin intravenous, also known as RespiGam™) and palivizumab (SYNAGIS®). However, neither RSV-IGIV nor palivizumab has been approved for use other than as a prophylactic agent for serious lower respiratory tract acute RSV disease.

RSV is easily spread by physical contact with contaminated secretions. The virus can survive for at least half an hour on hands and for hours on countertops and used tissues. The highly contagious nature of RSV is evident from the risk factors associated with contracting serious infections. One of the greatest risk factors is hospitalization, where in some cases in excess of 50% of the staff on pediatric wards were found to be infected (Black, C. P., Resp. Care 2003 48(3):209-31). Up to 20% of these adult infections are asymptomatic but still produce substantial shedding of the virus. Other risk factors include attendance at day care centers, crowded living conditions, and the presence of school-age siblings in the home.

Because, as discussed above, RSV is not simply an illness confined to high-risk infants, it is useful to explore RSV therapy, as opposed to prophylaxis, as an alternative treatment for low-risk pediatric and high risk adult populations. However treatment options for established RSV disease are limited. Severe RSV disease of the lower respiratory tract often requires considerable supportive care, including administration of humidified oxygen and respiratory assistance (Fields et al., eds, 1990, Fields Virology, 2^{nd} ed., Vol. 1, Raven Press, New York at pages 1045-1072). The only drug approved for treatment of infection is the antiviral agent ribavirin (American Academy of Pediatrics Committee on Infectious Diseases, 1993, Pediatrics 92:501-504). It has been shown to be effective in the treatment of RSV pneumonia and bronchiolitis, modifying the course of severe RSV disease in immunocompetent children (Smith et al., 1991, New Engl. J. Med. 325:24-29). However, ribavirin has had limited use because it requires prolonged aerosol administration and because of concerns about its potential risk to pregnant women who may be exposed to the drug during its administration in hospital settings.

Clinical studies have been conducted exploring treatment of RSV using palivizumab. Malley and his colleagues studied the anti-viral effects of palivizumab on the lower respiratory tract RSV concentrations in RSV-infected, intubated infants with severe RSV disease, before and after a single infusion of 15 mg/kg palivizumab or placebo (see Malley, R. et al., The Journal of Infectious Diseases, 1998; 178:1555-1561). In that study, statistically significant reduction in lung viral titers were observed, but there was no improvement in the duration of RSV hospitalization, the days on supplemental oxygen therapy, or hospital days with high lower respiratory infection scores.

In another study by Saez-Llorens and colleagues, a phase I/II clinical trial was conducted to describe the safety, tolerance, pharmacokinetics and clinical outcome of a single intravenous 15-mg/kg dose of palivizumab in previously healthy children hospitalized with acute RSV infection. While the study concluded that intravenous palivizumab was safe and well-tolerated in children hospitalized with RSV disease, there were no significant differences in clinical outcomes (i.e., no improvement in the duration of RSV hospitalization, the days on supplemental oxygen therapy, or hospital days with high lower respiratory infection scores), between placebo and palivizumab groups.

One way to improve the treatment outcomes and options would be to develop one or more highly potent RSV neutralizing monoclonal antibodies (MAbs). Such MAbs should be human or humanized in order to retain favorable pharmacokinetics and to avoid generating a human anti-mouse antibody response, as repeat dosing would be required throughout the RSV season. One such antibody, motavizumab or MEDI-524, see Wu et al., J. Mol. Biol. 368:652-655 (2007)), results in a more successful clinical outcome in a treatment setting, as opposed to prophylaxis. It is postulated that an effective treatment of RSV in low-risk infants may mitigate the later development of respiratory illnesses or long term consequences, such as asthma, reactive airway disease (RAD), wheezing and/or chronic obstructive pulmonary disease (COPD).

Asthma and Reactive Airway Disease (RAD)

About 12 million people in the U.S. have asthma and it is the leading cause of hospitalization for children. The Merck Manual of Diagnosis and Therapy (17th ed., 1999).

Asthma is an inflammatory disease of the lung that is characterized by airway hyperresponsiveness (“AHR”), bronchoconstriction (i.e., wheezing), eosinophilic inflammation, mucus hypersecretion, subepithelial fibrosis, and elevated IgE levels. Asthmatic attacks can be triggered by environmental triggers (e.g., acarids, insects, animals (e.g., cats, dogs, rabbits, mice, rats, hamsters, guinea pigs, mice, rats, and birds), fungi, air pollutants (e.g., tobacco smoke), irritant gases, fumes, vapors, aerosols, chemicals, or pollen), exercise, or cold air. The cause(s) of asthma is unknown. However, it has been speculated that family history of asthma (London et al., 2001, Epidemiology 12(5):577-83), early exposure to allergens, such as dust mites, tobacco smoke, and cockroaches (Melen et al., 2001, 56(7):646-52), and respiratory infections (Wenzel et al., 2002, Am J Med, 112(8):672-33 and Lin et al., 2001, J Microbiol Immuno Infect, 34(4):259-64), such as RSV, may increase the risk of developing asthma. A review of asthma, including risk factors, animal models, and inflammatory markers can be found in O'Byrne and Postma (1999), Am. J. Crit. Care. Med. 159:S41-S66, which is incorporated herein by reference in its entirety.

Current therapies are mainly aimed at managing asthma and include the administration of β-adrenergic drugs (e.g., epinephrine and isoproterenol), theophylline, anticholinergic drugs (e.g., atropine and ipratorpium bromide), corticosteroids, and leukotriene inhibitors. These therapies are associated with side effects such as drug interactions, dry mouth, blurred vision, growth suppression in children, and osteoporosis in menopausal women. Cromolyn and nedocromil are administered prophylactically to inhibit mediator release from inflammatory cells, reduce airway hyperresponsiveness, and block responses to allergens. However, there are no current therapies available that prevent the development of asthma in subjects at increased risk of developing asthma. Thus, new therapies with fewer side effects and better therapeutic efficacy are needed for asthma. In particular, it is desirable to develop a therapeutic agent that can decrease or mitigate a patient's inflammatory reaction in response to a viral (i.e., RSV) infection, which is a risk factor for the later development of asthma.

Reactive airway disease is a broader (and often times synonymous) characterization for asthma-like symptoms, and is generally characterized by chronic cough, sputum production, wheezing or dyspenea.

Wheezing

Wheezing (also known as sibilant rhonchi) is generally characterized by a noise made by air flowing through narrowed breathing tubes, especially the smaller, tight airways located deep within the lung. It is a common symptom of RSV infection, and secondary RSV conditions such as asthma and bronchiolitis. The clinical importance of wheezing is that it is an indicator of airway narrowing, and it may indicate difficulty breathing.

Wheezing is most obvious when exhaling (breathing out), but may be present during either inspiration (breathing in) or exhalation. Wheezing most often comes from the small bronchial tubes (breathing tubes deep in the chest), but it may originate if larger airways are obstructed.

### Chronic Obstructive Pulmonary Disease (COPD)

Chronic obstructive pulmonary disease (COPD) is a term referring to two lung diseases, chronic bronchitis and emphysema, that are characterized by obstruction to airflow that interferes with normal breathing. Both of these conditions frequently co-exist, hence physicians prefer the term COPD. It does not include other obstructive diseases such as asthma.

Chronic bronchitis is the inflammation and eventual scarring of the lining of the bronchial tubes. When the bronchi are inflamed and/or infected, less air is able to flow to and from the lungs and a heavy mucus or phlegm is coughed up. The condition is defined by the presence of a mucus-producing cough most days of the month, three months of a year for two successive years without other underlying disease to explain the cough.

This inflammation eventually leads to scarring of the lining of the bronchial tubes. Once the bronchial tubes have been irritated over a long period of time, excessive mucus is produced constantly, the lining of the bronchial tubes becomes thickened, an irritating cough develops, and air flow may be hampered, the lungs become scarred. The bronchial tubes then make an ideal breeding place for bacterial infections within the airways, which eventually impedes airflow.

Symptoms of chronic bronchitis include chronic cough, increased mucus, frequent clearing of the throat and shortness of breath. In 2004, an estimated 9 million Americans reported a physician diagnosis of chronic bronchitis. Chronic bronchitis affects people of all ages, but is higher in those over 45 years old.

Smoking is the primary risk factor for COPD. Approximately 80 to 90 percent of COPD deaths are caused by smoking. Other risk factors of COPD include air pollution, second-hand smoke, history of childhood respiratory infections, such as, for example, respiratory syncytial virus (RSV), and heredity.

In 2004, 11.4 million U.S. adults (aged 18 and over) were estimated to have COPD. However, close to 24 million U.S. adults have evidence of impaired lung function, indicating an under diagnosis of COPD. An estimated 638,000 hospital discharges were reported; a discharge rate of 21.8 per 100,000 population. COPD is an important cause of hospitalization in our aged population. Approximately 65% of discharges were in the 65 years and older population in 2004.

In 2004, the cost to the nation for COPD was approximately $37.2 billion, including healthcare expenditures of $20.9 billion in direct health care expenditures, $7.4 billion in indirect morbidity costs and $8.9 billion in indirect mortality costs.

### 3. SUMMARY OF THE INVENTION

The present invention is based, in part, on the development of methods for achieving or inducing a therapeutically effective serum titer of an antibody or fragment thereof that immunospecifically binds to a respiratory syncytial virus (RSV) antigen in a mammal by passive immunization with such an antibody or fragment thereof. The present invention is also based, in part, on the identification of antibodies with higher affinities for a RSV antigen which results in increased efficacy for therapeutic uses such that lower serum titers are therapeutically effective.

In another aspect, the modified antibodies of the invention can be used to treat, manage, and/or ameliorate respiratory conditions, including, but not limited to, long term consequences of RSV infection and/or RSV disease, such as, for example, asthma, wheezing, reactive airway disease (RAD), chronic obstructive pulmonary disease (COPD), or a combination thereof said method comprising administering a therapeutically effective amount of the antibodies of the invention, wherein the management, treatment and/or amelioration is post-infection.

The present invention provides methods of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject comprising administering to said subject one or more antibodies or fragments thereof which immunospecifically bind to one or more RSV antigens with high affinity and/or high avidity. Because a lower serum titer of such antibodies or antibody fragments is therapeutically effective than the effective serum titer of known antibodies, lower doses of said antibodies or antibody fragments can be used to achieve a serum titer effective for the treatment, management, and/or amelioration of respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. The use of lower doses of antibodies or fragments thereof which immunospecifically bind to one or more RSV antigens reduces the likelihood of adverse effects. Further, the high affinity and/or high avidity of the antibodies described herein or fragments thereof enable less frequent administration of said antibodies or antibody fragments than previously thought to be necessary for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof.

In another aspect, the invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering to said subject at least a first dose of a modified antibody of the invention so that said subject has a serum antibody titer of from about 0.1 μg/ml to about 800 μg/ml. In some embodiments, the serum antibody titer is present in the subject for several hours, several days, several weeks, and/or several months. In one embodiment, the first dose of a modified antibody of the invention is administered in a sustained release formulation, and/or by pulmonary or intranasal delivery.

Additionally, the present invention provides an antibody with high affinity and/or high avidity for a RSV antigen (e.g., RSV F antigen) for the treatment and/or amelioration an upper respiratory tract RSV infection (URI) and/or lower respiratory tract RSV infection (LRI) as well as treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, wherein the antibody comprises one or more amino acid modifications in the IgG constant domain, or FcRn-binding fragment thereof (preferably a modified Fc domain or hinge-Fc domain). Such one or more amino acid modifications in the IgG constant domain results in a modified antibody having a modified effector function comprising an altered binding affinity for one or more FcR's as compared to a wild-type antibody without such amino acid modifications.

Contemplated as part of the invention is a modified antibody having a modified Fc domain comprising one or more amino acid substitutions, wherein said amino acid substitutions result in a modified antibody having an increased antibody dependent cell-mediated cytotoxicity (ADCC), compared to the same antibody with a wild-type Fc domain (i.e., without said amino acid substitutions), referred to herein as a “3M” mutation or modified antibody.

Also contemplated as part of the invention is a modified antibody having a modified Fc domain comprising one or more amino acid substitutions, wherein said amino acid substitutions result in a modified antibody having a decreased antibody dependent cell-mediated cytotoxicity (ADCC), compared to the same antibody with a wild-type Fc domain (i.e., without said amino acid substitutions), referred to herein as a “TM” mutation or modified antibody.

It is also contemplated that modified antibodies of the invention include not only those containing amino acid substitutions that either increase or decrease effector functions (i.e., such as ADCC), but also, in addition, amino acid modifications that increases the in vivo half-life of the IgG constant domain, or FcRn-binding fragment thereof. (e.g., Fc or hinge-Fc domain), and any molecule attached thereto, such that the modified antibody of the invention include those with, for example, increased ADCC (3M) combined with increased in vivo half-life in a single modified antibody. Additionally, it is also contemplated that a modified antibody of the invention include those with, for example, decreased ADCC (TM) combined with increased in vivo half-life in a single modified antibody.

The present invention provides methods of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject comprising administering to said subject a therapeutically effective amount of an antibody provided herein (a modified antibody) which immunospecifically binds to a RSV antigen with high affinity and/or high avidity. Because a lower and/or longer-lasting serum titer of the antibodies of the invention will be more effective in the management, treatment and/or amelioration of a RSV infection (e.g., acute RSV disease, or a RSV URI and/or LRI) than the effective serum titer of known antibodies (e.g., palivizumab), lower and/or fewer doses of the antibody can be used to achieve a serum titer effective for the management, treatment and/or amelioration of a RSV infection (e.g., acute RSV disease, or a RSV URI and/or LRI), for example one or more doses per RSV season. The use of lower and/or fewer doses of an antibody of the invention that immunospecifically binds to a RSV antigen reduces the likelihood of adverse effects and are safer for administration to, e.g., infants, over the course of treatment (for example, due to lower serum titer, longer serum half-life and/or better localization to the upper respiratory tract and/or lower respiratory tract as compared to known antibodies (e.g., palivizumab).

In one aspect, the invention provides a method of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, the method comprising administering to a human patient in need thereof a therapeutically effective amount of an antibody described herein (i.e., a modified antibody of the invention), such as a modified antibody that comprises a modified IgG constant domain which include not only those containing amino acid substitutions that either increase or decrease effector functions (i.e., such as ADCC), but also, in addition, amino acid modifications that increases the in vivo half-life of the IgG constant domain, or FcRn-binding fragment thereof (e.g., Fc or hinge-Fc domain), and any molecule attached thereto, such that the modified antibody of the invention include those with, for example, increased ADCC (3M) combined with increased in vivo half-life in a single modified antibody. Additionally, it is also contemplated that a modified antibody of the invention include those with, for example, decreased ADCC (TM) combined with increased in vivo half-life in a single modified antibody. In some embodiments, both upper and lower respiratory tract RSV infections and/or acute RSV disease, can be managed, treated, or ameliorated.

In another aspect, the invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering to said subject a first dose of an antibody of the invention so that said subject has a nasal turbinate and/or nasal secretion antibody concentration of from about 0.01 μg/ml about 2.5 μg/ml. In some embodiments, the nasal turbinate and/or nasal secretion antibody concentration is present in the subject for several hours, several days, several weeks, and/or several months. The first dose of a modified antibody of the invention can be a therapeutically effective dose. In one embodiment, the first dose of an antibody of the invention is administered in a sustained release formulation, and/or by pulmonary or intranasal delivery.

In another aspect, the invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering an effective amount of a modified antibody of the invention, wherein the effective amount results in a reduction in RSV titer as measured in the nasal turbinate and/or nasal secretion and/or bronchial alveolar lavage (BAL) for local responses or measured in serum for a systemic response. The reduction of RSV titer in the above may be as compared to a negative control (such as placebo), as compared to another therapy (including, but not limited to treatment with palivizumab), or as compared to the titer in the patient prior to antibody administration.

In another aspect, the modified antibodies used in accordance with the methods of the invention immunospecifically bind to one or more RSV antigens (e.g., RSV F antigen) and have an association rate constant or kₒₙ rate (antibody (Ab)+antigen (Ag)-kₒₙ->Ab−Ag) of from about 10⁵ M⁻¹ s⁻¹ to about 10¹⁰ M⁻¹ s⁻¹. In some embodiments, the antibody is a high potency antibody having a kₒₙ of from about 10⁵ M⁻¹ s⁻¹ to about 10⁸ M⁻¹ s⁻¹, preferably about 2.5×10⁵ or 5×10⁵ M⁻¹ s⁻¹, and more preferably about 7.5×10⁵ M⁻¹ s⁻¹. Such antibodies may also have a high affinity (e.g., about 10⁹ M⁻¹) or may have a lower affinity. In one embodiment, the antibodies that can be used in accordance with the methods of the invention immunospecifically bind to a RSV antigen (e.g., RSV F antigen) and have a kₒₙ rate that is at least 1.5-fold higher than a known anti-RSV antibody (e.g., palivizumab).

In another aspect, the modified antibodies used in accordance with the methods of the invention immunospecifically bind to one or more RSV antigens (e.g., RSV F antigen) and have a k_{off} rate (Ab−Ag-K_{off}->Ab+Ag) of from less than 5×10⁻¹ s⁻¹ to less than 10×10⁻¹⁰ s⁻¹. In one embodiment, the antibodies used in accordance with the methods of the invention immunospecifically bind to a RSV antigen (e.g., RSV F antigen) and have a k_{off} rate that is at least 1.5-fold lower than a known anti-RSV antibody (e.g., palivizumab).

In another aspect, the modified antibodies that can be used in accordance with the methods of the invention immunospecifically bind to one or more RSV antigens (e.g., RSV F antigen) and have an affinity constant or Kₐ (kₒₙ/k_{off}) of from about 10² M⁻¹ to about 5×10¹⁵ M⁻¹, preferably at least 10⁴ M⁻¹. In some embodiments, the antibody is a high potency antibody having a Kₐ of about 10⁹ M⁻¹, preferably about 10¹⁰ M⁻¹, and more preferably about 10¹¹ M⁻¹.

In another aspect, the modified antibodies of the invention, used in accordance with the methods of the invention immunospecifically bind to one or more RSV antigens (e.g., RSV F antigen) and have a dissociation constant or Kₐ (k_{off}/kₒₙ) of from about 5×10⁻²M to about 5×10⁻¹⁶M.

In another aspect, the modified antibodies that can be used in accordance with the methods of the invention immunospecifically bind to one or more RSV antigens (e.g., RSV F antigen) have a dissociation constant (IQ) of between about 25 pM and about 3000 pM as assessed using an assay described herein or known to one of skill in the art (e.g., a BIAcore assay).

In another aspect, the modified antibodies of the invention, used in accordance with the methods of the invention immunospecifically bind to one or more RSV antigens (e.g., RSV F antigen) and have a median inhibitory concentration (IC₅₀) of about 6 nM to about 0.01 nM in an in vitro microneutralization assay. In certain embodiments, the microneutralization assay is a microneutralization assay described herein (for example, as described in Examples 6.4, 6.8, and 6.18 herein) or as in Johnson et al., 1999, J. Infectious Diseases 180:35-40. In some embodiments, the antibody has an IC₅₀ of less than 3 nM, preferably less than 1 nM in an in vitro microneutralization assay.

In another aspect, the invention provides methods of therapeutically administering one or more antibodies (e.g., a modified antibody) of the invention to a subject (e.g., an infant, an infant born prematurely, an immunocompromised subject, a medical worker). In some embodiments, an antibody of the invention is administered to a subject or human patient so as to prevent a RSV infection from being transmitted from one individual to another, or to lessen the infection that is transmitted. In some embodiments, the subject has been exposed to (and may or may not be asymptomatic), or is likely to be exposed to another individual having RSV infection. Preferably the antibody is administered to the subject intranasally once or more times per day (e.g., one time, two times, four times, etc.) for a period of about one to two weeks after potential or actual exposure to the RSV-infected individual. In certain embodiments, the antibody is administered at a dose of between about 60 mg/kg to about 0.025 mg/kg, and more preferably from about 0.025 mg/kg to 15 mg/kg.

The present invention also provides antibodies or fragments thereof comprising a VH domain having the amino acid sequence of any VH domain listed in Table 1 and compositions comprising said antibodies or antibody fragments for use in treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. The present invention also provides antibodies or fragments thereof comprising one or more VH complementarity determining regions (CDRs) having the amino acid sequence of one or more VH CDRs listed in Table 1 and compositions comprising said antibodies or antibody fragments for use in treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. The present invention also provides antibodies or fragments thereof comprising a VL domain having the amino acid sequence of any VL domain listed in Table 1. The present invention also provides antibodies or fragments thereof comprising one or more VL CDRs having the amino acid sequence of one or more VL CDRs listed in Table 1 and compositions comprising said antibodies or antibody fragments for use in the treatment or amelioration of one or more symptoms and/or long term consequences associated with a RSV infection. The present invention further provides antibodies comprising a VH domain and a VL domain having the amino acid sequence of any VH domain and VL domain listed in Table 1 and compositions comprising said antibodies or antibody fragments for use in the treatment or amelioration of one or more symptoms and/or long term consequences associated with a RSV infection. The present invention further provides antibodies comprising one or more VH CDRs and one or more VL CDRs having the amino acid sequence of one or more VH CDRs and one or more VL CDRs listed in Table 1 and compositions comprising said antibodies or antibody fragments for use in the treatment or amelioration of one or more symptoms and/or long term consequences associated with a RSV infection. In the above embodiments, preferably the antibody binds immunospecifically to a RSV antigen.

In other embodiments, the modified antibodies and methods of the invention encompass the use of antibodies comprising the VH domain and/or VL domain of MEDI-524 (motavizumab). In other embodiments, the methods of the invention encompass the use of antibodies comprising the VH chain and/or VL chain of MEDI-524 (motavizumab). In certain embodiments, the antibody comprises a modified Fc domain, or FcRn-binding fragment thereof, wherein the antibody has increased or decreased affinity for the FcRn receptor relative to the Fc domain of MEDI-524 (motavizumab) that does not comprise a modified Fc domain (i.e., unmodified MEDI-524).

It is also contemplated that the modified antibodies and methods of the invention further modulates a patient's inflammatory response to infection by RSV, as compared to the same antibody without any IgG Fc region modifications. For example, administration of the modified antibodies of the invention to a patient in need thereof will further decrease cytokine release and/or further decrease chemokine release from RSV-infected tissues/cells when compared to the same antibody without any IgG Fc region modifications. It is believed that such a decrease in the pro-inflammatory response in a patient infected with RSV using the modified antibodies of the invention will further mitigate the risk of that patient later developing asthma or other chronic respiratory disease.

The present invention encompasses methods of delivering one or more antibodies or fragments thereof which immunospecifically bind to one or more RSV antigens directly to the site of RSV infection. In particular, the invention encompasses pulmonary delivery of one or more antibodies or fragments thereof which immunospecifically bind to one or more RSV antigens, in order to mitigate long term consequences of RSV infection, such as, for example, chronic obstructive pulmonary disease (COPD). The improved methods of delivering of one or more antibodies or fragments thereof which immunospecifically bind to one or more RSV antigens reduce the dosage and/or frequency of administration of said antibodies or antibody fragments to a subject.

3.1 TERMINOLOGY

The terms “antibodies that immunospecifically bind to a RSV antigen,” “anti-RSV antibodies,” “modified antibody” and analogous terms as used herein refer to Fc modified antibodies (i.e., antibodies that comprise a modified IgG (e.g., IgG1) constant domain, or FcRn-binding fragment thereof (e.g., the Fc-domain or hinge-Fc domain)), that specifically bind to a RSV polypeptide. An antibody or a fragment thereof that immunospecifically binds to a RSV antigen may be cross-reactive with related antigens. Preferably, an antibody or a fragment thereof that immunospecifically binds to a RSV antigen does not cross-react with other antigens. An antibody or a fragment thereof that immunospecifically binds to a RSV antigen can be identified, for example, by immunoassays, BIAcore, or other techniques known to those of skill in the art. An Fc modified antibody or a fragment thereof binds specifically to a RSV antigen when it binds to a RSV antigen with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to a RSV antigen (preferably, a RSV F antigen) (e.g., one or more complementarity determining regions (CDRs) of an anti-RSV antibody). The antibodies of the invention can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. In other embodiments, modified antibodies of the invention are IgG antibodies, or a class (e.g., human IgG1) or subclass thereof.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “effective neutralizing titer” as used herein refers to the amount of antibody which corresponds to the amount present in the serum of animals (human or cotton rat) that has been shown to be either clinically efficacious (in humans) or to reduce virus by 99% in, for example, cotton rats. The 99% reduction is defined by a specific challenge of, e.g., 10³ pfu, 10⁴ pfu, 10⁵ pfu, 10⁶ pfu, 10⁷ pfu, 10⁸ pfu, or 10⁹ pfu of RSV.

The term “elderly” as used herein refers to a human subject who is age 65 or older.

The term “FcRn receptor” or “FcRn” as used herein refers to an Fc receptor (“n” indicates neonatal) which is known to be involved in transfer of maternal IgGs to a fetus through the human or primate placenta, or yolk sac (rabbits) and to a neonate from the colostrum through the small intestine. It is also known that FcRn is involved in the maintenance of constant serum IgG levels by binding the IgG molecules and recycling them into the serum. The binding of FcRn to IgG molecules is pH-dependent with optimum binding at pH 6.0. The amino acid sequences of human FcRn and murine FcRn are indicated by SEQ ID NO:337 and SEQ ID NO:338, respectively.

The term “fusion protein” as used herein refers to a polypeptide that comprises an amino acid sequence of an antibody and an amino acid sequence of a heterologous polypeptide or protein (i.e., a polypeptide or protein not normally a part of the antibody (e.g., a non-anti-RSV antigen antibody)).

The term “high potency” as used herein refers to antibodies that exhibit high potency as determined in various assays for biological activity (e.g., neutralization of RSV) such as those described herein. For example, high potency antibodies of the invention have an IC₅₀ value less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, as measured by a microneutralization assay. In certain embodiments, the microneutralization assay is a microneutralization assay described herein or as in Johnson et al., 1999, J. Infectious Diseases 180:35-40. Further, high potency antibodies of the invention result in at least a 75%, preferably at least a 95% and more preferably a 99% lower RSV titer in a cotton rat 5 days after challenge with 10⁵ pfu relative to a cotton rat not administered said antibodies. In certain embodiments of the invention, high potency antibodies of the present invention exhibit a high affinity and/or high avidity for one or more RSV antigens (e.g., antibodies having an affinity of at least 2×10⁸ M⁻¹, preferably between 2×10⁸M⁻¹ and 5×10¹²M⁻¹, such as at least 2.5×10⁸M⁻¹, at least 5×10⁸M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹² M⁻¹ for one or more RSV antigens).

The term “human infant” as used herein refers to a human less than 24 months, preferably less than 16 months, less than 12 months, less than 6 months, less than 3 months, less than 2 months, or less than 1 month of age.

The term “human infant born prematurely” as used herein refers to a human born at less than 40 weeks gestational age, preferably less than 35 weeks gestational age, wherein the infant is less than 6 months old, preferably less than 3 months old, more preferably less than 2 months old, and most preferably less than 1 month old.

The terms “IgG Fc region,” “Fc region,” “Fc domain,” “Fc fragment” and other analogous terms as used herein refers the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but contains the carbohydrate moiety and the binding sites for complement and Fc receptors, including the FcRn receptor (see below). For example, an Fc fragment contains the entire second constant domain CH2 (residues 231-340 of human IgG1, see SEQ ID NO:339) and the third constant domain CH3 (residues 341-447 of human IgG1, see, SEQ ID NO:340). All numbering used herein is according to the EU Index (Kabat et al. (1991) Sequences of proteins of immunological interest. (U.S. Department of Health and Human Services, Washington, D.C.) 5^{th} ed.), unless otherwise indicated.

The term “IgG hinge-Fc region” or “hinge-Fc fragment” as used herein refers to a region of an IgG molecule consisting of the Fc region (residues 231-447) and a hinge region (residues 216-230; e.g., SEQ ID NO:341) extending from the N-terminus of the Fc region, according to the EU Index (Kabat et al. (1991) Sequences of proteins of immunological interest. (U.S. Department of Health and Human Services, Washington, D.C.) 5^{th} ed.). An example of the amino acid sequence of the human IgG1 hinge-Fc region is SEQ ID NO:342.

As used herein, the terms “infection” and “RSV infection” refer to all stages of RSV's life cycle in a host (including, but not limited to the invasion by and replication of RSV in a cell or body tissue), as well as the pathological state resulting from the invasion by and replication of a RSV. The invasion by and multiplication of a RSV includes, but is not limited to, the following steps: the docking of the RSV particle to a cell, fusion of a virus with a cell membrane, the introduction of viral genetic information into a cell, the expression of RSV proteins, the production of new RSV particles and the release of RSV particles from a cell. An RSV infection may be an upper respiratory tract RSV infection (URI), a lower respiratory tract RSV infection (LRI), or a combination thereof. In specific embodiments, the pathological state resulting from the invasion by and replication of a RSV is an acute RSV disease. The term “acute RSV disease” as used herein refers to clinically significant disease in the lungs or lower respiratory tract as a result of an RSV infection, which can manifest as pneumonia and/or bronchiolitis, where such symptoms may include hypoxia, apnea, respiratory distress, rapid breathing, wheezing, cyanosis, etc. Acute RSV disease requires an affected individual to obtain medical intervention, such as hospitalization, administration of oxygen, intubation and/or ventilation.

The term “in vivo half-life” as used herein refers to a biological half-life of a particular type of IgG molecule or its fragments containing FcRn-binding sites in the circulation of a given animal and is represented by a time required for half the quantity administered in the animal to be cleared from the circulation and/or other tissues in the animal. When a clearance curve of a given IgG is constructed as a function of time, the curve is usually biphasic with a rapid α-phase which represents an equilibration of the injected IgG molecules between the intra- and extra-vascular space and which is, in part, determined by the size of molecules, and a longer β-phase which represents the catabolism of the IgG molecules in the intravascular space. The term “in vivo half-life” practically corresponds to the half-life of the IgG molecules in the β-phase. As used herein, “increased in vivo serum half-life” or “extended in vivo serum half-life” of an antibody that comprises a modified IgG constant domain, or FcRn-binding fragment thereof (preferably the Fc domain or the hinge-Fc domain), refers to an increase in in vivo serum half-life of the antibody as compared to an antibody that does not comprise a modified IgG constant domain, or FcRn-binding fragment thereof (e.g., as compared to an the antibody that does not comprise the one or more modifications in the constant domain, or FcRn-binding fragment thereof (i.e., an unmodified antibody), or as compared to another RSV antibody, such as palivizumab).

The term “lower respiratory” tract refers to the major passages and structures of the lower respiratory tract including the windpipe (trachea) and the lungs, including the bronchi, bronchioles, and alveoli of the lungs.

As used herein, the term “MEDI-524” is an unmodified anti-RSV monoclonal antibody (motavizumab) described in Wu et al., J. Mol. Biol. 368, 652-665 (2007), herein incorporated by reference in its entirety.

As used herein, the term “modified antibody” is also synonymous with “Fc modified antibody” encompasses any antibody described herein that comprises one or more “modifications” to the amino acid residues at given positions of the antibody constant domain (preferably an IgG and more preferably an IgG1 constant domain), or FcRn-binding fragment thereof wherein the antibody can have a modified effector function (i.e., ADCC) and, in combination, has an increased in vivo half-life as compared to the same antibody that does not comprise one or more modifications in the IgG constant domain, or FcRn-binding fragment thereof, as a result of, e.g., one or more modifications in amino acid residues identified to be involved in the interaction between the constant domain, or FcRn-binding fragment thereof (preferably, an Fc domain or hinge-Fc domain), of said antibodies and the Fc Receptor neonate (FcRn). The term “modified antibody” or “Fc modified antibody” also encompasses antibodies that naturally comprise one or more of the recited residues at the indicated positions (e.g., the residues are already present in the recited position in the molecule without modification). Numbering of constant domain positions is according to the EU Index (Kabat et al. (1991) Sequences of proteins of immunological interest. (U.S. Department of Health and Human Services, Washington, D.C.) 5^{th} ed.). As used herein, a “modified antibody” or “Fc modified antibody” may or may not be a high potency, high affinity and/or high avidity modified antibody. In certain embodiments, the modified antibody is a high potency antibody, and in other embodiments, a high potency as well as a high affinity modified antibody.

As used herein, one or more “modifications to the amino acid residues” in the context of a constant domain, or FcR-binding fragment thereof, of an antibody of the invention refers to any mutation, substitution, insertion or deletion of one or more amino acid residues of the sequence of the constant domain, or FcR-binding fragment thereof (preferably, Fc domain or hinge-Fc domain) of the antibody. Preferably, the one or more modifications are substitutions. In one embodiment, the one or more amino acid substitutions are: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat. In another embodiment, the one or more amino acid substitutions are: 239D, 330L, and 332E, wherein the numbering system is that of the EU index as set forth in Kabat. Such Fc domain amino acid substitutions encompass an increase in ADCC (3M) if compared to the same antibody without said amino acid substitutions. In another embodiment, the one or more amino acid substitutions is selected from the group consisting of 233P, 234F, 234V, 235A, 235E, 265A, 327G, 330S, and 331S, wherein the numbering system is that of the EU index as set forth in Kabat. In another embodiment, the one or more amino acid substitutions is selected from the group consisting of: 234F, 235E, and 331S, wherein the numbering system is that of the EU index as set forth in Kabat. Such Fc domain amino acid substitutions encompass a decrease in ADCC (TM) if compared to the same antibody without said amino acid substitutions. In another embodiment, the one or more amino acid modifications are, in addition to those described for 3M and TM, in combination with those at positions 251-256, 285-290, 308-314, 385-389, and 428-436, with numbering according to the EU Index as in Kabat et al., supra. Such Fc domain combination amino acid substitutions encompass a modified antibody having either an increase in ADCC (3M) with an increase in in vivo half life, or a modified antibody having a decrease in ADCC (TM) with an increase in in vivo half life, if both are compared to the same antibody without said amino acid substitutions. In certain other embodiments, an IgG constant domain comprises a Y at position 252 (252Y), a T at position 254 (254T), and/or an E at position 256 (256E), wherein the numbering system is that of the EU index as set forth in Kabat. Such a combination of amino acid mutations serve to increase serum half-life of antibodies of the invention.

The term “multiplicity of infection” (M.O.I) as used herein is a way of quantifying the average number of RSV virus that infects a single cell, tissue or patient. In one embodiment, patients having an RSV infection considered to be a clinical RSV infection, have a measured RSV M.O.I. ranging from about 0.001 to about 0.1. In yet another embodiment, patients having an RSV infection considered to be a clinical RSV infection, have a measured RSV M.O.I. of about 0.1 or of about 0.01.

The term “nursing home” as used herein means a human patient who is living in a nursing home or skilled nursing facility (SNF) or place of communal residence for people who require constant nursing care and have significant deficiencies with activities of daily living. Residents may include, for example, the elderly and younger adults with physical disabilities.

The term “pharmaceutically acceptable” as used herein means being approved by a regulatory agency of the Federal or a state government, or listed in the U.S. Pharmacopia, European Pharmacopia or other generally recognized pharmacopia for use in animals, and more particularly in humans.

The term “RSV antigen” refers to a RSV polypeptide to which an antibody immunospecifically binds. A RSV antigen also refers to an analog or derivative of a RSV polypeptide or fragment thereof to which an antibody immunospecifically binds. In some embodiments, a RSV antigen is a RSV F antigen, RSV G antigen or a RSV SH antigen.

The term “serum titer” as used herein refers to an average serum titer in a population of least 10, preferably at least 20, and most preferably at least 40 subjects up to about 100, 1000 or more.

As used herein, the term “side effects” encompasses unwanted and adverse effects of a therapy (e.g., a therapeutic agent). Unwanted effects are not necessarily adverse. An adverse effect from a therapy (e.g., a therapeutic agent) might be harmful or uncomfortable or risky. Examples of side effects include, but are not limited to, URI, rhinitis, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (58^{th} ed., 2004).

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human. In one embodiment, the subject is a mammal, preferably a human, with a RSV infection (e.g., acute RSV disease, or a RSV URI and/or LRI). In another embodiment, the subject is a mammal, preferably a human, at risk of developing a RSV infection (e.g., acute RSV disease, or a RSV URI and/or LRI) (e.g., an immunocompromised or immunosuppressed mammal, or a genetically predisposed mammal). In one embodiment, the subject is a human with a respiratory condition (including, but not limited to asthma, wheezing or RAD) that stems from, is caused by or associated with a RSV infection. In some embodiments, the subject is 0-5 years old or is a human infant, preferably age 0-2 years old (e.g., 0-12 months old). In other embodiments, the subject is an elderly subject.

In certain embodiments of the invention, a “therapeutically effective serum titer” is the serum titer in a subject, preferably a human that reduces the severity, the duration and/or the symptoms associated with a RSV infection (e.g., acute RSV disease or RSV URI and/or LRI) in said subject. Preferably, the therapeutically effective serum titer reduces the severity, the duration and/or the number symptoms associated with a RSV infection (e.g., acute RSV disease or RSV URI and/or LRI) in humans with the greatest probability of complications resulting from the infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “therapeutically effective serum titer” is the serum titer in a cotton rat that results in a RSV titer 5 days after challenge with 10⁵ pfu that is 99% lower than the RSV titer 5 days after challenge with 10⁵ pfu of RSV in a cotton rat not administered an antibody that immunospecifically binds to a RSV antigen. In some embodiments, the therapeutically effective amount of an antibody of the invention is about 0.025 mg/kg, about 0.05 mg/kg, about 0.10 mg/kg, about 0.20 mg/kg, about 0.40 mg/kg, about 0.80 mg/kg, about 1.0 mg/kg, about 1.5 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg or about 60 mg/kg. In one embodiment, a therapeutically effective amount of an antibody of the invention is about 15 mg of the antibody per kg of body weight of the subject.

As used herein, the terms “treat,” “treatment” and “treating” refer to the administration post-infection to result in the reduction or amelioration of the progression, severity, and/or duration of a RSV infection (e.g., acute RSV disease, or a RSV URI and/or LRI), or a symptom or respiratory condition relating thereto (including, but not limited to, asthma, wheezing, RAD, or a combination thereof) resulting from the administration of one or more therapies (including, but not limited to, the administration of one or more therapeutic agents, such as an antibody of the invention). In specific embodiments, such terms refer to the reduction or inhibition of the replication of RSV, the inhibition or reduction in the spread of RSV to other tissues or subjects (e.g., the spread to the lower respiratory tract), the inhibition or reduction of infection of a cell with a RSV, the inhibition or reduction of acute RSV disease, the inhibition or reduction of a respiratory condition caused by or associated with RSV infection (e.g., asthma, wheezing and/or RAD), and/or the inhibition or reduction of one or more symptoms associated with a RSV infection.

The term “upper respiratory” tract refers to the major passages and structures of the upper respiratory tract including the nose or nostrils, nasal cavity, mouth, throat (pharynx), and voice box (larynx).

### 4. DESCRIPTION OF THE FIGURES

FIG. 1 shows MEDI-524 added 1 hour or 12 hours post-infection to RSV-infected epithelial Hep-2 cells (RSV-524) and then assayed for the presence of IL-6 or IL-8 secreted by the RSV-infected Hep-2 cells. The control is MEDI-507, an anti-CD2 antibody considered irrelevant (RSV-507). MEDI-524 added 1 hour post-RSV infection demonstrates a greater decrease in IL-6 and IL-8 secretion from RSV-infected cells than at 12 hours post-infection.

FIG. 2 shows MEDI-524 added 1 hour or 12 hours post-infection to RSV-infected epithelial Hep-2 cells (RSV-524) and then assayed for the presence of IL-12p70 or TNF-alpha secreted by the RSV-infected Hep-2 cells. The control is MEDI-507, an anti-CD2 antibody considered irrelevant (RSV-507). MEDI-524 added 1 hour post-RSV infection demonstrates a greater decrease in IL-12p70 and TNF-alpha secretion from RSV-infected cells than at 12 hours post-infection.

FIG. 3 shows MEDI-524 mediated chemokine release of MIP-1b and MCP-1 from activated macrophages in co-culture with RSV-infected Hep-2 cells.

FIG. 4 shows MEDI-524 mediated chemokine release of IP-10 and eotaxin-3 from activated macrophages in co-culture with RSV-infected Hep-2 cells.

FIG. 5 shows MEDI-524 mediated THP-1 activation by FACS analysis. MEDI-524 or control antibody MEDI-507 were added post-infection, to DiD-stained RSV-infected Hep-2 cells mixed with IFN-γ-activated THP-1 cells and analyzed for HLADR-PE for THP-1 cells on the x-axis and DiD-APC for Hep-2 cells on the y-axis. MEDI-524 can mediate monocyte phagocytosis of RSV-infected cells.

FIG. 6 shows MEDI-524 and MEDI-524 3M (having the amino acid mutations 239D, 330L, 332E as in Kabat numbering) mediated antibody-dependent cell-mediated cytotoxicity (ADCC). RSV-infected Hep-2 cells were mixed with NK effector cells, then either MEDI-524 or MEDI-524 3M were added. Cytotoxicity was measured in an ADCC assay, LDH release assay.

FIG. 7 shows the therapeutic efficacy of MEDI-524 TM (having the amino acid mutations 234F, 235E, 331S as in Kabat numbering) over MEDI-524 on reduction of viral titers in cotton rat lung homogenates, using a viral plaque assay to measure amounts of viral titers. Groups of four animals each were injected intraperitoneally with either motavizumab (MEDI-524), an ADCC enhanced variant (MEDI-524-3M) or a ADCC deficient variant (MEDI-524-TM) at a concentration of 7 mg/kg at different time points (24 hrs prior infection or 72 hrs post infection). One group of animals was left untreated and received only virus (Naïve infected) and one group was left untreated and uninfected (naïve uninfected). At time point 0, all animals were infected intranasally with 10⁵ pfu and four days after infection all animals were sacrificed and analyzed for viral titers. Shown are viral titers of the lung (log₁₀ pfu/g [mean±standard error]).

FIG. 8 shows post-RSV infection addition of motavizumab or MEDI-524 at 1 hour led to a decrease in PD-L1 expression on A549 cells.

FIG. 9 shows post-RSV infection addition of motavizumab or MEDI-524 at 1 hour, 6 hours or 12 hours, all led to a decrease in ICAM-1 expression on A549 cells.

FIG. 10 shows post-RSV infection addition of motavizumab or MEDI-524 at 1 hour, 6 hours or 12 hours, all led to a decrease (in fold induction) in cellular apoptosis (as measured by caspase 3/7 activity) of A549 cells.

FIG. 11 shows the percent of floating A549 cells after RSV infection and the percent with motavizumab or MEDI-524 1 hour, 6 hours or 12 hours post-RSV infection of A549 cells.

FIG. 12 shows the addition of motavizumab or MEDI-524 post RSV infection, which leads to a decrease of RSV release into the cell culture supernatant of both HEp-2 cells and A549 cells.

### 5. DETAILED DESCRIPTION OF THE INVENTION

The interaction of antibodies and antibody-antigen complexes with cells of the immune system effects a variety of responses, including antibody-dependent cell-mediated cytotoxicity (ADCC) and complement dependent cytotoxicity (CDC) (reviewed in Daëron, Annu. Rev. Immunol. 15:203-234 (1997); Ward and Ghetie, Therapeutic Immunol. 2:77-94 (1995); as well as Ravetch and Kinet, Annu. Rev. Immunol. 9:457-492 (1991)). ADCC refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991).

Several antibody effector functions are mediated by Fc receptors (FcRs), which bind the Fc region of an antibody. FcRs are defined by their specificity for immunoglobulin isotypes; Fc receptors for IgG antibodies are referred to as FcγR, for IgE as FcεR, for IgA as Fc+R and so on. Three subclasses of FcγR have been identified: FcγRI (CD64), FcγRII (CD32) and FcγRIII (CD16). These different FcR subtypes are expressed on different cell types (reviewed in Ravetch and Kinet, Annu. Rev. Immunol. 9:457-492 (1991)). For example, in humans, FcγRIIIB is found only on neutrophils, whereas FcγRIIIA is found on macrophages, monocytes, natural killer (NK) cells, and a subpopulation of T-cells. Notably, FcγRIIIA is the only FcR present on NK cells, one of the cell types implicated in ADCC.

Additionally, the present invention provides an antibody with high affinity and/or high avidity for a RSV antigen (e.g., RSV F antigen) for the treatment and/or amelioration an upper respiratory tract RSV infection (URI) and/or lower respiratory tract RSV infection (LRI) as well as treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, wherein the antibody comprises one or more amino acid modifications in the IgG constant domain, or FcRn-binding fragment thereof (preferably a modified Fc domain or hinge-Fc domain) that increases the in vivo half-life of the IgG constant domain, or FcRn-binding fragment thereof (e.g., Fc or hinge-Fc domain), and any molecule attached thereto, and increases the affinity of the IgG, or FcRn-binding fragment thereof containing the modified region, for FcRn (i.e., a “modified antibody”). The amino acid modifications may be any modification of a residue (and, in some embodiments, the residue at a particular position is not modified but already has the desired residue), preferably at one or more of residues 251-256, 285-290, 308-314, 385-389, and 428-436, wherein the modification increases the affinity of the IgG, or FcRn-binding fragment thereof containing the modified region, for FcRn. In other embodiments, the antibody comprises a tyrosine at position 252 (252Y), a threonine at position 254 (254T), and/or a glutamic acid at position 256 (256E) (numbering of the constant domain according to the EU index in Kabat et al. (1991). Sequences of proteins of immunological interest. (U.S. Department of Health and Human Services, Washington, D.C.) 5^{th} ed. (“Kabat et al.”)) in the constant domain, or FcRn-binding fragment thereof. In other embodiments, the antibodies comprise 252Y, 254T, and 256E (see EU index in Kabat et al., supra) in the constant domain, or FcRn-binding fragment thereof (hereafter “YTE”).

The present invention provides methods of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject comprising administering to said subject an effective amount of an antibody provided herein (a modified antibody) which immunospecifically binds to a RSV antigen with high affinity and/or high avidity. Because a lower and/or longer-lasting serum titer of the antibodies of the invention will be more effective in the management, treatment and/or amelioration of a RSV infection (e.g., acute RSV disease, or a RSV URI and/or LRI) than the effective serum titer of known antibodies (e.g., palivizumab), lower and/or fewer doses of the antibody can be used to achieve a serum titer effective for the management, treatment and/or amelioration of a RSV infection (e.g., acute RSV disease, or a RSV URI and/or LRI), for example one or more doses per RSV season. The use of lower and/or fewer doses of an antibody of the invention that immunospecifically binds to a RSV antigen reduces the likelihood of adverse effects and are safer for administration to, e.g., infants, over the course of treatment (for example, due to lower serum titer, longer serum half-life and/or better localization to the upper respiratory tract and/or lower respiratory tract as compared to known antibodies (e.g., palivizumab). In certain embodiments, an antibody is administered once or twice per RSV season.

Accordingly, the invention provides antibodies, and methods of using the antibodies thereof, having an increased potency and/or that have increased affinity and/or increased avidity for a RSV antigen (preferably RSV F antigen) as compared to a known RSV antibody (e.g., palivizumab). In some embodiments, the antibody comprises a modified IgG constant domain, or FcRn-binding fragment thereof (preferably, Fc domain or hinge-Fc domain), which results in increased in vivo serum half-life, as compared to, for example, antibodies that do not comprise a modified IgG constant domain, or FcRn-binding fragment thereof (e.g., as compared to the same antibody that does not comprise one or more modifications in the IgG constant domain, or Fc-binding fragment thereof (i.e., the same, unmodified antibody), or as compared to another RSV antibody, such as palivizumab). In some embodiments, the antibodies are administered to a subject, wherein the subject is human subject.

In a specific embodiment, the invention provides a method of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, the method comprising administering to a subject an effective amount of an antibody described herein, for example a modified antibody (i.e., an antibody of the invention). In another embodiment, the invention provides a method of managing, treating and/or ameliorating an acute RSV disease, or progression to an acute RSV disease, the method comprising administering to a subject an effective amount of an antibody of the invention. In some embodiments, the symptom or respiratory condition relating to the RSV infection is asthma, wheezing, RAD, nasal congestion, nasal flaring, cough, tachypnea (rapid coughing), shortness of breath, fever, croupy cough, or a combination thereof. In some embodiments, both upper and lower respiratory tract RSV infections are prevented, treated, managed, and/or ameliorated. In other embodiments, the progression from an upper respiratory tract infection to a lower respiratory tract infection is prevented, treated, managed, and/or ameliorated. In other embodiments, acute RSV disease, or the progression to an acute RSV disease, is prevented, treated, managed, and/or ameliorated.

In a specific embodiment, the invention provides a method of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, the method comprising administering to a subject an effective amount of an antibody of the invention. In another embodiment, the invention provides a method of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, the method comprising administering to a subject an effective amount of an antibody of the invention and an effective amount of a therapy other than an antibody of the invention. Preferably, such a therapy is useful in the management, treatment and/or amelioration of a RSV infection (preferably an acute RSV disease, or a RSV URI and/or LRI). In another embodiment, the treated, managed and/or ameliorated in accordance with the methods of the invention stems from, is caused by or is associated with a RSV infection, preferably a RSV URI and/or LRI.

The present invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering to said subject at least a first dose of an antibody of the invention so that said subject has a serum antibody titer of from about 0.1 μg/ml to about 800 μg/ml, such as between 0.1 μg/ml and 500 μg/ml, 0.1 μg/ml and 250 μg/ml, 0.1 μg/ml and 100 μg/ml, 0.1 μg/ml and 50 μg/ml, 0.1 μg/ml and 25 μg/ml or 0.1 μg/ml and 10 μg/ml. In certain embodiments, the serum antibody titer is at least 0.1 μg/ml, at least 0.2 μg/ml, at least 0.4 μg/ml, at least 0.6 μg/ml, at least 0.8 μg/ml, at least 1 μg/ml, at least 1.5 μg/ml, at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml, at least 105 μg/ml, at least 110 μg/ml, at least 115 μg/ml, at least 120 μg/ml, at least 125 μg/ml, at least 130 μg/ml, at least 135 μg/ml, at least 140 μg/ml, at least 145 μg/ml, at least 150 μg/ml, at least 155 μg/ml, at least 160 μg/ml, at least 165 μg/ml, at least 170 μg/ml, at least 175 μg/ml, at least 180 μg/ml, at least 185 μg/ml, at least 190 μg/ml, at least 195 μg/ml, or at least 200 μg/ml, at least 250 μg/ml, at least 300 μg/ml, at least 350 μg/ml, at least 400 μg/ml, at least 450 μg/ml, at least 500 μg/ml, at least 550 μg/ml, at least 600 μg/ml, at least 650 μg/ml, at least 700 μg/ml, at least 750 μg/ml, or at least 800 μg/ml. In one embodiment, a therapeutically effective dose results in a serum antibody titer of approximately 75 μg/ml or less, approximately 60 μg/ml or less, resulting in a serum antibody titer of approximately 50 μg/ml or less, approximately 45 μg/ml or less, approximately 30 μg/ml or less, and preferably at least 2 μg/ml, more preferably at least 4 μg/ml, and most preferably at least 6 μg/ml.

In some embodiments the aforementioned serum antibody concentrations are present in the subject at about or for about 12 to 24 hours after the administration of the first dose of the antibody of the invention and prior to the optional administration of a subsequent dose. In some embodiments, the aforementioned serum antibody concentrations are present for a certain amount of days after the administration of the first dose of the antibody and prior to the optional administration of a subsequent dose, wherein said certain number of days is from about 20 days to about 180 days (or longer), such as between 20 days and 90 day, 20 days and 60 days, or 20 days and 30 days, and in certain embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or longer. In certain embodiments, the first dose of the antibody resulting in the aforementioned serum antibody concentrations is about 60 mg/kg or less, about 50 mg/kg or less, about 45 mg/kg or less, about 40 mg/kg or less, about 30 mg/kg or less, about 20 mg/kg or less, about 15 mg/kg or less, about 10 mg/kg or less, about 5 mg/kg or less, about 4 mg/kg or less, about 3 mg/kg, about 2 mg/kg or less, about 1.5 mg/kg or less, about 1.0 mg/kg or less, about 0.80 mg/kg or less, about 0.40 mg/kg or less, about 0.20 mg/kg or less, about 0.10 mg/kg or less, about 0.05 mg/kg or less, or about 0.025 mg/kg or less. In some embodiments, the first dose of an antibody of the invention is a therapeutically effective dose that results in any one of the aforementioned serum antibody concentrations. In one embodiment, the first dose of an antibody of the invention is administered in a sustained release formulation and/or by intranasal or pulmonary delivery.

The present invention also provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering to said subject a first dose of an antibody of the invention so that said subject has a reduced RSV viral lung titer and/or RSV viral sputum titer (as determined using methods well known to those skilled in the art) as compared to a negative control, for example a subject receiving a placebo, as compared to the tiers in a subject prior to administration of the first dose of an antibody of the invention, or as compared to a subject receiving another RSV antibody (e.g., palivizumab). In embodiments, wherein the antibody is a modified antibody of the invention, the reduced RSV viral lung tier and/or RSV viral sputum titer may further be compared to a subject receiving the same antibody without the modifications in the IgG constant domain.

The present invention also provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering to said subject a first dose of an antibody of the invention so that said subject has a nasal turbinate and/or nasal secretion and/or bronchial alveolar lavaged (BAL) antibody concentration of from about 0.01 μg/ml to about 2.5 μg/ml (or more). In certain embodiments, the nasal turbinate and/or nasal secretion and/or BAL antibody concentration is at least 0.01 μg/ml, at least 0.011 μg/ml, at least 0.012 μg/ml, at least 0.013 μg/ml, at least 0.014 μg/ml, at least 0.015 μg/ml, at least 0.016 μg/ml, at least 0.017 μg/ml, at least 0.018 μg/ml, at least 0.019 μg/ml, at least 0.02 μg/ml, at least 0.025 μg/ml, at least 0.03 μg/ml, at least 0.035 μg/ml, at least 0.04 μg/ml, at least 0.05 μg/ml, at least 0.06 μg/ml, at least 0.07 μg/ml, at least 0.08 μg/ml, at least 0.09 μg/ml, at least 0.1 μg/ml, at least 0.11 μg/ml, at least 0.115 μg/ml, at least 0.12 μg/ml, at least 0.125 μg/ml, at least 0.13 μg/ml, at least 0.135 μg/ml, at least 0.14 μg/ml, at least 0.145 μg/ml, at least 0.15 μg/ml, at least 0.155 μg/ml, at least 0.16 μg/ml, at least 0.165 μg/ml, at least 0.17 μg/ml, at least 0.175 μg/ml, at least 0.18 μg/ml, at least 0.185 μg/ml, at least 0.19 μg/ml, at least 0.195 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, at least 1.5 μg/ml, at least 1.6 μg/ml, at least 1.7 μg/ml, at least 1.8 μg/ml, at least 1.9 μg/ml, at least 2.0 μg/ml, at least 2.1 μg/ml, at least 2.2 μg/ml, at least 2.3 μg/ml, at least 2.4 μg/ml, at least 2.5 μg/ml or more.

In some embodiments the aforementioned nasal turbinate and/or nasal secretion antibody concentrations are present in the subject at about or for about 12 to 24 hours after the administration of the first dose of the antibody of the invention and prior to the optional administration of a subsequent dose. In some embodiments, the aforementioned nasal turbinate and/or nasal secretion and/or BAL antibody concentrations are present for a certain amount of days after the administration of the first dose of the antibody and prior to the optional administration of a subsequent dose, wherein said certain number of days is from about 20 days to about 180 days (or more), and in certain embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or more. In certain embodiments, the first dose of the antibody resulting in the aforementioned nasal turbinate and/or nasal secretion and/or BAL antibody concentrations is about 60 mg/kg or less, about 50 mg/kg or less, about 45 mg/kg or less, about 40 mg/kg or less, about 30 mg/kg or less, about 20 mg/kg or less, about 15 mg/kg or less, about 10 mg/kg or less, about 5 mg/kg or less, about 4 mg/kg or less, about 3 mg/kg, about 2 mg/kg or less, about 1.5 mg/kg or less, about 1.0 mg/kg or less, about 0.80 mg/kg or less, about 0.40 mg/kg or less, about 0.20 mg/kg or less, about 0.10 mg/kg or less, about 0.05 mg/kg or less, or about 0.025 mg/kg or less. In some embodiments, the first dose of an antibody of the invention is a therapeutically effective dose that results in any one of the aforementioned nasal turbinate and/or nasal secretion and/or BAL antibody concentrations. In one embodiment, the first dose of an antibody of the invention is administered in a sustained release formulation and/or by intranasal and/or pulmonary delivery.

In specific embodiments, the present invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof in a subject, said methods comprising administering an effective amount of an antibody of the invention, wherein the effective amount results in a reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120 fold, about 125-fold or higher in RSV titer in the nasal turbinate and/or nasal secretion and/or BAL. The fold-reduction in RSV titer in the nasal turbinate and/or nasal secretion and/or BAL may be as compared to a negative control (such as placebo), as compared to another therapy (including, but not limited to treatment with palivizumab), as compared to the titer in the patient prior to antibody administration or, in the case of modified antibodies, as compared to the same unmodified antibody (e.g., the same antibody prior to constant region modification).

The present invention provides methods of neutralizing RSV in the upper and/or lower respiratory tract or in the middle ear using an antibody of the invention to achieve a therapeutically effective serum titer, wherein said effective serum titer is less than 30 μg/ml (and is preferably about 2 μg/ml, more preferably about 4 μg/ml, and most preferably about 6 μg/ml) for about 20, 25, 30, 35, 40, 45, 60, 75, 90, 105, 120, 135, 150, 165, 180 or more days after administration without any other dosage administration. The antibody of the invention may or may not comprise a modified IgG (e.g., IgG1) constant domain, or FcRn-binding fragment thereof (e.g., Fc or hinge-Fc domain) as described herein.

In other embodiments, the antibodies used in accordance with the methods of the invention have a high affinity for RSV antigen. In one embodiment, the antibodies used in accordance with the methods of the invention have a higher affinity for a RSV antigen (e.g., RSV F antigen) than known antibodies, (e.g., palivizumab or other wild-type antibodies). The antibody used in accordance with the methods of the invention may or may not comprise a modified IgG (e.g., IgG1) constant domain, or FcRn-binding fragment thereof (e.g., Fc or hinge-Fc domain). In certain embodiments, the antibody is a modified antibody, and preferably the IgG constant domain comprises the extended serum half-life YTE modification (e.g., MEDI-524 YTE). In a specific embodiment, the antibodies used in accordance with the methods of the invention have a 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV antigen than a known anti-RSV antibody as assessed by techniques described herein or known to one of skill in the art (e.g., a BIAcore assay or Kinexa assay). In a more specific embodiment, the antibodies used in accordance with the methods of the invention have a 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV F antigen than palivizumab as assessed by techniques described herein or known to one of skill in the art (e.g., a BIAcore assay or Kinexa assay). In another embodiment, the antibodies used in accordance with the methods of the invention have a 65-fold, preferably 70-fold, or higher affinity for a RSV F antigen than palivizumab as assessed by techniques described herein or known to one of skill in the art (e.g., a BIAcore assay or Kinexa assay). In accordance with these embodiments, the affinity of the antibodies is, in one embodiment, assessed by a BIAcore assay. In another embodiment, the affinity of the antibodies is assessed by a Kinexa assay.

In one embodiment, the antibodies used in accordance with the methods of the invention immunospecifically bind to one or more RSV antigens and have an association rate constant or kₒₙ rate (antibody (Ab)+antigen (Ag)-Kₒₙ->Ab−Ag) of between about 10⁵ M⁻¹ s⁻¹ to about 10⁸ M⁻¹ s⁻¹ (or higher), and in certain embodiments is at least 10⁵ M⁻¹ s⁻¹, at least 2×10⁵ M⁻¹ s⁻¹, at least 4×10⁵M⁻¹ s⁻¹, at least 5×10⁵M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In another embodiment, the antibodies used in accordance with the methods of the invention immunospecifically bind to a RSV antigen and have a kₒₙ rate that is 1-fold, 1.5-fold, 2-fold, 3-fold, 4-fold or 5-fold higher than a known anti-RSV antibody. In a other embodiment, the antibodies used in accordance with the methods of the invention immunospecifically bind to a RSV F antigen and have a kₒₙ rate that is 1-fold, 2-fold, 3-fold, 4-fold, 5-fold or higher than palivizumab. A more detailed explanation of individual rate constant and affinity calculations can be found in the BIAevaluation Software Handbook (BIAcore, Inc., Piscataway, N.J.) and Kuby (1994) Immunology, 2^{nd} Ed. (W.H. Freeman & Co., New York, N.Y.).

In a specific embodiment, the antibodies used in accordance with the methods of the invention immunospecifically bind to one or more RSV antigens and have a k_{off} rate (Ab−Ag-K_{off}->Ab+Ag) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, and preferably less than 5×10⁻⁴ s⁻¹, less than 10⁻⁴ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, less than 5×10⁻¹⁰ s⁻¹, or less than 10⁻¹⁰ s⁻¹. In another embodiment, the antibodies used in accordance with the methods of the invention immunospecifically bind to a RSV antigen and have a k_{off} rate that is 1-fold, 1.5-fold, 2-fold, 3-fold, 4-fold, 5-fold, 10-fold, 20-fold, 30-fold, 40-fold, 50-fold, 60-fold, 70-fold, 80-fold, 90-fold, or 100-fold lower than a known anti-RSV antibody. In a other embodiment, the antibodies used in accordance with the methods of the invention immunospecifically bind to a RSV F antigen and have a k_{off} rate that is 1-fold, 2-fold, 3-fold, 4-fold, 5-fold, 10-fold, 15-fold, 20-fold, 30-fold, 40-fold, 50-fold, 60-fold, 70-fold, 80-fold, 90-fold, or 100-fold or lower than palivizumab.

In a specific embodiment, the antibodies used in accordance with the methods of the invention immunospecifically bind to one or more RSV antigens have a kₒₙ of between about 10⁵ M⁻¹ s⁻¹ and 10⁸ M⁻¹ s⁻¹ (or higher), and in certain embodiments is at least 10⁵ M⁻¹ s⁻¹, preferably at least 2×10⁵ M⁻¹ s⁻¹, at least 4×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹ and also have a k_{off} rate of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, and preferably less than 5×10⁻⁴ s⁻¹, less than 10⁻⁴ s⁻¹, less than 7.5×10⁻⁵ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, less than 5×10⁻¹⁰ s⁻¹, or less than 10⁻¹⁰ s⁻¹. In one embodiment, an antibody of the invention has a kₒₙ that is about 2-fold, about 3-fold, about 4-fold, or about 5-fold, or higher than palivizumab. In another embodiment, an antibody of the invention has a k_{off} that is about 2-fold, about 3-fold, about 4-fold, or about 5-fold, or lower than palivizumab.

In a specific embodiment, the antibodies used in accordance with the methods of the invention immunospecifically bind to one or more RSV antigens and have an affinity constant or Kₐ (kₒₙ/k_{off}) of from about 10² M⁻¹ to about 5×10¹⁵ M⁻¹, and in certain embodiments is at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷M⁻¹, at least 5×10⁷ M⁻¹, at least 10⁸M⁻¹, and preferably at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹²M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹.

In one embodiment, an antibody used in accordance with the methods of the invention has a dissociation constant or K_{d} (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, less than 10⁻¹⁵M, or less than 5×10⁻¹⁶ M.

In a specific embodiment, the antibodies used in accordance with the methods of the invention immunospecifically bind to a RSV antigen and have a dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 M, less than 100 pM, less than 75 pM as assessed using an described herein or known to one of skill in the art (e.g., a BIAcore assay). In another embodiment, the antibodies used in accordance with the methods of the invention immunospecifically bind to a RSV antigen and have a dissociation constant (K_{d}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using an described herein or known to one of skill in the art (e.g., a BIAcore assay or Kinexa assay). In another embodiment, the antibodies used in accordance with the methods of the invention immunospecifically bind to a RSV antigen and have a dissociation constant (K_{d}) of 500 pM, preferably 100 pM, more preferably 75 pM and most preferably 50 pM as assessed using an described herein or known to one of skill in the art (e.g., a BIAcore assay or Kinexa assay).

The present invention also provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof, said methods comprising administering to a subject a composition (for example, by pulmonary delivery or intranasal delivery) comprising one or more antibodies of the invention which immunospecifically bind to one or more RSV antigens (e.g., RSV F antigen) with higher affinity and/or higher avidity than known antibodies such as, e.g., palivizumab (e.g., antibodies or antibody fragments having an affinity of from about 2×10⁸ M⁻¹ to about 5×10¹² M⁻¹ (or higher), and preferably at least 2×10⁸M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹² M⁻¹ for one or more RSV antigens).

The IC₅₀ is the concentration of antibody that neutralizes 50% of the RSV in an in vitro microneutralization assay. In certain embodiments, the microneutralization assay is a microneutralization assay described herein or as in Johnson et al., 1999, J. Infectious Diseases 180:35-40. In specific embodiments, the antibodies used in accordance with the methods of the invention immunospecifically bind to one or more RSV antigens and have a median inhibitory concentration (IC₅₀) of less than 6 nM, less than 5 nM, less than 4 nM, less than 3 nM, less than 2 nM, less than 1.75 nM, less than 1.5 nM, less than 1.25 nM, less than 1 nM, less than 0.75 nM, less than 0.5 nM, less than 0.25 nM, less than 0.1 nM, less than 0.05 nM, less than 0.025 nM, or less than 0.01 nM, in an in vitro microneutralization assay.

Thus, methods of the invention encompass the use of modified antibodies which have increased in vivo half-lives compared to known anti-RSV antibodies as a result of, e.g., one or more modifications in amino acid residues identified to be involved in the interaction between the Fc domain of said modified antibodies and the FcRn receptor. In one embodiment, the methods of the invention encompass the use of an antibody that immunospecifically binds to a RSV antigen (e.g., RSV F antigen) with a high affinity and/or high avidity, and which comprises a modified IgG constant domain, or FcRn-binding fragment thereof (preferably, Fc domain or hinge-Fc domain), wherein the modified IgG constant domain results in increased affinity of the modified IgG constant domain for the FcRn relative to the same antibody that does not comprise a modified IgG domain or another RSV-antibody, such as the Fc domain of palivizumab. In accordance with this embodiment, the increased affinity of the Fc domain of said modified antibodies results in an in vivo half-life of said modified antibodies of from about 20 days to about 180 days (or more) and in some embodiments is at least 20 days, at least 25 days, at least 30 days, at least 35 days, at least 40 days, at least 45 days, at least 50 days, at least 60 days, at least 75 days, at least 90 days, at least 105 days, at least 120 days, at least 135 days, at least 150 days, at least 165 days, at least 180 days or longer. In another embodiment, the modified antibody comprises the VH and VL CDRs, domain or chain of MEDI-524, or an antigen-binding fragment thereof, and an Fc domain with increased affinity for the FcRn receptor relative to the Fc domain of, e.g., palivizumab.

Embodiments of the invention include, but are not limited to, the following:

1. A modified antibody that immunospecifically binds to a RSV F antigen, said modified antibody comprising three variable heavy complementarity determining regions (VH CDRs) and three variable light CDRs (VL CDRs) having an amino acid sequence of a VH CDR 1, 2 and 3 and VL CDR 1, 2 and 3 of A4B4L1FR-S28R, of A4B4-F52S, of AFFF, of P12f2, of P12f4, of P11d4, of A1e9, of A12a6, of A13c4, of A17d4, of A4B4, of A8c7, of IX-493L1 FR, of H3-3F4, of M3H9, of Y10H6, of DG, of AFFF(1), of 6H8, of L1-7E5, of L2-15B10, of A13a11, of A1h5, or of A4B4(1), as shown in Table 1, wherein said modified antibody has a modified human IgG Fc domain comprising one or more amino acid substitutions relative to a wild-type human IgG Fc domain, wherein said amino acid substitutions results in said modified antibody comprising an altered binding affinity for one or more Fc receptors as compared to a wild-type antibody without said amino acid substitutions.
2. The modified antibody of embodiment 1, wherein said modified antibody comprises a VH domain and a VL domain having an amino acid sequence of a VH domain and a VL domain of A4B4L1FR-S28R, of A4B4-F52S, of AFFF, of P12f2, of P12f4, of P11d4, of A1e9, of A12a6, of A13c4, of A17d4, of A4B4, of A8c7, of IX-493L1FR, of H3-3F4, of M3H9, of Y10H6, of DG, of AFFF(1), of 6H8, of L1-7E5, of L2-15B10, of A13a11, of A1h5, or of A4B4(1) as shown in Table 1.
3. The modified antibody of embodiment 1, wherein the modified IgG Fc domain comprises an amino acid substitution at amino acid residue 332E, as numbered by the EU index as set forth in Kabat.
4. The modified antibody of embodiment 3, wherein the modified IgG Fc domain further comprises amino acid substitutions at amino acid residues 239D and 330L, as numbered by the EU index as set forth in Kabat.
5. The modified antibody of embodiment 1, wherein the one or more amino acid substitutions is selected from the group consisting of: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat.
6. The modified antibody of embodiment 1, wherein the modified IgG Fc domain comprises an amino acid substitution at amino acid residue 331S, as numbered by the EU index as set forth in Kabat.
7. The modified antibody of embodiment 6, wherein the modified IgG Fc domain further comprises amino acid substitutions at amino acid residues 234F and 235E, as numbered by the EU index as set forth in Kabat.
8. The modified antibody of embodiment 1, wherein the one or more amino acid substitutions is selected from the group consisting of: 233P, 234V, 235A, 265A, 327G, and 330S, wherein the numbering system is that of the EU index as set forth in Kabat.
9. The modified antibody of any one of embodiments 3-7, wherein the modified IgG Fc domain further comprises additional amino acid substitutions relative to a wild-type human IgG Fc domain, wherein said additional amino acid substitutions results in an modified antibody having an extended serum half-life as compared to a wild-type antibody without said additional amino acid substitutions.
10. The modified antibody of embodiment 9, wherein said additional amino acid substitutions are at one or more of amino acid residues 251, 252, 254, 255, 256, 308, 309, 311, 312, 314, 385, 386, 387, 389, 428, 433, 434 and 436, wherein the numbering system is that of the EU index as set forth in Kabat.
11. The modified antibody of embodiment 10, wherein said additional amino acid substitutions are substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, or substitution with histidine, tyrosine, arginine or threonine at position 436, wherein the numbering system is that of the EU index as set forth in Kabat.
12. The modified antibody of embodiment 11, wherein said additional amino acid substitutions are substitutions with tyrosine at position 252, threonine at position 254 and glutamate at 256, wherein the numbering system is that of the EU index as set forth in Kabat.
13. A composition comprising the modified antibody of embodiments 1, 3, 6 or 9 in a sterile carrier.
14. A method of treating a human patient infected with RSV, the method comprising administering to said patient in need thereof a therapeutically effective amount of the composition of any one of embodiments 1-13.
15. The method of embodiment 14, wherein the therapeutically effective amount is selected from the group consisting of about 100 mg/kg, of about 50 mg/kg, of about 30 mg/kg, about 25 mg/kg, about 20 mg/kg, about 15 mg/kg, about 10 mg/kg, about 5 mg/kg, about 3 mg/kg, about 1.5 mg/kg, about 1 mg/kg, about 0.75 mg/kg, about 0.5 mg/kg, about 0.25 mg/kg, about 0.1 mg/kg, about 0.05 mg/kg, and about 0.025 mg/kg.
16. The method of embodiment 14, wherein said human patient has had a bone marrow transplant, has cystic fibrosis, has bronchopulmonary dysplasia, has congenital heart disease, has chronic obstructive pulmonary disease (COPD), has congenital immunodeficiency or has acquired immunodeficiency.
17. The method of embodiment 14, wherein said human patient is an infant, an infant born prematurely, an infant who has been hospitalized for a RSV infection, or an infant predisposed to asthma and/or reactive airway disease (RAD), and/or wheezing or a child aged 0 to 5 years.
18. The method of embodiment 14, wherein the human patient is an elderly human, or is living in a nursing home.
19. The method of embodiment 14, wherein said composition is administered to said human patient by intranasal delivery, intramuscular delivery, intradermal delivery, intraperitoneal delivery, intravenous delivery, subcutaneous delivery, oral delivery, pulmonary delivery or combinations thereof.
20. The method of embodiment 14, wherein the composition is administered to the patient five times, four times, three times, two times or one time during a RSV season.
21. The method of embodiment 14, wherein said therapeutic administration of said modified antibody inhibits or downregulates RSV replication in said human patient by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% as compared to a control in which no therapeutic administration of said modified antibody is performed, as measured by viral shedding.
22. The method of embodiment 14, wherein said therapeutic administration of said modified antibody decreases serum levels of cytokines in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.
23. The method of embodiment 14, wherein said therapeutic administration of said modified antibody decreases serum levels of chemokine release in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.
24. A method of treating a human patient infected with RSV, comprising administering a therapeutically effective amount of a fusion protein comprising a CDR having the amino acid sequence of a CDR listed in Table 1 and a heterologous amino acid sequence.
25. The method of embodiment 14, wherein said therapeutic administration of said modified antibody are administered intranasally 12 hours or 24 hours post RSV-infection to a human patient who presents with an RSV viral load of about an M.O.I of 0.1.
26. The method of embodiment 25, wherein said therapeutic administration of said modified antibody are administered intranasally 48 hours post RSV-infection to a human patient who presents with an RSV viral load of about an M.O.I of 0.01.
27. The method of embodiment 14, wherein the modified antibody is at least 80%, at least 85%, at least 90%, at least 95%, or at least 99% identical to the VH domain and VL domain amino acid sequence of AFFF, P12f2, P12f4, P11d4, A1e9, A12a6, A13c4, A17d4, A4B4, A8c7, IX-493LER, H3-3F4, M3H9, Y10H6, DG, AFFF(I), 6H8, L1-7E5, L2-15B10, A13a11, A1h5, A4B4(1), A4B4L1FR-S28R, or A4B4-F52S as shown in Table 1.
28. The method of embodiment 14, wherein said modified antibody comprises an amino acid sequence of one or more VH CDRs that are at least 80%, at least 85%, at least 90%, at least 95%, or at least 99% identical to any of the VH CDRs listed in Table 1.
29. The method of embodiment 14, wherein said modified antibody comprises an amino acid sequence of one or more VL CDRs that are at least 80%, at least 85%, at least 90%, at least 95%, or at least 99% identical to any of the VL CDRs listed in Table 1.
30. The method of any of the above embodiments, wherein said modified antibody is an Fab′2 fragment.
31. A modified antibody that immunospecifically binds to a RSV F antigen, said modified antibody comprising:
(a) a heavy chain comprising:
   (1) a heavy chain variable (VH) domain having the amino acid sequence SEQ ID NO:48,
   (2) a VH chain having the amino acid sequence SEQ ID NO:254;
   (3) a VH CDR1 having the amino acid sequence SEQ ID NO:10;
   (4) a VH CDR2 sequence having the amino acid sequence SEQ ID NO:19;
   (5) a VH CDR3 having the amino acid sequence SEQ ID NO:20;
   (6) a VH CDR1 having the amino acid sequence SEQ ID NO:10 and a VH CDR2 sequence having the amino acid sequence SEQ ID NO:19;
   (7) a VH CDR1 having the amino acid sequence SEQ ID NO:10 and a VH CDR3 having the amino acid sequence SEQ ID NO:20;
   (8) a VH CDR2 sequence having the amino acid sequence SEQ ID NO:19 and a VH CDR3 having the amino acid sequence SEQ ID NO:20; or
   (9) a VH CDR1 having the amino acid sequence SEQ ID NO:10, a VH CDR2 sequence having the amino acid sequence SEQ ID NO:19, and a VH CDR3 having the amino acid sequence SEQ ID NO:20; and/or
(b) a light chain comprising:
   (1) a light chain variable (VL) domain having the amino acid sequence SEQ ID NO:11,
   (2) a VL chain having the amino acid sequence SEQ ID NO:255;
   (3) a VL CDR1 having the amino acid sequence SEQ ID NO:39;
   (4) a VL CDR1 having the amino acid sequence SEQ ID NO:39 and a VL CDR2 sequence having the amino acid sequence SEQ ID NO:5;
   (5) a VL CDR1 having the amino acid sequence SEQ ID NO:39 and a VL CDR3 having the amino acid sequence SEQ ID NO:6; or
   (6) a VL CDR1 having the amino acid sequence SEQ ID NO:39, a VL CDR2 sequence having the amino acid sequence SEQ ID NO:5, and a VL CDR3 having the amino acid sequence SEQ ID NO:6; and
(c) wherein said modified antibody has a modified human IgG Fc domain comprising one or more amino acid substitutions relative to a wild-type human IgG Fc domain, wherein said amino acid substitutions results in an modified antibody having a modified effector function comprising an altered binding affinity for one or more FcRs as compared to a wild-type antibody without said amino acid substitutions.
   32. The modified antibody of embodiment 31, wherein the modified IgG Fc domain comprises an amino acid substitution at amino acid residue 332E, as numbered by the EU index as set forth in Kabat.
   33. The modified antibody of embodiment 32, wherein the modified IgG Fc domain further comprises amino acid substitutions at amino acid residues 239D and 330L, as numbered by the EU index as set forth in Kabat.
   34. The modified antibody of embodiment 31, wherein the one or more amino acid substitutions is selected from the group consisting of: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat.
   35. The modified antibody of embodiment 31, wherein the modified IgG Fc domain comprises an amino acid substitution at amino acid residue 331S, as numbered by the EU index as set forth in Kabat.
   36. The modified antibody of embodiment 35, wherein the modified IgG Fc domain further comprises amino acid substitutions at amino acid residues 234F and 235E, as numbered by the EU index as set forth in Kabat.
   37. The modified antibody of embodiment 31, wherein the one or more amino acid substitutions is selected from the group consisting of: 233P, 234V, 235A, 265A, 327G, and 330S, wherein the numbering system is that of the EU index as set forth in Kabat.
   38. The modified antibody of any one of embodiments 31-37, wherein the modified IgG Fc domain further comprises an additional amino acid substitutions relative to a wild-type human IgG Fc domain, wherein said additional amino acid substitutions results in an modified antibody having an extended serum half-life as compared to a wild-type antibody without said additional amino acid substitutions.
   39. The modified antibody of embodiment 38, wherein said additional amino acid substitutions are at one or more of amino acid residues 251, 252, 254, 255, 256, 308, 309, 311, 312, 314, 385, 386, 387, 389, 428, 433, 434 and 436, wherein the numbering system is that of the EU index as set forth in Kabat.
   40. The modified antibody of embodiment 39, wherein said additional amino acid substitutions are substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, or substitution with histidine, tyrosine, arginine or threonine at position 436, wherein the numbering system is that of the EU index as set forth in Kabat.
   41. The modified antibody of embodiment 40, wherein said additional amino acid substitutions are substitutions with tyrosine at position 252, threonine at position 254 and glutamate at 256, wherein the numbering system is that of the EU index as set forth in Kabat.
   42. The modified antibody of embodiment 41, wherein the in vivo half-life of the modified antibody is extended by about two-fold, about three-fold, about four-fold, about five-fold, about six-fold, about seven-fold, about eight-fold, about nine-fold, or about ten fold as compared to the same antibody comprising an IgG Fc domain without a tyrosine at position 252, a threonine at position 254 is a threonine, and a glutamic acid at position 256.
   43. The modified antibody of embodiment 1, 8, 31 or 38, wherein the antibody has an association rate (kₒₙ) of at least about 2×10⁵ M⁻¹ s⁻¹.
   44. The modified antibody of embodiment 43, wherein the kₒₙ is at least about 7.5×10⁵ s⁻¹.
   45. The modified antibody of embodiment 1, 8, 31 or 38, wherein the antibody has a dissociation rate (k_{off}) of less than about 5×10⁻⁴ s⁻¹.
   46. The modified antibody of embodiment 1, 8, 31 or 38, wherein the antibody has a dissociation constant (k_{d}) of less than about 1000 pM.
   47. The modified antibody of embodiment 1, 8, 31 or 38, wherein the antibody has an association constant of (Kₐ) of at least about 10⁹ M⁻¹.
   48. A composition comprising the modified antibody of any one of embodiments 31-47 in a sterile carrier.
   49. A method of treating a human patient infected with RSV, the method comprising administering to said patient in need thereof a therapeutically effective amount of the composition of any one of embodiments 31-48.
   50. The method of embodiment 49, wherein the therapeutically effective amount is selected from the group consisting of about 100 mg/kg, about 50 mg/kg, about 30 mg/kg, about 25 mg/kg, about 20 mg/kg, about 15 mg/kg, about 10 mg/kg, about 5 mg/kg, about 3 mg/kg, about 1.5 mg/kg, about 1 mg/kg, about 0.75 mg/kg, about 0.5 mg/kg, about 0.25 mg/kg, about 0.1 mg/kg, about 0.05 mg/kg, and about 0.025 mg/kg.
   51. The method of embodiment 49, wherein said human patient has had a bone marrow transplant, has cystic fibrosis, has bronchopulmonary dysplasia, has congenital heart disease, has chronic obstructive pulmonary disease (COPD), has congenital immunodeficiency or has acquired immunodeficiency.
   52. The method of embodiment 49, wherein said human patient is an infant, an infant born prematurely, an infant who has been hospitalized for a RSV infection, or an infant predisposed to asthma and/or reactive airway disease (RAD), and/or wheezing child aged 0 to 5 years.
   53. The method of embodiment 49, wherein the human patient is an elderly human, or is living in a nursing home.
   54. The method of embodiment 49, wherein said composition is administered to said human patient by intranasal delivery, intramuscular delivery, intradermal delivery, intraperitoneal delivery, intravenous delivery, subcutaneous delivery, oral delivery, pulmonary delivery or combinations thereof.
   55. The method of embodiment 49, wherein the composition is administered to the patient five times, four times, three times, two times or one time during a RSV season.
   56. The method of embodiment 49, wherein said therapeutic administration of said modified antibody inhibits or downregulates RSV replication in said human patient by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% as compared to a control in which no therapeutic administration of said modified antibody is performed, as measured by viral shedding.
   57. The method of embodiment 49, wherein said therapeutic administration of said modified antibody decreases serum levels of cytokines in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.
   58. The method of embodiment 49, wherein said therapeutic administration of said modified antibody decreases serum levels of chemokine release in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.
   59. A method of treating a human patient infected with RSV, comprising administering a therapeutically effective amount of a fusion protein comprising a CDR having the amino acid sequence of a CDR listed in Table 1 and a heterologous amino acid sequence.
   60. The method of embodiment 49, wherein said therapeutic administration of said modified antibody are administered intranasally 12 hours or 24 hours post RSV-infection to a human patient who presents with an RSV viral load of about an M.O.I of 0.1.
   61. The method of embodiment 49, wherein said therapeutic administration of said modified antibody are administered intranasally 48 hours post RSV-infection to a human patient who presents with an RSV viral load of about an M.O.I of 0.01.
   62. A method of treating a human patient infected with RSV, the method comprising administering to said patient in need thereof a therapeutically effective amount of a F(ab)′ fragment comprising three variable heavy complementarity determining regions (VH CDRs) and three variable light CDRs (VL CDRs) having an amino acid sequence of A4B4L1FR-S28R, of A4B4-F52S, of AFFF, of P12f2, of P12f4, of P11d4, of A1e9, of A12a6, of A13c4, of A17d4, of A4B4, of A8c7, of IX-493L1FR, of H3-3F4, of M3H9, of Y10H6, of DG, of AFFF(I), of 6H8, of L1-7E5, of L2-15B10, of A13a11, of A1 h5, or of A4B4(1), as shown in Table 1, wherein said administration is pulmonary and is during the RSV season.
   63. The method of embodiment 62, wherein said human patient is an adult or an elderly patient.
   64. The method of embodiment 63, wherein hospitalization of said patient due to COPD in said patient is mitigated or avoided, as compared to a similar patient who did not receive a therapeutically effective amount of said F(ab)′ fragment or placebo.
   65. The method of embodiments 14 or 49, wherein a hospitalization period of said human patient is reduced by at least 60%, at least 75%, at least 85%, at least 95%, or at least 99% as compared to placebo or a human who did not receive a therapeutic administration of said antibodies.

5.1 Antibodies

It should be recognized that antibodies that immunospecifically bind to a RSV antigen are known in the art. For example, palivizumab is a humanized monoclonal antibody presently used for the prevention of RSV infection in pediatric patients. The present invention provides methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof by administering to a subject an effective amount of a modified anti-RSV antibody of the invention as described in Table 1 or an antigen-binding fragment thereof.

The present invention also provides modified antibodies and methods for treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof by administering to a subject an effective amount of an anti-RSV antibody of the invention, wherein the antibody comprises a modified IgG constant domain, or FcRn-binding fragment thereof (preferably, Fc domain or hinge-Fc domain).

In one embodiment, the modified antibody has one or more amino acid modifications. The one or more amino acid modifications may be substitutions. In one embodiment, the one or more amino acid substitutions are: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat. Such Fc domain amino acid substitutions encompass an increase in ADCC (3M) if compared to the same antibody without said amino acid substitutions. A specific embodiment for 3M includes, but is not limited to, 239D, 330L, and 332E.

In another embodiment, the one or more amino acid substitutions is selected from the group consisting of: 233P, 234F, 234V, 235A, 235E, 265A, 327G, 330S, and 331S, wherein the numbering system is that of the EU index as set forth in Kabat. Such Fc domain amino acid substitutions encompass a decrease in ADCC (TM) if compared to the same antibody without said amino acid substitutions. A specific embodiment for TM includes, but is not limited to, 234F, 235E, and 331S.

In another embodiment, the one or more amino acid modifications are, in addition to those described for 3M and TM, in combination with those at positions 251-256, 285-290, 308-314, 385-389, and 428-436, with numbering according to the EU Index as in Kabat. Such Fc domain combination amino acid substitutions encompass a modified antibody having either an increase in ADCC (3M) with an increase in in vivo half life, or a modified antibody having a decrease in ADCC (TM) with an increase in in vivo half life, if both are compared to the same antibody without said amino acid substitutions. In certain embodiments, an IgG constant domain comprises a 239D, 330L, 332E, 252Y, 254T, and 256E. In other embodiments, an IgG constant domain comprises a 234F, 235E, 331S, 252Y, 254T, and 256E.

The present invention provides antibodies (modified) that immunospecifically bind to one or more RSV antigens. Preferably, the antibodies of the invention immunospecifically bind to one or more RSV antigens regardless of the strain of RSV. The present invention also provides antibodies that differentially or preferentially bind to RSV antigens from one strain of RSV versus another RSV strain. In a specific embodiment, the antibodies of the invention immunospecifically bind to the RSV F glycoprotein, G glycoprotein or SH protein. In another embodiment, the antibodies present invention immunospecifically bind to the RSV F glycoprotein. In another embodiment, the antibodies of the present invention bind to the A, B, or C antigenic sites of the RSV F glycoprotein.

Antibodies of the invention include, but are not limited to, monoclonal antibodies, multispecific antibodies, human antibodies, humanized antibodies, chimeric antibodies, single domain antibodies, camelised antibodies, single chain Fvs (scFv) single chain antibodies, Fab fragments, F(ab′) fragments, disulfide-linked Fvs (sdFv) intrabodies, and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a RSV antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody (modified) of the invention is an IgG antibody, preferably an IgG1. In another specific embodiment, an antibody of the invention is not an IgA antibody.

The antibodies of the invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies of the present invention may be monospecific, bispecific, trispecific or of greater multispecificity. Multispecific antibodies may be specific for different epitopes of a RSV polypeptide or may be specific for both a RSV polypeptide as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that exhibit a high potency in an assay described herein. High potency antibodies can be produced by methods disclosed in copending U.S. patent application Ser. Nos. 60/168,426, 60/186,252, U.S. Publication No. 2002/0098189, and U.S. Pat. No. 6,656,467 (which are incorporated herein by reference in their entirety) and methods described herein. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay.

In a specific embodiment, an antibody of the invention has approximately 20-fold, 25-fold, 30-fold, 35-fold, 40-fold, 45-fold, 50-fold, 55-fold, 60-fold, 65-fold, 70-fold, 75-fold, 80-fold, 90-fold, 100-fold or higher affinity for a RSV antigen (e.g., RSV F antigen) than palivizumab or an antibody-binding fragment thereof as assessed by an assay known in the art or described herein (e.g., a BIAcore assay). In another embodiment, an antibody of the invention has an approximately 1-fold, 1.5-fold, 2-fold, 3-fold, 4-fold, 5-fold, or a higher Kₐ than palivizumab or an antigen-binding fragment thereof as assessed by an assay known in the art or described herein. In another embodiment, an antibody of the invention has an approximately 1-fold, 2-fold, 3-fold, 4-fold, 5-fold, 6-fold, 7-fold, 8-fold, 9-fold, 10-fold, 11-fold 12-fold, 13-fold, 14-fold, 15-fold, 16-fold, 17-fold, 18-fold, 19-fold, or 20-fold or more potent than palivizumab or an antigen-binding fragment thereof in an in vitro microneutralization assay. In certain embodiments, the microneutralization assay is a microneutralization assay described herein or as in Johnson et al., 1999, J. Infectious Diseases 180:35-40. The amino acid sequence of palivizumab is disclosed, e.g., in Johnson et al., 1997, J. Infectious Disease 176:1215-1224 which is incorporated herein by reference in its entirety. In some embodiments, an antibody of the invention is an antibody comprising a VH domain of SEQ ID NO:7 (or VH chain of SEQ ID NO:208) and/or a VL domain of SEQ ID NO:8 (or VL chain of SEQ ID NO:209) comprising a modified IgG (e.g., IgG1) constant domain, or FcRn binding fragment thereof (e.g., the Fc domain or hinge-Fc domain), described herein. In some embodiments, an antibody of the invention is an antibody comprising a VH domain of SEQ ID NO:7 (or VH chain of SEQ ID NO:208) and/or a VL domain of SEQ ID NO:8 (or VL chain of SEQ ID NO:209) comprising a modified IgG (e.g., IgG1) constant domain, or FcRn binding fragment thereof (e.g., the Fc domain or hinge-Fc domain), described herein. In other embodiments, a modified antibody of the invention is a modified palivizumab antibody or a modified antibody comprising a VH domain of SEQ ID NO:7 (or VH chain of SEQ ID NO:208) and/or a VL domain of SEQ ID NO:8 (or VL chain of SEQ ID NO:209) comprising a modified IgG (e.g., IgG1) constant domain, or FcRn binding fragment thereof (e.g., the Fc domain or hinge-Fc domain), described herein.

In another embodiment, the present invention provides for modified antibodies that immunospecifically bind to one or more RSV antigens, said antibodies comprising one, two, three, or more CDRs having the amino acid sequence of one, two, three, or more CDRs of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, and/or A17h4 (see Table 1) comprising a modified IgG (e.g., IgG1) constant domain, or FcRn binding fragment thereof (e.g., the Fc domain or hinge-Fc domain), described herein. In a other embodiment, an antibody of the invention immunospecifically binds to a RSV antigen, and said antibody comprises one, two, three, or more CDRs having the amino acid sequence of one, two, three, or more CDRs of MEDI-524 comprising a modified IgG (e.g., IgG1) constant domain, or FcRn binding fragment thereof (e.g., the Fc domain or hinge-Fc domain), described herein. In yet another embodiment, the present invention provides for one or more antibodies that immunospecifically bind to one or more RSV F antigens, said antibodies comprising a combination of VH CDRs and/or VL CDRs having the amino acid sequence of VH CDRs and/or VL CDRs of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, and/or A17h4, as shown in Table 1, comprising a modified IgG (e.g., IgG1) constant domain, or FcRn binding fragment thereof (e.g., the Fc domain or hinge-Fc domain), described herein. In a other embodiment, an antibody of the invention immunospecifically binds to a RSV F antigen and said antibody comprises a combination of VH CDRs and/or VL CDRs having the amino acid sequence of the VH CDRs and/or VL CDRs of MEDI-524 (e.g., A4B4L1FR-S28R as shown in Table 1), comprising a modified IgG (e.g., IgG1) constant domain, or FcRn binding fragment thereof (e.g., the Fc domain or hinge-Fc domain), described herein.

TABLE 1
Antibodies & Fragments Thereof

| Antibody | VH | VH | | | | VL | VL | | | |
|---|---|---|---|---|---|---|---|---|---|---|
| Name | Chain | Domain | VH CDR1 | VH CDR2 | VH CDR3 | Chain | Domain | VL CDR1 | VL CDR2 | VL CDR3 |
| **palivizum | SEQ ID | SEQ ID | TSGMSVG | DIWWDDKKDYN | SMITNWYFDV | SEQ ID | SEQ ID | KCQLSVGYMH | DTSKLAS | FQGSGYPFT |
| ab | NO: 208 | NO: 7 | (SEQ ID | PSLKS | (SEQ ID | NO: 209 | NO: 8 | (SEQ ID | (SEQ ID | (SEQ ID |
| | | | NO: 1) | (SEQ ID | NO: 3) | | | NO: 4) | NO: 5) | NO: 6) |
| | | | | NO: 2) | | | | | | |
| ***AFFF | SEQ ID | SEQ ID | TAGMSVG | DIWWDDKKDYN | SMITNFYFDV | SEQ ID | SEQ ID | SASSSVGYMH | DTFKLAS | FQFSGYPFT |
| | NO: 210 | NO: 9 | (SEQ ID | PSLKS | (SEQ ID | NO: 211 | NO: 13 | (SEQ ID | (SEQ ID | (SEQ ID |
| | | | NO: 10) | (SEQ ID | NO: 12) | | | NO: 14) | NO: 15) | NO: 16) |
| | | | | NO: 2) | | | | | | |
| ***P12f2 | SEQ ID | SEQ ID | TPGMSVG | DIWWDDKKHYN | DMIFNFYFDV | SEQ ID | SEQ ID | SLSSRVGYMH | DTFYLSS | FQGSGYPFT |
| | NO: 212 | NO: 17 | (SEQ ID | PSLKD | (SEQ ID | NO: 213 | NO: 21 | (SEQ ID | (SEQ ID | (SEQ ID |
| | | | NO: 18) | (SEQ ID | NO: 20) | | | NO: 22) | NO: 23) | NO: 6) |
| | | | | NO: 19) | | | | | | |
| ***P12f4 | SEQ ID | SEQ ID | TPGMSVG | DIWWDGKKHYN | DMIFNFYFDV | SEQ ID | SEQ ID | SLSSRVGYMH | DTRGLPS | FQGSGYPFT |
| | NO: 214 | NO: 24 | (SEQ ID | PSLKD | (SEQ ID | NO: 215 | NO: 26 | (SEQ ID | (SEQ ID | (SEQ ID |
| | | | NO: 18) | (SEQ ID | NO: 20) | | | NO: 22) | NO: 27) | NO: 6) |
| | | | | NO: 25) | | | | | | |
| ***P11d4 | SEQ ID | SEQ ID | TPGMSVG | DIWWDGKKHYN | DMIFNWYFDV | SEQ ID | SEQ ID | SPSSRVGYMH | DTMRLAS | FQGSGYPFT |
| | NO: 216 | NO: 28 | (SEQ ID | PSLKD | (SEQ ID | NO: 217 | NO: 30 | (SEQ ID | (SEQ ID | (SEQ ID |
| | | | NO: 18) | (SEQ ID | NO: 29) | | | NO: 31) | NO: 32) | NO: 6) |
| | | | | NO: 25) | | | | | | |
| ***A1e9 | SEQ ID | SEQ ID | TAGMSVG | DIWWDGKKHYN | DMIFNWYFDV | SEQ ID | SEQ ID | SLSSRVGYMH | DTFKLSS | FQGSGYPFT |
| | NO: 218 | NO: 33 | (SEQ ID | PSLKD | (SEQ ID | NO: 219 | NO: 34 | (SEQ ID | (SEQ ID | (SEQ ID |
| | | | NO: 10) | (SEQ ID | NO: 29) | | | NO: 22) | NO: 35) | NO: 6) |
| | | | | NO: 25) | | | | | | |
| ***A12a6 | SEQ ID | SEQ ID | TAGMSVG | DIWWDGKKDYN | DMIFNFYFDV | SEQ ID | SEQ ID | SASSRVGYMH | DTFKLSS | FQGSGYPFT |
| | NO: 220 | NO: 36 | (SEQ ID | PSLKD | (SEQ ID | NO: 221 | NO: 38 | (SEQ ID | (SEQ ID | (SEQ ID |
| | | | NO: 10) | (SEQ ID | NO: 20) | | | NO: 39) | NO: 35) | NO: 6) |
| | | | | NO: 37) | | | | | | |
| ***A13c4 | SEQ ID | SEQ ID | TAGMSVG | DIWWDGKKSYN | DMIFNFYFDV | SEQ ID | SEQ ID | SLSSRVGYMH | DTMYQSS | FQGSGYPFT |
| | NO: 222 | NO: 40 | (SEQ ID | PSLKD | (SEQ ID | NO: 223 | NO: 42 | (SEQ ID | (SEQ ID | (SEQ ID |
| | | | NO: 10) | (SEQ ID | NO: 20) | | | NO: 22) | NO: 43) | NO: 6) |
| | | | | NO: 41) | | | | | | |
| ***A17d4 | SEQ ID | SEQ ID | TAGMSVG | DIWWDDKKSYN | DMIFNFYFDV | SEQ ID | SEQ ID | LPSSRVGYMH | DTMYQSS | FQGSGYPFT |
| | NO: 224 | NO: 44 | (SEQ ID | PSLKD | (SEQ ID | NO: 225 | NO: 46 | (SEQ ID | (SEQ ID | (SEQ ID |
| | | | NO: 10) | (SEQ ID | NO: 20) | | | NO: 47) | NO: 43) | NO: 6) |
| | | | | NO: 45) | | | | | | |
| ***A4B4 | SEQ ID | SEQ ID | TAGMSVG | DIWWDDKKHYN | DMIFNFYFDV | SEQ ID | SEQ ID | SASSRVGYMH | DTFFLDS | FQGSGYPFT |
| | NO: 226 | NO: 48 | (SEQ ID | PSLKD | (SEQ ID | NO: 227 | NO: 49 | (SEQ ID | (SEQ ID | (SEQ ID |
| | | | NO: 10) | (SEQ ID | NO: 20) | | | NO: 39) | NO: 50) | NO: 6) |
| | | | | NO: 19) | | | | | | |
| ****A8c7 | SEQ ID | SEQ ID | TAGMSVG | DIWWDDKKSYN | DMIFNWYFDV | SEQ ID | SEQ ID | SPSSRVGYMH | DTRYQSS | FQGSGYPFT |
| | NO: 228 | NO: 51 | (SEQ ID | PSLKD | (SEQ ID | NO: 229 | NO: 52 | (SEQ ID | (SEQ ID | (SEQ ID |
| | | | NO: 10) | (SEQ ID | NO: 29) | | | NO: 31) | NO: 53) | NO: 6) |
| | | | | NO: 45) | | | | | | |
| *1X-493L1FR | SEQ ID | SEQ ID | TSGMSVG | DIWWDDKKDYN | SMITNWYFDV | SEQ ID | SEQ ID | SASSSVGYMH | DTSKLAS | FQGSGYPFT |
| | NO: 230 | NO: 343 | (SEQ ID | PSLKS | (SEQ ID | NO: 231 | NO: 54 | (SEQ ID | (SEQ ID | (SEQ ID |
| | | | NO: 1) | (SEQ ID | NO: 3) | | | NO: 14) | NO: 5) | NO: 6) |
| | | | | NO: 2) | | | | | | |
| *H3-3F4 | SEQ ID | SEQ ID | TAGMSVG | DIWWDDKKDYN | DMIFNWYFDV | SEQ ID | SEQ ID | SASSSVGYMH | DTFKLAS | FQGSGYPFT |
| | NO: 232 | NO: 55 | (SEQ ID | PSLKS | (SEQ ID | NO: 233 | NO: 56 | (SEQ ID | (SEQ ID | (SEQ ID |
| | | | NO: 10) | (SEQ ID | NO: 29) | | | NO: 14) | NO: 15) | NO: 6) |
| | | | | NO: 2) | | | | | | |
| *M3H9 | SEQ ID | SEQ ID | TAGMSVG | DIWWDDKKDYN | DMIFNWYFDV | SEQ ID | SEQ ID | SASSSVGYMH | DTYKQTS | FQGSGYPFT |
| | NO: 234 | NO: 55 | (SEQ ID | PSLKS | (SEQ ID | NO: 235 | NO: 70 | (SEQ ID | (SEQ ID | (SEQ ID |
| | | | NO: 10) | (SEQ ID | NO: 29) | | | NO: 14) | NO: 57) | NO: 6) |
| | | | | NO: 2) | | | | | | |
| *Y10H6 | SEQ ID | SEQ ID | TAGMSVG | DIWWDDKKDYN | DMIFNWYFDV | SEQ ID | SEQ ID | SASSSVGYMH | DTRYLSS | FQGSGYPFT |
| | NO: 236 | NO: 55 | (SEQ ID | PSLKS | (SEQ ID | NO: 237 | NO: 58 | (SEQ ID | (SEQ ID | (SEQ ID |
| | | | NO: 10) | (SEQ ID | NO: 29) | | | NO: 14) | NO: 59) | NO: 6) |
| | | | | NO: 2) | | | | | | |
| *DG (aka | SEQ ID | SEQ ID | TAGMSVG | DIWWDDKKDYN | DMITNFYFDV | SEQ ID | SEQ ID | SASSSVGYMH | DTFKLAS | FQGSGYPFT |
| D95/G93) | NO: 238 | NO: 78 | (SEQ ID | PSLKS | (SEQ ID | NO: 239 | NO: 56 | (SEQ ID | (SEQ ID | (SEQ ID |
| | | | NO: 10) | (SEQ ID | NO: 79) | | | NO: 14) | NO: 15) | NO: 6) |
| | | | | NO: 2) | | | | | | |
| AFFF(1) | SEQ ID | SEQ ID | TAGMSVG | DIWWDDKKDYN | SMITNFYFDV | SEQ ID | SEQ ID | SASSSVGYMH | DTFKLAS | FQGSFYPFT |
| | NO: 240 | NO: 9 | (SEQ ID | PSLKS | (SEQ ID | NO: 241 | NO: 60 | (SEQ ID | (SEQ ID | (SEQ ID |
| | | | NO: 10) | (SEQ ID | NO: 12) | | | NO: 14) | NO: 15) | NO: 61) |
| | | | | NO: 2) | | | | | | |
| *6H8 | SEQ ID | SEQ ID | TAGMSVG | DIWWDDKKDYN | DMITNFYFDV | SEQ ID | SEQ ID | SASSSVGYMH | DTFKLTS | FQGSGYPFT |
| | NO: 242 | NO: 78 | (SEQ ID | PSLKS | (SEQ ID | NO: 243 | NO: 62 | (SEQ ID | (SEQ ID | (SEQ ID |
| | | | NO: 10) | (SEQ ID | NO: 79) | | | NO: 14) | NO: 63) | NO: 6) |
| | | | | NO: 2) | | | | | | |
| *L1-7E5 | SEQ ID | SEQ ID | TAGMSVG | DIWWDDKKDYN | DMITNFYFDV | SEQ ID | SEQ ID | SASSRVGYMH | DTFKLAS | FQGSGYPFT |
| | NO: 244 | NO: 78 | (SEQ ID | PSLKS | (SEQ ID | NO: 245 | NO: 64 | (SEQ ID | (SEQ ID | (SEQ ID |
| | | | NO: 10) | (SEQ ID | NO: 79) | | | NO: 39) | NO: 15) | NO: 6) |
| | | | | NO: 2) | | | | | | |
| *L2-15B10 | SEQ ID | SEQ ID | TAGMSVG | DIWWDDKKDYN | DMITNFYFDV | SEQ ID | SEQ ID | SASSSVGYMH | DTFRLAS | FQGSGYPFT |
| | NO: 246 | NO: 78 | (SEQ ID | PSLKS | (SEQ ID | NO: 247 | NO: 65 | (SEQ ID | (SEQ ID | (SEQ ID |
| | | | NO: 10) | (SEQ ID | NO: 79) | | | NO: 14) | NO: 66) | NO: 6) |
| | | | | NO: 2) | | | | | | |
| *A13a11 | SEQ ID | SEQ ID | TAGMSVG | DIWWDDKKHYN | DMIFNWYFDV | SEQ ID | SEQ ID | SPSSRVGYMH | DTYRHSS | FQGSGYPFT |
| | NO: 248 | NO: 67 | (SEQ ID | PSLKD | (SEQ ID | NO: 249 | NO: 68 | (SEQ ID | (SEQ ID | (SEQ ID |
| | | | NO: 10) | (SEQ ID | NO: 29) | | | NO: 31) | NO: 69) | NO: 6) |
| | | | | NO: 19) | | | | | | |
| *A1h5 | SEQ ID | SEQ ID | TAGMSVG | DIWWDGKKHYN | DMIFNWYFDV | SEQ ID | SEQ ID | SLSSSVGYMH | DTFFHRS | FQGSGYPFT |
| | NO: 250 | NO: 33 | (SEQ ID | PSLKD | (SEQ ID | NO: 251 | NO: 71 | (SEQ ID | (SEQ ID | (SEQ ID |
| | | | NO: 10) | (SEQ ID | NO: 29) | | | NO: 72) | NO: 73) | NO: 6) |
| | | | | NO: 25) | | | | | | |
| A4B4(1) | SEQ ID | SEQ ID | TAGMSVG | DIWWDDKKHYN | DMIFNFYFDV | SEQ ID | SEQ ID | SASSRVGYMH | DTLLLDS | FQGSGYPFT |
| | NO: 252 | NO: 48 | (SEQ ID | PSLKD | (SEQ ID | NO: 253 | NO: 74 | (SEQ ID | (SEQ ID | (SEQ ID |
| | | | NO: 10) | (SEQ ID | NO: 20) | | | NO: 39) | NO: 75) | NO: 6) |
| | | | | NO: 19) | | | | | | |
| ***A4B4L1F | SEQ ID | SEQ ID | TAGMSVG | DIWWDDKKHYN | DMIFNFYFDV | SEQ ID | SEQ ID | SASSRVGYMH | DTSKLAS | FQGSGYPFT |
| 4-S28R (aka | NO: 254 | NO: 48 | (SEQ ID | PSLKD | (SEQ ID | NO: 255 | NO: 11 | (SEQ ID | (SEQ ID | (SEQ ID |
| MEDI-524) | | | NO: 10) | (SEQ ID | NO: 20) | | | NO: 39) | NO: 5) | NO: 6) |
| | | | | NO: 19) | | | | | | |
| ***A4B4- | SEQ ID | SEQ ID | TAGMSVG | DIWWDDKKHYN | DMIFNFYFDV | SEQ ID | SEQ ID | SASSRVGYMH | DTSFLDS | FQGSGYPFT |
| F52S | NO: 256 | NO: 48 | (SEQ ID | PSLKD | (SEQ ID | NO: 257 | NO: 76 | (SEQ ID | (SEQ ID | (SEQ ID |
| | | | NO: 10) | (SEQ ID | NO: 20) | | | NO: 39) | NO: 77) | NO: 6) |
| | | | | NO: 19) | | | | | | |
| ***A17d4(1) | SEQ ID | SEQ ID | TAGMSVG | DIWWDGKKSYN | DMIFNFYFDV | SEQ ID | SEQ ID | LPSSRVGYMH | DTMYQSS | FQGSGYPFT |
| | NO: 222 | NO: 40 | (SEQ ID | (PSLKD | (SEQ ID | NO: 225 | NO: 46 | (SEQ ID | (SEQ ID | (SEQ ID |
| | | | NO: 10) | (SEQ ID | NO: 20) | | | NO: 47) | NO: 43) | NO: 6) |
| | | | | NO: 41) | | | | | | |
| ***A3e2 | SEQ ID | SEQ ID | TAGMSVG | DIWWGDKGHYN | DMIFNWYFDV | SEQ ID | SEQ ID | SASSSVGYMH | DTFYLHS | FQGSGYPFT |
| | NO: 303 | NO: 304 | (SEQ ID | PSLKD | (SEQ ID | NO: 306 | NO: 307 | (SEQ ID | (SEQ ID | (SEQ ID |
| | | | NO: 10) | (SEQ ID | NO: 29) | | | NO: 14) | NO: 308) | NO: 6) |
| | | | | NO: 305) | | | | | | |
| ***A14a4 | SEQ ID | SEQ ID | TAGMSVG | DIWWDDKKSYN | DMITNWYFDV | SEQ ID | SEQ ID | LLSSRVGYNH | DTYYQTS | FQGSGYPFT |
| | NO: 309 | NO: 310 | (SEQ ID | PSLKD | (SEQ ID | NO: 312 | NO: 313 | (SEQ ID | (SEQ ID | (SEQ ID |
| | | | NO: 10) | (SEQ ID | NO: 311) | | | NO: 314) | NO: 315) | NO: 6) |
| | | | | NO: 45 | | | | | | |
| ***A16b4 | SEQ ID | SEQ ID | TAGMSVG | DIWWDDKKHYN | DMIFNWYFDV | SEQ ID | SEQ ID | LLSSRVGYMH | DTMYQAS | FQGSGYPFT |
| | NO: 316 | NO: 317 | (SEQ ID | PSLKD | (SEQ ID | NO: 318 | NO: 319 | (SEQ ID | (SEQ ID | (SEQ ID |
| | | | NO: 10) | (SEQ ID | NO: 29) | | | NO: 320) | NO: 321) | NO: 6) |
| | | | | NO: 19) | | | | | | |
| ***A17b5 | SEQ ID | SEQ ID | TAGMSVG | DIWWDDKKHYN | DMIFNWYFDV | SEQ ID | SEQ ID | SLSSRVGYMH | DTYYLPS | FQGSGYPFT |
| | NO: 322 | NO: 323 | (SEQ ID | PSLKD | (SEQ ID | NO: 324 | NO: 325 | (SEQ ID | (SEQ ID | (SEQ ID |
| | | | NO: 10) | (SEQ ID | NO: 29) | | | NO: 22) | NO: 326) | NO: 6) |
| | | | | NO: 19) | | | | | | |
| ***A17f5 | SEQ ID | SEQ ID | TAGMSVG | DIWWDDKKDYN | DMIFNWYFDV | SEQ ID | SEQ ID | SLSSRVGYMH | DTFRHTS | FQGSGYPFT |
| | NO: 327 | NO: 328 | (SEQ ID | PSLKD | (SEQ ID | NO: 330 | NO: 331 | (SEQ ID | (SEQ ID | (SEQ ID |
| | | | NO: 10) | (SEQ ID | NO: 29) | | | NO: 22) | NO: 332) | NO: 6) |
| | | | | NO: 329) | | | | | | |
| ***A17h4 | SEQ ID | SEQ ID | TAGMSVG | DIWWDGKKHYN | DMIFNWYFDV | SEQ ID | SEQ ID | SPSSSVGYMH | DTYYLAS | FQGSGYPFT |
| | NO: 218 | NO: 33 | (SEQ ID | PSLKD | (SEQ ID | NO: 333 | NO: 334 | (SEQ ID | (SEQ ID | (SEQ ID |
| | | | NO: 10) | (SEQ ID | NO: 29) | | | NO: 335) | NO: 336) | NO: 6) |
| | | | | NO: 25) | | | | | | |

In one embodiment, Fc modified antibodies of the invention comprise a VH CDR1 having the amino acid sequence of SEQ ID NO:1, SEQ ID NO:10 or SEQ ID NO:18. In another embodiment, Fc modified antibodies of the invention comprise a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329. In another embodiment, Fc modified antibodies of the invention comprise a VH CDR3 having the amino acid sequence of SEQ ID NO:3, SEQ ID NO:12, SEQ ID NO:20, SEQ ID NO:29, SEQ ID NO:79, or SEQ ID NO:311. In another embodiment, Fc modified antibodies of the invention comprise a VH CDR1 having the amino acid sequence of SEQ ID NO:1, SEQ ID NO:10 or SEQ ID NO:18, a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VH CDR3 having the amino acid sequence of SEQ ID NO:3, SEQ ID NO:12, SEQ ID NO:20, SEQ ID NO:29, SEQ ID NO:79, or SEQ ID NO:311. In a other embodiment, Fc modified antibodies of the invention comprise a VH CDR1 having the amino acid sequence of SEQ ID NO:10, a VH CDR2 having the amino acid sequence of SEQ ID NO:19, and a VH CDR3 having the amino acid sequence of SEQ ID NO:20. In accordance with these embodiments, the antibodies immunospecifically bind to a RSV F antigen.

In one embodiment, the amino acid sequence of the VH domain of an antibody of the invention is:

(SEQ ID NO: 48)
Q V T L R E S G P A L V K P T
Q T L T L T C T F S G F S L S
T A G M S V G W I R Q P P G K
A L E W L A D I W W D D K K H
Y N P S L K D R L T I S K D T
S K N Q V V L K V T N M D P A
D T A T Y Y C A R D M I F N F
Y F D V W G Q* G T T V T V S S,

wherein the three underlined regions indicate the VH CDR1, CDR2, and CDR3 regions, respectively; the four non-underlined regions correlate with the VH FR1, FR2, FR3, FR4, respectively; and the asterisk indicates the position of an A→Q mutation in VH FR4 as compared to the VH FR4 of palivizumab (SEQ ID NO:7). This VH domain (SEQ ID NO:48) is identical to that of the MEDI-524 antibody described elsewhere herein. In some embodiments, this VH FR can be used in combination with any of the VH CDRs identified in Table 1. In one embodiment, the MEDI-524 antibody comprises the VH domain (SEQ ID NO:48) and the C-gamma-1 (nG1m) constant domain described in Johnson et al. (1997), J. Infect. Dis. 176, 1215-1224 comprising a modified IgG (e.g., IgG1) constant domain, or FcRn binding fragment thereof (e.g., the Fc domain or hinge-Fc domain), described herein. In one embodiment, an Fc modified antibody of the invention comprises a VH chain having the amino acid sequence of SEQ ID NO:208 and/or a VH domain having the amino acid sequence of SEQ ID NO:7. In another embodiment, an Fc modified antibody of the invention comprises a VH chain having the amino acid sequence SEQ ID NO:254. In another embodiment, a modified antibody of the invention comprises a VH domain having the amino acid sequence SEQ ID NO:48.

In one embodiment of the present invention, the Fc modified antibodies comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335. In another embodiment, Fc modified antibodies of the invention comprise a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, Fc modified antibodies of the invention comprise a VL CDR3 having the amino acid sequence of SEQ ID NO:6, SEQ ID NO:16 or SEQ ID NO:61. In another embodiment, Fc modified antibodies of the invention comprise a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:72, SEQ ID NO:314, SEQ ID NO:320, or SEQ ID NO:335, a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336, and a VL CDR3 having the amino acid sequence of SEQ ID NO:6, SEQ ID NO:16 or SEQ ID NO:61. In a other embodiment, Fc modified antibodies of the invention comprise a VL CDR1 having the amino acid sequence of SEQ ID NO:39, a VLCDR2 having the amino acid sequence of SEQ ID NO:5, and a VLCDR3 having the amino acid sequence of SEQ ID NO:6. In a specific embodiment, the antibodies have a high affinity for RSV antigen (e.g., RSV F antigen).

In one embodiment the amino acid sequence of the VL domain of an antibody of the invention is:

(SEQ ID NO: 11)
D I Q M T Q S P S T L S A S V
G D R V T I T C S A S S R V G
Y M H W Y Q Q K P G K A P K L
L I Y D T S K L A S G V P S R
F S G S G S G T E F T L T I S
S L Q P D D F A T Y Y C F Q G
S G Y P F T F G G G T K V* E I
K,

wherein the three underlined regions indicate the VL CDR1, CDR2, and CDR3 regions, respectively; the four non-underlined regions correlate with the VL FR1, FR2, FR3, FR4, respectively; the asterisk indicates the position of an L→N mutation in VL FR4 as compared to the VL FR4 of palivizumab. This VL domain (SEQ ID NO:11) is identical to that of the MEDI-524 antibody described elsewhere herein. In some embodiments, this VL framework can be used in combination with any of the VL CDRs identified in Table 1. In one embodiment, the MEDI-524 antibody comprises the VL domain (SEQ ID NO:209) and the C-kappa constant domain described in Johnson et al. (1997) J. Infect. Dis. 176, 1215-1224 and U.S. Pat. No. 5,824,307, wherein said antibody comprises a modified IgG, such as a modified IgG1, constant domain, or FcRn-binding fragment thereof. In one embodiment, an Fc modified antibody of the invention comprises a VL chain having the amino acid sequence of SEQ ID NO:209 and/or a VL domain having the amino acid sequence of SEQ ID NO:8. In another embodiment, an Fc modified antibody of the invention comprises a VL chain having the amino acid sequence SEQ ID NO:255 and/or a VL domain having the amino acid sequence SEQ ID NO:11.

In a specific embodiment, Fc modified antibodies that immunospecifically bind to a RSV antigen (e.g., RSV F antigens) comprise a VH domain having the amino acid sequence of SEQ ID NO:7, SEQ ID NO:9, SEQ ID NO:17, SEQ ID NO:24, SEQ ID NO:28, SEQ ID NO:33, SEQ ID NO:36, SEQ ID NO:40, SEQ ID NO:44, SEQ ID NO:48, SEQ ID NO:51, SEQ ID NO:55, SEQ ID NO:67, SEQ ID NO:78, SEQ ID NO:304, SEQ ID NO:310, SEQ ID NO:317, SEQ ID NO:323, or SEQ ID NO:328, and a VL domain having the amino acid sequence of SEQ ID NO:8, SEQ ID NO:13, SEQ ID NO:21, SEQ ID NO:26, SEQ ID NO:30, SEQ ID NO:34, SEQ ID NO:38, SEQ ID NO:42, SEQ ID NO:46, SEQ ID NO:49, SEQ ID NO:52, SEQ ID NO:54, SEQ ID NO:56, SEQ ID NO:58, SEQ ID NO:60, SEQ ID NO:62, SEQ ID NO:64, SEQ ID NO:65, SEQ ID NO:68, SEQ ID NO:70, SEQ ID NO:71, SEQ ID NO:74, SEQ ID NO:76, SEQ ID NO:307, SEQ ID NO:313, SEQ ID NO:319, SEQ ID NO:325, SEQ ID NO:331, or SEQ ID NO:334. In a other embodiment, Fc modified antibodies that immunospecifically bind to a RSV F antigen comprise a VH domain having the amino acid sequence of SEQ ID NO:48 and a VL domain comprising the amino acid sequence of SEQ ID NO:11. In another specific embodiment, the Fc modified antibodies of the invention have a high affinity and/or high avidity for a RSV antigen (e.g., RSV F antigen).

In one embodiment, an Fc modified antibody of the invention comprises a VH CDR1 having the amino acid sequence of SEQ ID NO:1, SEQ ID NO:10 or SEQ ID NO:18 and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an Fc modified antibody of the invention comprises a VH CDR1 having the amino acid sequence of SEQ ID NO:1, SEQ ID NO:10 or SEQ ID NO:18 and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, an Fc modified antibody of the invention comprises a VH CDR1 having the amino acid sequence of SEQ ID NO:1, SEQ ID NO:10 or SEQ ID NO:18 and a VL CDR3 having the amino acid sequence of SEQ ID NO:6, SEQ ID NO:16 or SEQ ID NO:61. In accordance with these embodiments, the antibody immunospecifically binds to a RSV F antigen.

In another embodiment, an Fc modified antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an Fc modified antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In another embodiment, an Fc modified antibody of the invention comprises a VH CDR2 having the amino acid sequence of SEQ ID NO:2, SEQ ID NO:19, SEQ ID NO:25, SEQ ID NO:37, SEQ ID NO:41, SEQ ID NO:45, SEQ ID NO:305, or SEQ ID NO:329, and a VL CDR3 having the amino acid sequence of SEQ ID NO:6, SEQ ID NO:16, or SEQ ID NO:61. In accordance with these embodiments, the antibody immunospecifically binds to a RSV F antigen.

In another embodiment, an Fc modified antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR1 having the amino acid sequence of SEQ ID NO:4, SEQ ID NO:14, SEQ ID NO:22, SEQ ID NO:31, SEQ ID NO:39, SEQ ID NO:47, SEQ ID NO:314, SEQ ID NO:320, or SEQ ID NO:335. In another embodiment, an Fc modified antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR2 having the amino acid sequence of SEQ ID NO:5, SEQ ID NO:15, SEQ ID NO:23, SEQ ID NO:27, SEQ ID NO:32, SEQ ID NO:35, SEQ ID NO:43, SEQ ID NO:50, SEQ ID NO:53, SEQ ID NO:57, SEQ ID NO:59, SEQ ID NO:63, SEQ ID NO:66, SEQ ID NO:69, SEQ ID NO:73, SEQ ID NO:75, SEQ ID NO:77, SEQ ID NO:308, SEQ ID NO:315, SEQ ID NO:321, SEQ ID NO:326, SEQ ID NO:332, or SEQ ID NO:336. In a other embodiment, an Fc modified antibody of the invention comprises a VH CDR3 having the amino acid sequence of SEQ ID NO:3, SEQ ID NO:12, SEQ ID NO:20, SEQ ID NO:29, SEQ ID NO:79, or SEQ ID NO:311, and a VL CDR3 having the amino acid sequence of SEQ ID NO:6, SEQ ID NO:16, or SEQ ID NO:61. In accordance with these embodiments, the antibody immunospecifically binds to a RSV F antigen.

The present invention also provides Fc modified antibodies that immunospecifically bind to a RSV antigen (e.g., RSV F antigen), the Fc modified antibodies comprising derivatives of the VH domains, VH CDRs, VL domains, and VL CDRs described herein that immunospecifically bind to a RSV antigen. The present invention also provides antibodies comprising derivatives of palivizumab, AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 as shown in Table 1, comprising a modified IgG (e.g., IgG1) constant domain, or FcRn binding fragment thereof (e.g., the Fc domain or hinge-Fc domain), described herein and wherein said antibodies immunospecifically bind to one or more RSV antigens (e.g., RSV F antigen).

The present invention also provides Fc modified antibodies that immunospecifically bind to a RSV antigen (e.g., RSV F antigen) which comprise a framework region known to those of skill in the art (e.g., a human or non-human fragment). The framework region may be naturally occurring or consensus framework regions. Preferably, the framework region of an antibody of the invention is human (see, e.g., Chothia et al., 1998, J. Mol. Biol. 278:457-479 for a listing of human framework regions, which is incorporated by reference herein in its entirety). In a specific embodiment, an antibody of the invention comprises the framework region of MEDI-524.

In a specific embodiment, the present invention provides for Fc modified antibodies that immunospecifically bind to a RSV F antigen, said antibodies comprising the amino acid sequence of one or more of the CDRs of an antibody listed in Table 1 (i.e., AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 and/or one or more of the CDRs in Table 1, and human framework regions with one or more amino acid substitutions at one, two, three or more of the following residues: (a) rare framework residues that differ between the murine antibody framework (i.e., donor antibody framework) and the human antibody framework (i.e., acceptor antibody framework); (b) Venier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In certain embodiments, the above-referenced antibodies comprise a modified IgG (e.g., IgG1) constant domain, or FcRn binding fragment thereof (e.g., the Fc domain or hinge-Fc domain), described herein.

The present invention encompasses Fc modified antibodies that immunospecifically bind to a RSV F antigen, said antibodies comprising the amino acid sequence of the VH domain and/or VL domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 as shown in Table 1 with mutations (e.g., one or more amino acid substitutions) in the framework regions. In certain embodiments, antibodies that immunospecifically bind to a RSV antigen comprise the amino acid sequence of the VH domain and/or VL domain or an antigen-binding fragment thereof of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 as shown in Table 1 with one or more amino acid residue substitutions in the framework regions of the VH and/or VL domains.

The present invention also encompasses antibodies which immunospecifically bind to one or more RSV antigens (e.g., RSV F antigens), said antibodies comprising the amino acid sequence of MEDI-524 with mutations (e.g., one or more amino acid substitutions) in the framework regions. In certain embodiments, antibodies which immunospecifically bind to one or more RSV F antigens comprise the amino acid sequence of MEDI-524 with one or more amino acid residue substitutions in the framework regions of the VH and/or VL domains and one or more modifications in the constant domain, or FcRn-binding fragment thereof (preferably the Fc domain or hinge-Fdc domain).

The present invention also encompasses Fc modified antibodies that immunospecifically bind to a RSV antigen, said antibodies comprising the amino acid sequence of the VH domain and/or VL domain of an antibody in Table 1 (i.e., AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4) with mutations (e.g., one or more amino acid residue substitutions) in the hypervariable and framework regions. Preferably, the amino acid substitutions in the hypervariable and framework regions improve binding of the antibody to a RSV antigen.

The present invention also provides for fusion proteins comprising an antibody provided herein that immunospecifically binds to a RSV antigen and a heterologous polypeptide. Preferably, the heterologous polypeptide that the antibody are fused to is useful for targeting the antibody to respiratory epithelial cells.

5.1.1 Modifications of Antibody Fc Regions

The present invention provides for modified antibodies that immunospecifically bind to a RSV antigen which have modifications to their Fc regions.

In certain embodiments, the in vivo half-life of the modified antibody is increased as compared to as compared to the same antibody that does not comprise one or more modifications in the IgG constant domain, or FcRn-binding fragment thereof, as determined using methods described herein or known in the art (see Example 6.17). In some embodiments, the half-life of the modified antibody is increased by about 2-fold, about 3-fold, about 4-fold, about 5-fold, about 6-fold, about 7-fold, about 8-fold, about 9-fold, about 10-fold, about 20-fold or more as compared to the same antibody that does not comprise one or more modifications in the IgG constant domain, or FcRn-binding fragment thereof. In certain embodiments, the half-life of the modified antibody is increased by 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days or more as compared to the same antibody that does not comprise one or more modifications in the IgG constant domain, or FcRn-binding fragment thereof.

In a specific embodiment, modified antibodies having an increased half-life in vivo are be generated by introducing one or more amino acid modifications (i.e., substitutions, insertions or deletions) into an IgG constant domain, or FcRn-binding fragment thereof (preferably a Fc or hinge-Fc domain fragment). See, e.g., International Publication Nos. WO 02/060919; WO 98/23289; and WO 97/34631; and U.S. Pat. No. 6,277,375; each of which is incorporated herein by reference in its entirety. In a other embodiment, the modified antibodies have one or more amino acid modifications in the second constant CH2 domain (residues 231-340 of human IgG1) (e.g., SEQ ID NO:339) and/or the third constant CH3 domain (residues 341-447 of human IgG1) (e.g., SEQ ID NO:340), with numbering according to the EU Index as in Kabat, supra.

The present invention provides amino acid residues and/or modifications in particular portions of the constant domain (e.g., of an IgG molecule) that interact with the FcRn, which modifications increase the affinity of the IgG, or fragment thereof, for the FcRn. Accordingly, the invention provides molecules, preferably proteins, more preferably immunoglobulins (including any antibody disclosed in this application), that comprise an IgG (e.g., IgG1) constant domain, or FcRn-binding fragment thereof (preferably a Fc or hinge-Fc domain fragment), having one or more amino acid modifications (i.e., substitutions, insertions, deletions, and/or naturally occurring residues) in one or more regions that interact with the FcRn, which modifications increase the affinity of the IgG or fragment thereof, for the FcRn, and also increase the in vivo half-life of the molecule. In certain embodiments, the one or more amino acid modifications are made in one or more of residues 251-256, 285-290, 308-314, 385-389, and 428-436 of the IgG hinge-Fc region (for example, as in the human IgG1 hinge-Fc region depicted in SEQ ID NO:342), or analogous residues thereof, as determined by amino acid sequence alignment, in other IgG hinge-Fc regions. Numbering of residues are according to the EU index in Kabat et al. (1991). Sequences of proteins of immunological interest. (U.S. Department of Health and Human Services, Washington, D.C.) 5^{th} ed. (“Kabat et al.”). Antibody modifications are described in co-owned and co-pending U.S. Ser. No. 10/020,354 which is incorporated herein by reference in its entirety.

In another embodiment, the amino acid modifications are made in a human IgG constant domain such as a human IgG1 constant domain (e.g., those described in Kabat et al., supra), or FcRn-binding fragment thereof (preferably, Fc domain or hinge-Fc domain). In a certain embodiment, the modifications are not made at residues 252, 254, or 256 (i.e., all are made at one or more of residues 251, 253, 255, 285-290, 308-314, 385-389, or 428-436) of the IgG constant domain. In one embodiment, the amino acid modifications are not the substitution with leucine at residue 252, with serine at 254, and/or with phenylalanine at position 256. In particular, in certain embodiments, such modifications are not made when the IgG constant domain, hinge-Fc domain, hinge-Fc domain or other FcRn-binding fragment thereof is derived from a mouse.

The amino acid modifications may be any modification, for example, at one or more of residues 251-256, 285-290, 308-314, 385-389, and 428-436, that increases the in vivo half-life of the IgG constant domain, or FcRn-binding fragment thereof (e.g., Fc or hinge-Fc domain), and any molecule attached thereto, and increases the affinity of the IgG, or fragment thereof, for FcRn. In some embodiments, the modified antibodies comprise one or more amino acid substitutions, naturally occurring amino acids, or combinations thereof, at the indicated amino acid positions. Preferably, the one or more modifications also result in a higher binding affinity of the constant domain, or FcRn-binding fragment thereof, for FcRn at pH 6.0 than at pH 7.4. In other embodiments, the modifications alter (i.e., increase or decrease) bioavailability of the molecule, in particular, alters (i.e., increases or decreases) transport (or concentration or half-life) of the molecule to mucosal surfaces (e.g., of the lungs) or other portions of a target tissue. In another embodiment, the amino acid modifications alter (preferably, increase) transport or concentration or half-life of the molecule to the lungs. In other embodiments, the amino acid modifications alter (preferably, increase) transport (or concentration or half-life) of the molecule to the heart, pancreas, liver, kidney, bladder, stomach, large or small intestine, respiratory tract, lymph nodes, nervous tissue (central and/or peripheral nervous tissue), muscle, epidermis, bone, cartilage, joints, blood vessels, bone marrow, prostate, ovary, uterine, tumor or cancer tissue, etc.

In certain embodiments, the IgG constant domain comprises a modification at one or more of residues 308, 309, 311, 312 and 314. In some embodiments, a modified antibody comprises a threonine at position 308, proline at position 309, serine at position 311, aspartic acid at position 312, and/or leucine at position 314. In other embodiments, a modified antibody comprises an isoleucine at position 308, proline at position 309, and/or a glutamic acid at position 311. In yet another embodiment, a modified antibody comprises a threonine at position 308, a proline at position 309, a leucine at position 311, an alanine at position 312, and/or an alanine at position 314. Accordingly, in certain embodiments a modified antibody comprises a constant domain, wherein the residue at position 308 is a threonine or isoleucine, the residue at position 309 is proline, the residue at position 311 is serine, glutamic acid or leucine, the residue at position 312 is alanine, and/or the residue at position 314 is leucine or alanine. In one embodiment, a modified antibody comprises threonine at position 308, proline at position 309, serine at position 311, aspartic acid at position 312, and/or leucine at position 314.

In some embodiments, a modified antibody comprises a constant domain, wherein one or more of residues 251, 252, 254, 255, and 256 is modified. In specific embodiments, residue 251 is leucine or arginine, residue 252 is tyrosine, phenylalanine, serine, tryptophan or threonine, residue 254 is threonine or serine, residue 255 is arginine, leucine, glycine, or isoleucine, and/or residue 256 is serine, arginine, glutamine, glutamic acid, aspartic acid, alanine, asparagine or threonine. In a more specific embodiment, residue 251 is leucine, residue 252 is tyrosine, residue 254 is threonine or serine, residue 255 is arginine, and/or residue 256 is glutamic acid. In certain embodiments, the residue at position 252 is a tyrosine, the residue at position 254 is a threonine, or the residue at position 256 is a glutamic acid. In other embodiments, modified IgG, such as a modified IgG1, constant domain, or FcRn binding fragment thereof, comprises the YTE modification, i.e., the residue at position 252 is a tyrosine (Y), the residue at position 254 is a threonine (T), and the residue at position 256 is a glutamic acid (E).

In specific embodiments, the amino acid modifications are substitutions at one or more of residues 428, 433, 434, and 436. In some embodiments, residue 428 is threonine, methionine, leucine, phenylalanine, or serine, residue 433 is lysine, arginine, serine, isoleucine, proline, glutamine or histidine, residue 434 is phenylalanine, tyrosine, or histidine, and/or residue 436 is histidine, asparagine, arginine, threonine, lysine, or methionine. In a more specific embodiment, residues at position 428 and/or 434 are substituted with methionine, and/or histidine respectively.

In other embodiments, the amino acid sequence comprises modifications at one or more of residues 385, 386, 387, and 389. In specific embodiments, residue 385 is arginine, aspartic acid, serine, threonine, histidine, lysine, alanine or glycine, residue 386 is threonine, proline, aspartic acid, serine, lysine, arginine, isoleucine, or methionine, residue 387 is arginine, proline, histidine, serine, threonine, or alanine, and/or residue 389 is proline, serine or asparagine. In more specific embodiments, one or more of positions 385, 386, 387, and 389 are arginine, threonine, arginine, and proline, respectively. In yet another specific embodiment, one or more of positions 385, 386, and 389 are aspartic acid, proline, and serine, respectively.

In some embodiments, amino acid modifications are made at one or a combination of residues 251, 252, 254, 255, 256, 308, 309, 311, 312, 314, 385, 386, 387, 389, 428, 433, 434, and/or 436, particularly where the modifications are amino acid residues described immediately above for these residues.

In some embodiments, the molecule of the invention contains a Fc region, or FcRn-binding fragment thereof, having one or more of the following: leucine at residue 251, tyrosine at residue 252, threonine or serine at residue 254, arginine at residue 255, threonine at residue 308, proline at residue 309, serine at residue 311, aspartic acid at residue 312, leucine at residue 314, arginine at residue 385, threonine at residue 386, arginine at residue 387, proline at residue 389, methionine at residue 428, and/or tyrosine at residue 434.

In certain embodiments, the FcRn-binding fragment has a modification at 1, 2, 3, 4, 5, 6, 7, 8, 9, 10, 11, 12, 13, 14, 15, 16, 17, or all 18 of residues 251, 252, 254, 255, 256, 308, 309, 311, 312, 314, 385, 386, 387, 389, 428, 433, 434, and/or 436.

Due to natural variations in IgG constant domain sequences (see, e.g., Kabat et al., supra), in certain instances, a first amino acid residue may be substituted (or otherwise modified) with a second amino acid residue at a given position or, alternatively, the second residue may be already present in antibody at the given position, in which case substitution is not necessary (for example, the Met at position 252 remains a Met). Amino acid modifications can be made by any method known in the art and many such methods are well known and routine for the skilled artisan. For example, but not by way of limitation, amino acid substitutions, deletions and insertions may be accomplished using any well-known PCR-based technique. Amino acid substitutions may be made by site-directed mutagenesis (see, for example, Zoller and Smith, Nucl. Acids Res. 10:6487-6500, 1982; Kunkel, Proc. Natl. Acad. Sci. USA 82:488, 1985, which are hereby incorporated by reference in their entireties). Mutants that result in increased affinity for FcRn and increased in vivo half-life may readily be screened using well-known and routine assays. In a preferred method, amino acid substitutions are introduced at one or more residues in the IgG constant domain or FcRn-binding fragment thereof and the mutated constant domains or fragments are expressed on the surface of bacteriophage which are then screened for increased FcRn binding affinity.

Preferably, the modified amino acid residues are surface exposed residues. Additionally, in making amino acid substitutions, preferably the amino acid residue to be substituted is a conservative amino acid substitution, for example, a polar residue is substituted with a polar residue, a hydrophilic residue with a hydrophilic residue, hydrophobic residue with a hydrophobic residue, a positively charged residue with a positively charged residue, or a negatively charged residue with a negatively charged residue. Moreover, preferably, the modified amino acid residue is not highly or completely conserved across species and/or is critical to maintain the constant domain tertiary structure or to FcRn binding. For example, but not by way of limitation, modification of the histidine at residue 310 is not preferred.

Specific mutants of the Fc domain that have increased affinity for FcRn were isolated after the third-round panning from a library of mutant human IgG1 molecules having mutations at residues 308-314 (histidine at position 310 and tryptophan at position 313 are fixed), those isolated after the fifth-round panning of the library for residues 251-256 (isoleucine at position 253 is fixed), those isolated after fourth-round panning of the library for residues 428-436 (histidine at position 429, glutamic acid at position 430, alanine at position 431, leucine at position 432, and histidine at position 435 are fixed), and those isolated after sixth-round panning of the library for residues 385-389 (glutamic acid at position 388 is fixed).

In some embodiments, an antibody of the invention contains a Fc region, or FcRn-binding fragment thereof, having one or more amino acid modifications. Preferably, the one or more amino acid modifications may be substitutions. In one embodiment, the one or more amino acid substitutions are: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat. Such Fc domain amino acid substitutions encompass an increase in ADCC (3M) if compared to the same antibody without said amino acid substitutions. A specific embodiment for 3M includes, but is not limited to, 239D, 330L, and 332E. In another embodiment, the one or more amino acid modifications are, in addition to those described for 3M, in combination with those at positions 251-256, 285-290, 308-314, 385-389, and 428-436, with numbering according to the EU Index as in Kabat. Such Fc domain combination amino acid substitutions encompass a modified antibody having either an increase in ADCC (3M) with an increase in in vivo half life, if both are compared to the same antibody without said amino acid substitutions. In certain embodiments, an IgG constant domain comprises a 239D, 330L, 332E, 252Y, 254T, and 256E. Among the amino acid residues at positions 251-256 of the Fc region selected from the group consisting of the following residues: residue 252 is tyrosine, phenylalanine, serine, tryptophan or threonine; residue 254 is threonine; residue 255 is arginine, leucine, glycine, or isoleucine; and residue 256 is serine, arginine, glutamine, glutamic acid, aspartic acid, or threonine. In a particular embodiment, at least one amino acid modification is selected from the group consisting of the following: residue 251 is leucine, residue 252 is tyrosine, residue 254 is threonine, residue 255 is arginine, and residue 256 is glutamic acid. In certain embodiments, residue 252 is not leucine, alanine, or valine; residue 253 is not alanine; residue 254 is not serine or alanine; residue 255 is not alanine; and/or residue 256 is not alanine, histidine, phenylalanine, glycine, or asparagine.

In another embodiment, a modified antibody of the invention contains a Fc region, or FcRn-binding fragment thereof, having one or more particular amino acid residues among the amino acid residues at positions 285-290 of the Fc region. In particular embodiments, residue 285 is not alanine; residue 286 is not alanine, glutamine, serine, or aspartic acid; residue 288 is not alanine; residue 289 is not alanine; and/or residue 290 is not alanine, glutamine, serine, glutamic acid, arginine, or glycine.

In some embodiments, a modified antibody of the invention contains a Fc region, or FcRn-binding fragment thereof, having one or more particular amino acid residues among the amino acid residues at positions 308-314 of the Fc region selected from the group consisting of the following residues: a threonine at position 308, a proline at position 309, a serine at position 311, and an aspartic acid at position 312. In another embodiment, an antibody of the invention comprises one or more specific modifications selected from the group consisting of an isoleucine at position 308, a proline at position 309, and a glutamic acid at position 311. In another embodiment, a modified antibody comprises one or more specific amino acid residues selected from the group consisting of a threonine at position 308, a proline at position 309, and a leucine at position 311. In certain embodiments, position 309 is not an alanine; position 310 is not an alanine; position 311 is not an alanine or an asparagine; position 312 is not an alanine; and/or position 314 is not an arginine.

Accordingly, in certain embodiments a modified antibody comprises a constant domain having one or more particular amino acid residues in the Fc region selected from the group consisting of the following residues: the residue at position 308 is threonine or isoleucine; the residue at position 309 is proline; the residue at position 311 is serine, glutamic acid or leucine; the residue at position 312 is aspartic acid; and the residue at position 314 is leucine or alanine. In an embodiment, the modified antibody comprises a constant domain having one or more particular amino acid residues in the Fc region selected from the group consisting of the following residues: threonine at position 308, proline at position 309, serine at position 311, aspartic acid at position 312, and leucine at position 314.

In some embodiments, an antibody of the invention contains a Fc region, or FcRn-binding fragment thereof, having one or more particular amino acid residues among the amino acid residues at positions 385-389 of the Fc region selected from the group consisting of the following residues: residue 385 is arginine, aspartic acid, serine, threonine, histidine, lysine, alanine or glycine; residue 386 is threonine, proline, aspartic acid, serine, lysine, arginine, isoleucine, or methionine; residue 387 is arginine, proline, histidine, serine, threonine, or alanine; and residue 389 is proline, serine or asparagine. In particular embodiments, one or more of the amino acid residue at positions 385, 386, 387, and 389 is arginine, threonine, arginine, and proline, respectively. In another specific embodiment, one or more of the amino acid residues at positions 385, 386, and 389 is aspartic acid, proline, and serine, respectively. In particular embodiments, the amino acid at any one of positions 386, 388, and 389 is not an alanine.

In some embodiments, the amino acid modifications are at one or more of residues 428-436. In specific embodiments, residue 428 is threonine, methionine, leucine, phenylalanine, or serine, residue 433 is arginine, serine, isoleucine, proline, glutamine or histidine, residue 434 is phenylalanine, tyrosine, or histidine, and/or residue 436 is histidine, asparagine, arginine, threonine, lysine, or methionine. In a more specific embodiment, residues at position 428 and/or 434 are substituted with methionine, and/or histidine respectively. In some embodiments, the amino acid residue at position 430 is not alanine; the amino acid residue at position 433 is not alanine or lysine; the amino acid at position 434 is not alanine or glutamine; the amino acid at position 435 is not alanine, arginine, or tyrosine; and/or the amino acid at position 436 is not alanine or tyrosine.

In another embodiment, an antibody of the invention contains a Fc region, or FcRn-binding fragment thereof, having one or more particular amino acid residues in the Fc region selected from the group consisting of a leucine at residue 251, a tyrosine at residue 252, a threonine at residue 254, an arginine at residue 255, a threonine at residue 308, a proline at residue 309, a serine at residue 311, an aspartic acid at residue 312, a leucine at residue 314, an arginine at residue 385, a threonine at residue 386, an arginine at residue 387, a proline at residue 389, a methionine at residue 428, and a tyrosine at residue 434.

In one embodiment, the invention provides modified immunoglobulin molecules that have increased in vivo half-life and affinity for FcRn relative to unmodified molecules (and, in some embodiments, altered bioavailability such as increased or decreased transport to mucosal surfaces or other target tissues). Such immunoglobulin molecules include IgG molecules that naturally contain an FcRn-binding fragment and other non-IgG immunoglobulins (e.g., IgE, IgM, IgD, IgA and IgY) or fragments of immunoglobulins that have been engineered to contain an FcRn-binding fragment (i.e., fusion proteins comprising non-IgG immunoglobulin or a portion thereof and an FcRn-binding fragment). In both cases the FcRn-binding fragment has one or more amino acid modifications that increase the affinity of the constant domain fragment for FcRn, such as those provided above.

The modified immunoglobulins include any immunoglobulin molecule that binds (preferably, immunospecifically, i.e., competes off non-specific binding), as determined by immunoassays well known in the art and described herein for assaying specific antigen-antibody binding an antigen and contains an FcRn-binding fragment.

The IgG molecules of the invention, and FcRn-binding fragments thereof, are preferably IgG1 subclass of IgGs, but may also be any other IgG subclasses of given animals. For example, in humans, the IgG class includes IgG1, IgG2, IgG3, and IgG4; and mouse IgG includes IgG1, IgG2a, IgG2b, IgG2c and IgG3. It is known that certain IgG subclasses, for example, mouse IgG2b and IgG2c, have higher clearance rates than, for example, IgG1 (Medesan et al., Eur. J. Immunol., 28:2092-2100, 1998). Thus, when using IgG subclasses other than IgG1, it may be advantageous to substitute one or more of the residues, particularly in the CH2 and CH3 domains, that differ from the IgG1 sequence with those of IgG1, thereby increasing the in vivo half-life of the other types of IgG.

The immunoglobulins (and other proteins used herein) may be from any animal origin including birds and mammals. In one embodiment, the antibodies are human, rodent (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598 by Kucherlapati et al.

Modification of any of the antibodies of the invention (e.g., those with increased affinity and/or avidity for a RSV antigen) and/or other therapeutic antibodies to increase the in vivo half-life permits administration of lower effective dosages and/or less frequent dosing of the therapeutic antibody. Such modification to increase in vivo half-life can also be useful to improve diagnostic immunoglobulins as well, for example, permitting administration of lower doses to achieve sufficient diagnostic sensitivity.

One or more modifications in amino acid residues 251-256, 285-290, 308-314, 385-389, and 428-436 of the constant domain may be introduced utilizing any technique known to those of skill in the art. The constant domain or fragment thereof having one or more modifications in amino acid residues 251-256, 285-290, 308-314, 385-389, and 428-436 may be screened by, for example, a binding assay to identify the constant domain or fragment thereof with increased affinity for the FcRn receptor (e.g., as described in Sections 5.5 and 5.6, infra). Those modifications in the hinge-Fc domain or the fragments thereof which increase the affinity of the constant domain or fragment thereof for the FcRn receptor can be introduced into antibodies to increase the in vivo half-lives of said antibodies. Further, those modifications in the constant domain or the fragment thereof which increase the affinity of the constant domain or fragment thereof for the FcRn can be fused to bioactive molecules to increase the in vivo half-lives of said bioactive molecules (and, preferably alter (increase or decrease) the bioavailability of the molecule, for example, to increase or decrease transport to mucosal surfaces (or other target tissue) (e.g., the lungs).

5.1.2 Antibody Conjugates and Fusion Proteins

In some embodiments, antibodies of the invention are conjugated or recombinantly fused to a diagnostic, detectable or therapeutic agent or any other molecule. When in vivo half-life is desired to be increased, said antibodies can be modified antibodies. The conjugated or recombinantly fused antibodies can be useful, e.g., for monitoring or prognosing the onset, development, progression and/or severity of a RSV URI and/or LRI as part of a clinical testing procedure, such as determining the efficacy of a particular therapy.

Further, an antibody of the invention may be conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, γ-interferon, α-interferon, nerve growth factor, platelet derived growth factor, tissue plasminogen activator, an apoptotic agent, e.g., TNF-γ, TNF-γ, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma, interleukin-1 (“IL-1”), interleukin-2 (“IL-2”), interleukin-5 (“IL-5”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

The present invention encompasses antibodies of the invention (e.g., modified antibodies) recombinantly fused or chemically conjugated (including both covalent and non-covalent conjugations) to a heterologous protein or polypeptide (or fragment thereof, preferably to a polypeptide of about 10, about 20, about 30, about 40, about 50, about 60, about 70, about 80, about 90 or about 100 amino acids) to generate fusion proteins. In particular, the invention provides fusion proteins comprising an antigen-binding fragment of an antibody of the invention (e.g., a Fab fragment, Fd fragment, Fv fragment, F(ab)₂ fragment, a VH domain, a VH CDR, a VL domain or a VL CDR) and a heterologous protein, polypeptide, or peptide. Preferably, the heterologous protein, polypeptide, or peptide that the antibody is fused to is useful for targeting the antibody to a particular cell type. For example, an antibody that immunospecifically binds to a cell surface receptor expressed by a particular cell type (e.g., an immune cell) may be fused or conjugated to a modified antibody of the invention.

In one embodiment, a fusion protein of the invention comprises AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 antibody and a heterologous polypeptide. In another embodiment, a fusion protein of the invention comprises an antigen-binding fragment of AFFF, P12f2, P12f4, P11d4, A1e9, A12a6, A13c4, A17d4, A4B4, A8C7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 and a heterologous polypeptide. In another embodiment, a fusion protein of the invention comprises one or more VH domains having the amino acid sequence of any one of the VH domains listed in Table 1 or one or more VL domains having the amino acid sequence of any one of the VL domains listed in Table 1 and a heterologous polypeptide. In another embodiment, a fusion protein of the present invention comprises one or more VH CDRs having the amino acid sequence of any one of the VH CDRs listed in Table 1 and a heterologous polypeptide. In another embodiment, a fusion protein comprises one or more VL CDRs having the amino acid sequence of any one of the VL CDRs listed in Table 1 and a heterologous polypeptide. In another embodiment, a fusion protein of the invention comprises at least one VH domain and at least one VL domain listed in Table 1 and a heterologous polypeptide. In yet another embodiment, a fusion protein of the invention comprises at least one VH CDR and at least one VL CDR domain listed in Table 1 and a heterologous polypeptide.

In addition, an antibody of the invention can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹W, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; -C-U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992; and U.S. Provisional Application No. 60/727,043 filed Oct. 14, 2005 entitled “Methods of Preventing and Treating RSV Infections and Related Conditions;” and U.S. Provisional No. 60/727,042 filed Oct. 14, 2005 by Genevieve Losonsky entitled “Methods of Administering/Dosing Anti-RSV Antibodies for Prophylaxis and Treatment of RSV Infections and Respiratory Conditions;” which are incorporated herein by reference in their entireties.

In particular, fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies of the invention (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an antibody of the invention may be recombined with one or more components, motifs, sections, parts, domains, fragments, etc. of one or more heterologous molecules.

The therapeutic moiety or drug conjugated or recombinantly fused to an antibody of the invention that immunospecifically binds to a RSV antigen should be chosen to achieve the desired therapeutic effect(s). A clinician or other medical personnel should consider the following when deciding on which therapeutic moiety or drug to conjugate or recombinantly fuse to an antibody of the invention: the nature of the disease, the severity of the disease, and the condition of the subject.

5.2 Therapeutic Uses of Antibodies

The present invention is directed to antibody-based therapies which involve administering antibodies of the invention to a subject, preferably a human, (e.g., to a subject in need thereof) for managing, treating and/or ameliorating a RSV infection (e.g., acute RSV disease, or a RSV URI and/or LRI), and/or a symptom or respiratory condition relating thereto (e.g., asthma, wheezing, and/or RAD). Therapeutic agents of the invention include, but are not limited to, antibodies of the invention (including analogs and derivatives thereof as described herein) and nucleic acids encoding the antibodies of the invention (including analogs and derivatives thereof and anti-idiotypic antibodies as described herein). Antibodies of the invention may be provided in pharmaceutically acceptable compositions as known in the art or as described herein.

Antibodies of the present invention that function as antagonists of a RSV infection can be administered to a subject, preferably a human, to treat or ameliorate a RSV URI and/or LRI, or a symptom or respiratory condition relating thereto (including, but not limited to, asthma, wheezing, RAD, or a combination thereof). For example, antibodies that disrupt or prevent the interaction between a RSV antigen and its host cell receptor may be administered to subject, preferably a human, to prevent, manage, treat and/or ameliorate a RSV infection (e.g., acute RSV disease, or a RSV URI and/or LRI), and/or a symptom or respiratory condition relating thereto (e.g., asthma, wheezing, and/or RAD).

In a specific embodiment, an antibody of the invention prevents or inhibits RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibody or in the presence of a negative control in an assay known to one of skill in the art or described herein, such as by a competition assay or microneutralization assay. In another embodiment, a combination of antibodies of the invention prevents or inhibits RSV from binding to its host cell receptor by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV binding to its host cell receptor in the absence of said antibodies or in the presence of a negative control in an assay known to one of skill in the art or described herein. In certain embodiments, one or more modified antibodies of the invention can be administered either alone or in combination. In some embodiments, a combination of antibodies of the invention act synergistically to prevent or inhibit RSV from binding to its host and receptor and/or in managing, treating and/or ameliorating a RSV infection (e.g., acute RSV disease, or a RSV URI and/or LRI), and/or a symptom or respiratory condition relating thereto (e.g., asthma, wheezing, and/or RAD).

In a specific embodiment, an antibody of the invention (modified) prevents or inhibits RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibody or in the presence of a negative control in an assay known to one of skill in the art or described herein. In another embodiment, a combination of antibodies of the invention prevents or inhibits RSV-induced fusion by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV-induced fusion in the absence of said antibodies or in the presence of a negative control in an assay known to one of skill in the art or described herein.

In some embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lung. The fold-reduction in RSV titer may be as compared to a negative control (such as placebo), as compared to another treatment (including, but not limited to treatment with palivizumab), or as compared to the titer in the patient prior to antibody administration.

In a specific embodiment, an antibody of the present invention inhibits or downregulates RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibody or in the presence of a negative control in an assay known in the art or described herein. In another embodiment, a combination of antibodies of the invention inhibits or downregulates RSV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibodies or in the presence of a negative control in an assay known in the art or described herein.

In some embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the upper respiratory tract. The fold-reduction in RSV titer may be as compared to a negative control (such as placebo), as compared to another treatment (including, but not limited to treatment with palivizumab), or as compared to the titer in the patient prior to antibody administration. In other embodiments, an antibody of the invention results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in RSV titer in the lower respiratory tract. The fold-reduction in RSV titer may be as compared to a negative control (such as placebo), as compared to another treatment (including, but not limited to treatment with palivizumab), or as compared to the titer in the patient prior to antibody administration. The antibodies of the invention may be administered alone or in combination with other types of therapies (e.g., hormonal therapy, immunotherapy, and anti-inflammatory agents). In some embodiments, the antibodies of the invention act synergistically with the other therapies. Generally, administration of products of a species origin or species reactivity (in the case of antibodies) that is the same species as that of the patient is preferred. Thus, in a other embodiment, human or humanized antibodies, derivatives, analogs, or nucleic acids, are administered to a human patient for therapy.

It is possible to use high affinity and/or potent in vivo inhibiting antibodies and/or neutralizing antibodies that immunospecifically bind to a RSV antigen, for both immunoassays directed to RSV, and the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. It is also possible to use polynucleotides encoding high affinity and/or potent in vivo inhibiting antibodies and/or neutralizing antibodies that immunospecifically bind to a RSV antigen, for both immunoassays directed to RSV and therapy for a RSV infection (e.g., treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof). Such antibodies will preferably have an affinity for the RSV F glycoprotein and/or fragments of the F glycoprotein.

In one embodiment, the invention also provides methods of treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof as alternatives to current therapies. In a specific embodiment, the current therapy has proven or may prove to be too toxic (i.e., results in unacceptable or unbearable side effects) for the patient. In another embodiment, an antibody of the invention decreases the side effects as compared to the current therapy. In another embodiment, the patient has proven refractory to a current therapy. In such embodiments, the invention provides for the administration of one or more antibodies of the invention without any other anti-infection therapies. In certain embodiments, a patient with a RSV infection (e.g., acute RSV disease or RSV URI and/or LRI), is refractory to a therapy when the infection has not significantly been eradicated and/or the symptoms have not been significantly alleviated. The determination of whether a patient is refractory can be made either in vivo or in vitro by any method known in the art for assaying the effectiveness of a therapy for infections, using art-accepted meanings of “refractory” in such a context. In various embodiments, a patient with a RSV infection (e.g., acute RSV disease or RSV URI and/or LRI) is refractory when viral replication has not decreased or has increased following therapy.

In a specific embodiment, the invention provides methods for managing, treating, and/or ameliorating one or more secondary responses to a primary viral infection, said methods comprising administering an effective amount of one or more antibodies of the invention alone or in combination with an effective amount of other therapies (e.g., other therapeutic agents). Examples of secondary responses to a primary viral infection include, but are not limited to, asthma-like responsiveness to mucosal stimula, elevated total respiratory resistance, increased susceptibility to secondary viral, bacterial, and fungal infections, and development of conditions such as, but not limited to, bronchiolitis, pneumonia, croup, and febrile bronchitis.

In other embodiments, a modified antibody of the invention can be used in passive immunotherapy (for therapy). To the extent the modified antibody also encompasses an extended half-life Fc modification, passive immunotherapy can be accomplished using lower doses and/or less frequent administration of the antibody resulting in fewer side effects, better patient compliance, less costly therapy/prophylaxis, etc. In a other embodiment, the therapeutic is an antibody that binds RSV, for example, any one or more of the anti-RSV antibodies described herein, wherein said antibody is a modified antibody. In certain embodiments, antibodies of the invention can be used in passive immunotherapy.

In other embodiments, a human patient who is infected with RSV is treated by administering to said patient in need thereof a therapeutically effective amount of a F(ab)′ fragment comprising three variable heavy complementarity determining regions (VH CDRs) and three variable light CDRs (VL CDRs) having an amino acid sequence of VH CDR 1 (SEQ ID NO:10), VH CDR 2 (SEQ ID NO:19), and VH CDR 3 (SEQ ID NO:20) and having an amino acid sequence of VL CDR 1 (SEQ ID NO:39), VL CDR 2 (SEQ ID NO:5), and VL CDR 3 (SEQ ID NO:6), wherein said administration is pulmonary and is during the RSV season. There typically occurs a “spike” of RSV infections and/or RSV disease during the height of RSV season in adults and in the elderly. It is contemplated that a method of treatment with the above F(ab)′ fragment can reduce the number of patient hospitalizations due to COPD, as compared to a similar cohort of patients who did not receive a therapeutically effective amount of said F(ab)′ fragment or placebo.

5.3 Methods of Administration, Frequency, and Dosing of Antibodies

In an embodiment, a composition for use in the management, treatment and/or amelioration of a RSV infection (e.g., acute RSV disease, or a RSV URI and/or LRI), and/or a symptom or respiratory condition relating thereto (e.g., asthma, wheezing, and/or RAD) comprises MEDI-524 comprising a modified IgG (e.g., IgG1) constant domain, or FcRn binding fragment thereof (e.g., the Fc domain or hinge-Fc domain), described herein. In yet another embodiment, a composition of the present invention comprises one or more fusion proteins of the invention.

Various delivery systems are known and can be used to administer a therapeutic agent (e.g., a modified antibody of the invention), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. Methods of administering a therapeutic agent (e.g., an antibody of the invention), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a therapeutic agent (e.g., an antibody of the present invention), or a pharmaceutical composition is administered intranasally, intramuscularly, intravenously, or subcutaneously. The therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an, aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a specific embodiment, an antibody of the invention, or composition of the invention is administered using Alkermes AIR™ pulmonary drug delivery technology (Alkermes, Inc., Cambridge, Mass.).

In a specific embodiment, it may be desirable to administer a therapeutic agent, or a pharmaceutical composition of the invention locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. Preferably, when administering an antibody of the invention, care must be taken to use materials to which the antibody does not absorb.

In a specific embodiment, a composition of the invention comprises one, two or more antibodies of the invention. In another embodiment, a composition of the invention comprises one, two or more antibodies of the invention and a therapeutic agent other than an antibody of the invention. Preferably, the agents are known to be useful for or have been or are currently used for the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. In addition to therapeutic agents, the compositions of the invention may also comprise a carrier.

The compositions of the invention include bulk drug compositions useful in the manufacture of pharmaceutical compositions (e.g., compositions that are suitable for administration to a subject or patient) that can be used in the preparation of unit dosage forms. In another embodiment, a composition of the invention is a pharmaceutical composition. Such compositions comprise a therapeutically effective amount of one or more therapeutic agents (e.g., a modified antibody of the invention or other therapeutic agent), and a pharmaceutically acceptable carrier. Preferably, the pharmaceutical compositions are formulated to be suitable for the route of administration to a subject.

In a specific embodiment, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a other carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody, preferably in purified form, together with a suitable amount of carrier so as to provide the form for proper administration to the patient. The formulation should suit the mode of administration.

In another embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides that an antibody of the invention is packaged in a hermetically sealed container such as an ampoule or sachette indicating the quantity of antibody. In one embodiment, the antibody is supplied as a dry sterilized lyophilized powder or water free concentrate in a hermetically sealed container and can be reconstituted, e.g., with water or saline to the appropriate concentration for administration to a subject. Preferably, the antibody is supplied as a dry sterile lyophilized powder in a hermetically sealed container at a unit dosage of at least 0.5 mg, at least 1 mg, at least 2 mg, or at least 3 mg, and more preferably at least 5 mg, at least 10 mg, at least 15 mg, at least 25 mg, at least 30 mg, at least 35 mg, at least 45 mg, at least 50 mg, at least 60 mg, or at least 75 mg. The lyophilized antibody can be stored at between 2 and 8° C. in its original container and the antibody can be administered within 12 hours, preferably within 6 hours, within 5 hours, within 3 hours, or within 1 hour after being reconstituted. In an alternative embodiment, a modified antibody is supplied in liquid form in a hermetically sealed container indicating the quantity and concentration of the antibody. Preferably, the liquid form of the antibody is supplied in a hermetically sealed container at least 0.1 mg/ml, at least 0.5 mg/ml, or at least 1 mg/ml, and more preferably at least 2.5 mg/ml, at least 3 mg/ml, at least 5 mg/ml, at least 8 mg/ml, at least 10 mg/ml, at least 15 mg/ml, at least 25 mg/ml, at least 30 mg/ml, or at least 60 mg/ml.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a therapeutic agent (e.g., an antibody of the invention), or a composition of the invention that will be effective in the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof can be determined by standard clinical techniques. For example, the dosage of a therapeutic agent, or a composition comprising an antibody of the invention that will be effective in the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof can be determined by administering the composition to a cotton rat, measuring the RSV titer after challenging the cotton rat with 10⁵ pfu of RSV and comparing the RSV titer to that obtain for a cotton rat not administered the therapeutic agent, or the composition. Accordingly, a dosage that results in a 2 log decrease or a 99% reduction in RSV titer in the cotton rat challenged with 10⁵ pfu of RSV relative to the cotton rat challenged with 10⁵ pfu of RSV but not administered the therapeutic agent, or the composition is the dosage of the composition that can be administered to a human for the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof.

The dosage of a composition which will be effective in the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof can be determined by administering the composition to an animal model (e.g., a cotton rat or monkey) and measuring the serum titer, lung concentration or nasal turbinate and/or nasal secretion concentration of a modified antibody that immunospecifically bind to a RSV antigen. Accordingly, a dosage of an antibody or a composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, and preferably at least 2 μg/ml, at least 5 μg/ml, at least 10 μg/ml, at least 15 μg/ml, at least 20 μg/ml, at least 25 μg/ml, at least 30 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the treating, managing, and/or ameliorating respiratory conditions, including, but not limited to, long term consequences of RSV infection and/or RSV disease, such as, for example, asthma, wheezing, reactive airway disease (RAD), chronic obstructive pulmonary disease (COPD), or a combination thereof. In addition, in vitro assays may optionally be employed to help identify optimal dosage ranges.

The precise dose to be employed in the formulation will also depend on the route of administration, and the seriousness of the RSV URI and/or LRI, and should be decided according to the judgment of the practitioner and each patient's circumstances. Effective doses may be extrapolated from dose-response curves derived from in vitro or animal model (e.g., the cotton rat or Cynomolgous monkey) test systems.

For the antibodies of the invention, the dosage administered to a patient is typically 0.0.25 mg/kg to 100 mg/kg of the patient's body weight. In some embodiments, the dosage administered to the patient is about 3 mg/kg to about 60 mg/kg of the patient's body weight. Preferably, the dosage administered to a patient is between 0.025 mg/kg and 20 mg/kg of the patient's body weight, more preferably 1 mg/kg to 15 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies of the invention may be reduced by enhancing uptake and tissue penetration (e.g., into the nasal passages and/or lung) of the antibodies by modifications such as, for example, lipidation. In a other embodiment, the dosage to be administered to is about 100 mg/kg, about 60 mg/kg, about 50 mg/kg, about 40 mg/kg, about 30 mg/kg, about 15 mg/kg, about 10 mg/kg, about 5 mg/kg, about 3 mg/kg, about 2 mg/kg, about 1 mg/kg, about 0.80 mg/kg, about 0.50 mg/kg, about 0.40 mg/kg, about 0.20 mg/kg, about 0.10 mg/kg, about 0.05 mg/kg, or about 0.025 mg/kg of the patient's body weight.

In a specific embodiment, antibodies of the invention, or compositions comprising antibodies of the invention are administered once a month just prior to (e.g., within three months, within two months, within one month) or during the RSV season. In another embodiment, antibodies of the invention, or compositions comprising modified antibodies of the invention are administered every two months just prior to or during the RSV season. In another embodiment, antibodies of the invention, or compositions comprising antibodies of the invention are administered every three months just prior to or during the RSV season. In another embodiment, antibodies of the invention, or compositions comprising antibodies of the invention are administered once just prior to or during the RSV season. In another embodiment, antibodies of the invention are administered twice, and most preferably once, during a RSV season. In some embodiments, antibodies of the invention are administered just prior to the RSV season and can optionally administered once during the RSV season. In some embodiments, antibodies of the invention, or compositions comprising antibodies of the invention, are administered every 24 hours for at least three days, at least four days, at least five days, at least six days up to one week just prior to or during an RSV season. In specific embodiments, the daily administration of antibodies of the invention, or compositions comprising antibodies of the invention, occur soon after RSV infection is first recognized (i.e., when the patient has nasal congestion and/or other upper respiratory symptoms), but prior to presentation of clinically significant disease in the lungs (i.e., prior to lower respiratory disease manifestation) such that lower respiratory disease is prevented. In another embodiment, modified antibodies of the invention, or compositions comprising modified antibodies of the invention are administered intranasally once a day for about three (3) days while the patient presents with symptoms of RSV URI during the RSV season. Alternatively, in another embodiment, modified antibodies of the invention, or compositions comprising modified antibodies of the invention are administered intranasally once every other day for at least one week while the patient presents with symptoms of RSV URI during the RSV season. In yet another embodiment, modified antibodies of the invention are administered intranasally 12 hours post RSV-infection to a human patient who presents with an RSV viral load of about an M.O.I of 0.1. In yet another embodiment, modified antibodies of the invention are administered intranasally 24 hours post RSV-infection to a human patient who presents with an RSV viral load of about an M.O.I of 0.1. In yet another embodiment, modified antibodies of the invention are administered intranasally 48 hours post RSV-infection to a human patient who presents with an RSV viral load of about an M.O.I of 0.01.

The term “RSV season” refers to the season when RSV infection is most likely to occur. Typically, the RSV season in the northern hemisphere commences in November and lasts through April, but may be extended from August to June in the northern hemisphere, depending upon a region's climate. Preferably, the antibody comprises the VH and VL domain of MEDI-524 comprising a modified IgG (e.g., IgG1) constant domain, or FcRn binding fragment thereof (e.g., the Fc domain or hinge-Fc domain), described herein or an antigen-binding fragment thereof.

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, or approximately 1.5 mg/kg or less of an antibody the invention is administered 5 times, 4 times, 3 times, 2 times or, preferably, 1 time during a RSV season to a subject, preferably a human. In some embodiments, an antibody of the invention is administered about 1-12 times during the RSV season to a subject, wherein the doses may be administered as necessary, e.g., weekly, biweekly, monthly, bimonthly, trimonthly, etc., as determined by a physician. In some embodiments, a lower dose (e.g., 5-15 mg/kg) can be administered more frequently (e.g., 3-6 times) during a RSV season. In other embodiments, a higher dose (e.g., 30-60 mg/kg) can be administered less frequently (e.g., 1-3 times) during a RSV season. However, as will be apparent to those in the art, other dosing amounts and schedules are easily determinable and within the scope of the invention. In other embodiments, an antibody of the invention comprises one or more VH domains or chains and/or one or more VL domains or chains on Table 1, and comprises a modified constant domain described, such as modifications at those residues in the IgG constant domain identified herein.

In one embodiment, approximately 60 mg/kg or less, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered to a patient five times during a RSV season to a subject, preferably a human, intramuscularly or intranasally. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered to a patient three times during a RSV season to a subject, preferably a human, intramuscularly or intranasally. In yet another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention is administered two times and most preferably one time during a RSV season to a subject, preferably a human, intramuscularly or intranasally. In another embodiment, approximately 1 mg/kg or less, approximately 0.1 mg/kg or less, approximately 0.05 mg/kg or less or approximately 0.025 mg/kg of a modified antibody of the invention is administered once a day for at least three days or alternatively, every other day for at least one week during a RSV season to a subject, preferably human, intranasally.

In a specific embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of an antibody the invention in a sustained release formulation is administered to a subject, preferably a human, to prevent, manage, treat and/or ameliorate a RSV infection (e.g., acute RSV disease, or a RSV URI and/or LRI), and/or a symptom or respiratory condition relating thereto (e.g., asthma, wheezing, and/or RAD). In another specific embodiment, an approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less bolus of an antibody the invention not in a sustained release formulation is administered to a subject, preferably a human, to prevent, manage, treat and/or ameliorate a RSV infection (e.g., acute RSV disease, or a RSV URI and/or LRI), and/or a symptom or respiratory condition relating thereto (e.g., asthma, wheezing, and/or RAD), and after a certain period of time, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of the invention in a sustained release is administered to said subject (e.g., intranasally or intramuscularly) two, three or four times (preferably one time) during a RSV season. In accordance with this embodiment, a certain period of time can be 1 to 5 days, a week, two weeks, or a month. In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of a modified antibody of the invention in a sustained release formulation is administered to a subject, preferably a human, intramuscularly or intranasally two, three or four times (preferably one time) during a RSV season to prevent, manage, treat and/or ameliorate a RSV infection (e.g., acute RSV disease, or a RSV URI and/or LRI), and/or a symptom or respiratory condition relating thereto (e.g., asthma, wheezing, and/or RAD).

In another embodiment, approximately 60 mg/kg, approximately 45 mg/kg or less, approximately 30 mg/kg or less, approximately 15 mg/kg or less, approximately 10 mg/kg or less, approximately 5 mg/kg or less, approximately 3 mg/kg or less, approximately 2 mg/kg or less, approximately 1.5 mg/kg or less, approximately 1 mg/kg or less, approximately 0.80 mg/kg or less, approximately 0.50 mg/kg or less, approximately 0.40 mg/kg or less, approximately 0.20 mg/kg or less, approximately 0.10 mg/kg or less, approximately 0.05 mg/kg or less, or approximately 0.025 mg/kg or less of one or more antibodies of the invention is administered intranasally to a subject to prevent, manage, treat and/or ameliorate a RSV infection (e.g., acute RSV disease, or a RSV URI and/or LRI), and/or a symptom or respiratory condition relating thereto (e.g., asthma, wheezing, and/or RAD). In one embodiment, antibodies of the invention are administered intranasally to a subject to treat URI and to prevent lower respiratory tract infection and/or RSV disease.

In certain embodiments, a single dose of a modified antibody of the invention (preferably a MEDI-524 or a modified MEDI-524 antibody, such as MEDI-524-YTE) is administered to a patient, wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, or about 75 mg/kg. In specific embodiments, a single dose of a modified antibody of the invention is administered once per year or once during the course of a RSV season, or once within 3 months, 2 months, or 1 month prior to a RSV season. In some embodiments, a single dose of an antibody of the invention is administered to a patient two, three, four, five, six, seven, eight, nine, ten, eleven, twelve times, thirteen, fourteen, fifteen, sixteen, seventeen, eighteen, nineteen, twenty, twenty-one, twenty-two, twenty-three, twenty-four, twenty five, or twenty six at bi-weekly (e.g., about 14 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical).

In another embodiment, a single dose of an antibody of the invention is administered to patient two, three, four, five, six, seven, eight, nine, ten, eleven, or twelve times at about monthly (e.g., about 30 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each dose monthly dose may or may not be identical).

In one embodiment, a single dose of an antibody of the invention is administered to a patient two, three, four, five, or six times at about bi-monthly (e.g., about 60 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each bi-monthly dose may or may not be identical).

In some embodiments, a single dose of an antibody of the invention is administered to a patient two, three, or four times at about tri-monthly (e.g., about 120 day) intervals over the course of a year (or alternatively over the course of a RSV season), wherein the dose is selected from the group consisting of about 0.025 mg/kg, about 0.05 mg/kg, about 0.10 mg/kg, about 0.20 mg/kg, about 0.40 mg/kg, about 0.50 mg/kg, about 0.80 mg/kg, or about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, or a combination thereof (i.e., each tri-monthly dose may or may not be identical).

In certain embodiments, the route of administration for a dose of an antibody of the invention to a patient is intranasal, intramuscular, intravenous, or a combination thereof, but other routes described herein are also acceptable. Each dose may or may not be administered by an identical route of administration). In some embodiments, an antibody of the invention may be administered via multiple routes of administration simultaneously or subsequently to other doses of the same or a different antibody of the invention.

In certain embodiments, antibodies of the invention are administered therapeutically to a subject (e.g., an infant, an infant born prematurely, an immunocompromised subject, a medical worker, or an elderly subject). Antibodies of the invention can be therapeutically administered to a subject so as to prevent a RSV infection from being transmitted from one individual to another, or to lessen the infection that is transmitted. In some embodiments, the subject has been exposed to (and may or may not be asymptomatic) or is likely to be exposed to another individual having RSV infection (e.g., acute RSV disease, or a RSV URI and/or LRI). For example, said subjects include, but are not limited to, a child in the same school or daycare as another RSV-infected child or other RSV-infected individual, an elderly person in a nursing home as an other RSV-infected individual, or an individual in the same household as a RSV infected child or other RSV-infected individual, medical staff at a hospital working with RSV-infected patients, etc. Preferably, the antibody administered therapeutically to the subject is administered intranasally, but other routes of administration described herein are acceptable. In some embodiments, the antibody of the invention is administered (e.g., intranasally) at a dose of about 0.025 mg/kg, about 0.05 mg/kg, about 0.10 mg/kg, about 0.20 mg/kg, about 0.40 mg/kg, about 0.50 mg/kg, about 0.80 mg/kg, about 1 mg/kg, about 2 mg/kg, about 3 mg/kg, about 5 mg/kg, about 10 mg/kg, about 15 mg/kg, about 30 mg/kg, about 40 mg/kg, or about 50 mg/kg. Lower dosages and less frequent administration is preferred, for example, intranasal administration (or other route) once every 2-4 hours, 4-6 hours, 6-8 hours, 8-10 hours, 10-12 hours, 12-14 hours, 14-16 hours, 16-18 hours, 18-20 hours, 20-22 hours, 22-24 hours (preferably once or twice per day) for about 3 days, about 5 days or about 7 days or as otherwise needed after potential or actual exposure to the RSV-infected individual. Any antibody of the invention described herein may be used, and in certain embodiments the antibody comprises a modified IgG (e.g., IgG1) constant domain, or FcRn binding fragment thereof (e.g., the Fc domain or hinge-Fc domain).

5.4 Diagnostic Uses of Antibodies

Labeled antibodies of the invention (modified) and derivatives and analogs thereof, which immunospecifically bind to a RSV antigen can be used for diagnostic purposes to detect, diagnose, or monitor a RSV URI and/or LRI. The invention provides methods for the detection of a RSV infection (e.g., a RSV URI and/or LRI), or a symptom or respiratory condition relating thereto (including, but not limited to, asthma, wheezing, RAD, or a combination thereof) comprising: (a) assaying the expression of a RSV antigen in cells or a tissue sample of a subject using one or more antibodies of the invention that immunospecifically bind to the RSV antigen; and (b) comparing the level of the RSV antigen with a control level, e.g., levels in normal tissue samples not infected with RSV, whereby an increase in the assayed level of RSV antigen compared to the control level of the RSV antigen is indicative of a RSV infection (e.g., a RSV URI and/or LRI), or a symptom or respiratory condition relating thereto (including, but not limited to, asthma, wheezing, RAD, or a combination thereof).

The invention provides a diagnostic assay for diagnosing a RSV infection (e.g., a RSV URI and/or LRI), or a symptom or respiratory condition relating thereto (including, but not limited to, asthma, wheezing, RAD, or a combination thereof) comprising: (a) assaying for the level of a RSV antigen in cells or a tissue sample of an individual using one or more antibodies of the invention that immunospecifically bind to a RSV antigen; and (b) comparing the level of the RSV antigen with a control level, e.g., levels in normal tissue samples not infected with RSV, whereby an increase in the assayed RSV antigen level compared to the control level of the RSV antigen is indicative of a RSV infection (e.g., a RSV URI and/or LRI), or a symptom or respiratory condition relating thereto (including, but not limited to, asthma, wheezing, RAD, or a combination thereof). A more definitive diagnosis of a RSV infection (e.g., a RSV URI and/or LRI), or a symptom or respiratory condition relating thereto (including, but not limited to, asthma, wheezing, RAD, or a combination thereof) may allow health professionals to employ preventative measures or aggressive treatment earlier thereby preventing the development or further progression of the RSV infection.

5.5 Biological Activity and Assays for Modified Antibodies

Antibodies of the present invention may be characterized in a variety of ways. In particular, antibodies of the invention may be assayed for the ability to immunospecifically bind to a RSV antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). Antibodies that have been identified to immunospecifically bind to a RSV antigen (e.g., a RSV F antigen) can then be assayed for their specificity and affinity for a RSV antigen.

The modified antibodies of the invention may be assayed for immunospecific binding to a RSV antigen and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the present invention for a RSV antigen and the binding off-rates can be determined from the data by scatchard plot analysis. Competition with a second antibody can also be determined using radioimmunoassays. In this case, a RSV antigen is incubated with an antibody of the present invention conjugated to a labeled compound (e.g., ³H or ¹²⁵I) in the presence of increasing amounts of an unlabeled second antibody.

In a other embodiment, BIAcore kinetic analysis is used to determine the binding on and off rates of antibodies to a RSV antigen. BIAcore kinetic analysis comprises analyzing the binding and dissociation of a RSV antigen from chips with immobilized antibodies on their surface.

The antibodies of the invention can also be assayed for their ability to inhibit the binding of RSV to its host cell receptor using techniques known to those of skill in the art. For example, cells expressing the receptor for RSV can be contacted with RSV in the presence or absence of an antibody and the ability of the antibody to inhibit RSV's binding can measured by, for example, flow cytometry or a scintillation assay. RSV (e.g., a RSV antigen such as F glycoprotein or G glycoprotein) or the antibody can be labeled with a detectable compound such as a radioactive label (e.g., 32P, 35S, and 125I) or a fluorescent label (e.g., fluorescein isothiocyanate, rhodamine, phycoerythrin, phycocyanin, allophycocyanin, o-phthaldehyde and fluorescamine) to enable detection of an interaction between RSV and its host cell receptor. Alternatively, the ability of antibodies to inhibit RSV from binding to its receptor can be determined in cell-free assays. For example, RSV or a RSV antigen such as G glycoprotein can be contacted with an antibody and the ability of the antibody to inhibit RSV or the RSV antigen from binding to its host cell receptor can be determined. Preferably, the antibody is immobilized on a solid support and RSV or a RSV antigen is labeled with a detectable compound. Alternatively, RSV or a RSV antigen is immobilized on a solid support and the antibody is labeled with a detectable compound. RSV or a RSV antigen may be partially or completely purified (e.g., partially or completely free of other polypeptides) or part of a cell lysate. Further, a RSV antigen may be a fusion protein comprising the RSV antigen and a domain such as glutathionine S transferase. Alternatively, a RSV antigen can be biotinylated using techniques well known to those of skill in the art (e.g., biotinylation kit, Pierce Chemicals; Rockford, Ill.).

The antibodies of the invention can also be assayed for their ability to inhibit or downregulate RSV replication using techniques known to those of skill in the art. For example, RSV replication can be assayed by a plaque assay such as described, e.g., by Johnson et al., 1997, Journal of Infectious Diseases 176:1215-1224. The modified antibodies of the invention can also be assayed for their ability to inhibit or down-regulate the expression of RSV polypeptides. Techniques known to those of skill in the art, including, but not limited to, Western blot analysis, Northern blot analysis, and RT-PCR can be used to measure the expression of RSV polypeptides. Further, the antibodies of the invention can be assayed for their ability to prevent the formation of syncytia.

The ability of the antibodies described herein or fragments thereof to block RSV-induced fusion after viral attachment to the cells is determined in a fusion inhibition assay. This assay is identical to the microneutralization assay, except that the cells were infected with RSV (Long) for four hours prior to addition of antibody (Taylor et al, 1992, J. Gen. Virol. 73:2217-2223).

Modified antibodies or compositions of the invention can be tested in vitro and in vivo for the ability to induce or inhibit the expression of cytokines by an RSV-infected tissue/cell, such as IFN-α, IFN-β, IFN-γ, IL-2, IL-3, IL-4, IL-5, IL-6, IL-7, IL-8, IL-9, IL-10, IL-12 and IL-15. Techniques known to those of skill in the art can be used to measure the level of expression of cytokines. For example, the level of expression of cytokines can be measured by analyzing the level of RNA of cytokines by, for example, RT-PCR and Northern blot analysis, and by analyzing the level of cytokines by, for example, immunoprecipitation followed by western blot analysis and ELISA. The results of the modified antibody of the invention can be compared to the same antibody without the modifications, as described herein. The difference in cytokine response may be quantified by a relative percent: about 5% difference, about 10% difference, about 15% difference, about 20% difference, about 25% difference, about 30% difference, about 35% difference, about 40% difference, about 45% difference, about 50% difference, about 55% difference, about 60% difference, about 65% difference, about 70% difference, about 75% difference, about 80% difference, about 85% difference, about 90% difference, about 95% difference, about 100% difference, and so on. It is envisioned that the modified antibodies of the invention will, in one embodiment, inhibit the expression of cytokines by the RSV-infected tissues/cells (see Examples).

Alternatively, the level of expression of cytokines can be measured by analyzing the serum level of cytokines in a human patient. Such techniques as well known to those skilled in the art. For example, whole blood samples can be collected from treated patients and placed into tubes. The blood samples can be incubated at 37° C. in a 5% CO₂ saturated, humidified incubator. The blood samples can be spun, and the supernatant separated, flash-frozen, and stored at −20° C. Cytokines can then be assayed by any standard, conventional bioassay well known to those skilled in the art. For example, cytokine levels, such as, for example, TNF-alpha can be measured using IRMA kits (Medgenix, Brussels, Belgium). Alternatively, RIA assays can be used with specific commercially available antibodies against specific cytokines to sample whole blood supernatants.

Antibodies or compositions of the invention can be tested in vitro and in vivo for the ability to induce or inhibit the expression of chemokines by affector and memory lymphocytes in response to RSV-infected tissues/cells, such as CC, CXC or C chemokines, well known to those skilled in the art. Techniques known to those of skill in the art can be used to measure the level of expression of chemokines. For example, the level of expression of cytokines can be measured by analyzing the level of RNA of chemokines by, for example, RT-PCR and Northern blot analysis, and by analyzing the level of chemokines by, for example, immunoprecipitation followed by western blot analysis and ELISA. The results of the modified antibody of the invention can be compared to the same antibody without the modifications, as described herein. The difference in chemokine response may be quantified by a relative percent: about 5% difference, about 10% difference, about 15% difference, about 20% difference, about 25% difference, about 30% difference, about 35% difference, about 40% difference, about 45% difference, about 50% difference, about 55% difference, about 60% difference, about 65% difference, about 70% difference, about 75% difference, about 80% difference, about 85% difference, about 90% difference, about 95% difference, about 100% difference, and so on. It is envisioned that the modified antibodies of the invention will, in one embodiment, inhibit the expression of chemokines by the affector and memory lymphocytes in response to RSV-infected tissues/cells.

Alternatively, the level of expression of chemokines can be measured by analyzing the serum level of chemokines in a human patient. Such techniques as well known to those skilled in the art. For example, an ELISA can be employed after obtaining whole blood sample supernatants, as described above.

Antibodies or compositions of the invention can be tested in vitro and in vivo for their ability to modulate the biological activity of immune cells, preferably human immune cells (e.g., T-cells, B-cells, and Natural Killer cells). The ability of an antibody or composition of the invention to modulate the biological activity of immune cells can be assessed by detecting the expression of antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by ³H thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipition reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs).

Antibodies or compositions of the invention can also be tested for their ability to inhibit viral replication or reduce viral load in in vitro, ex vivo and in vivo assays. For example, neutralization of the antibodies described herein can be determined by a microneutralization assay. This microneutralization assay is a modification of the procedures described by Anderson et al. (1985, J. Clin. Microbiol. 22:1050-1052, the disclosure of which is hereby incorporated by reference in its entirety). The procedures are also described in Johnson et al., 1999, J. Infectious Diseases 180:35-40, the disclosure of which is hereby incorporated by reference in its entirety. Briefly, antibody dilutions are made in triplicate using a 96-well plate. Virus is incubated with serial dilutions of the antibodies of the invention to be tested for 2 hours at 37 C in the wells of a 96-well plate. RSV susceptible HEp-2 cells (2.5×10⁴) are added to each well and can be cultured for 5 days at 37 C in 5% CO₂. After 5 days, the medium was aspirated and cells were washed and fixed to the plates with 80% methanol and 20% PBS. RSV replication can be determined by F protein expression. Fixed cells can be incubated with a biotin-conjugated anti-F protein monoclonal antibody (pan F protein, C-site-specific MAb 133-1H) and detected by horseradish peroxidase conjugated avidin and turnover of substrate TMB (thionitrobenzoic acid), measured at 450 nm. The neutralizing titer can be expressed as the antibody concentration that caused at least 50% reduction in absorbency at 450 nm (the OD₄₅₀) from virus-only control cells.

Antibodies or compositions of the invention can also be tested for their ability to decrease the time course of a RSV infection (e.g., a RSV URI and/or LRI), or a symptom or respiratory condition relating thereto (including, but not limited to, asthma, wheezing, RAD, or a combination thereof). Antibodies or compositions of the invention can also be tested for their ability to increase the survival period of humans suffering from a RSV infection (preferably, a RSV URI and/or LRI) by at least 25%, at least 50%, at least 60%, at least 75%, at least 85%, at least 95%, or at least 99%. Further, antibodies or compositions of the invention can be tested for their ability reduce the hospitalization period of humans suffering from a RSV infection (preferably, a RSV URI and/or LRI) by at least 60%, at least 75%, at least 85%, at least 95%, or at least 99% as compared to placebo or a human who did not receive a therapeutic administration of the antibodies of the invention. Techniques known to those of skill in the art can be used to analyze the function of the antibodies or compositions of the invention in vivo.

The binding ability of IgGs and molecules comprising an IgG constant domain of FcRn fragment thereof to FcRn can be characterized by various in vitro assays. PCT publication WO 97/34631 by Ward discloses various methods in detail and is incorporated herein in its entirety by reference.

For example, in order to compare the ability of a modified antibody of the invention or fragments thereof to bind to FcRn with that of the unmodified or wild type IgG, the modified IgG or fragments thereof and the unmodified or wild type IgG can be radio-labeled and reacted with FcRn-expressing cells in vitro. The radioactivity of the cell-bound fractions can be then counted and compared. The cells expressing FcRn to be used for this assay are preferably endothelial cell lines including mouse pulmonary capillary endothelial cells (B10, D2.PCE) derived from lungs of B10.DBA/2 mice and SV40 transformed endothelial cells (SVEC) (Kim et al., J. Immunol., 40:457-465, 1994) derived from C3H/HeJ mice. However, other types of cells, such as intestinal brush borders isolated from 10- to 14-day old suckling mice, which express sufficient number of FcRn can be also used. Alternatively, mammalian cells which express recombinant FcRn of a species of choice can be also utilized. After counting the radioactivity of the bound fraction of modified IgG or that of the unmodified or wild type, the bound molecules can be then extracted with the detergent, and the percent release per unit number of cells can be calculated and compared.

Affinity of modified IgGs for FcRn can be measured by surface plasmon resonance (SPR) measurement using, for example, a BIAcore 2000 (BIAcore Inc.) as described previously (Popov et al., Mol. Immunol., 33:493-502, 1996; Karlsson et al., J. Immunol. Methods, 145:229-240, 1991, both of which are incorporated by reference in their entireties). In this method, FcRn molecules are coupled to a BIAcore sensor chip (e.g., CM5 chip by Pharmacia) and the binding of modified IgG to the immobilized FcRn is measured at a certain flow rate to obtain sensorgrams using BIA evaluation 2.1 software, based on which on- and off-rates of the modified IgG, constant domains, or fragments thereof, to FcRn can be calculated.

Relative affinities of modified IgGs or fragments thereof, and the unmodified or wild type IgG for FcRn can be also measured by a simple competition binding assay. Unlabeled modified IgG or unmodified or wild type IgG is added in different amounts to the wells of a 96-well plate in which FcRn is immobilize. A constant amount of radio-labeled unmodified or wild type IgG is then added to each well. Percent radioactivity of the bound fraction is plotted against the amount of unlabeled modified IgG or unmodified or wild type IgG and the relative affinity of the modified hinge-Fc can be calculated from the slope of the curve.

Furthermore, affinities of modified IgGs or fragments thereof, and the wild type IgG for FcRn can be also measured by a saturation study and the Scatchard analysis.

Transfer of modified IgG or fragments thereof across the cell by FcRn can be measured by in vitro transfer assay using radiolabeled IgG or fragments thereof and FcRn-expressing cells and comparing the radioactivity of the one side of the cell monolayer with that of the other side. Alternatively, such transfer can be measured in vivo by feeding 10- to 14-day old suckling mice with radiolabeled, modified IgG and periodically counting the radioactivity in blood samples which indicates the transfer of the IgG through the intestine to the circulation (or any other target tissue, e.g., the lungs). To test the dose-dependent inhibition of the IgG transfer through the gut, a mixture of radiolabeled and unlabeled IgG at certain ratio is given to the mice and the radioactivity of the plasma can be periodically measured (Kim et al., Eur. J. Immunol., 24:2429-2434, 1994).

The half-life of modified IgG or fragments thereof can be measured by pharmacokinetic studies according to the method described by Kim et al. (Eur. J. of Immuno. 24:542, 1994), which is incorporated by reference herein in its entirety. According to this method, radiolabeled modified IgG or fragments thereof is injected intravenously into mice and its plasma concentration is periodically measured as a function of time, for example, at 3 minutes to 72 hours after the injection. The clearance curve thus obtained should be biphasic, that is, α-phase and β-phase. For the determination of the in vivo half-life of the modified IgGs or fragments thereof, the clearance rate in β-phase is calculated and compared with that of the unmodified or wild type IgG.

The effector functions of a modified antibody of the invention can be measured by an ADCC assay (see Examples). Chromium assays are well-known in the art (see, for example, Brunner, K. T. et al., (1968) Quantitative Assay of the Lytic Action of Immune Lymphoid Cells on Cr-labelled Allogenic Target Cells in-vitro; Inhibition by Iso-antibody and by Drugs, Immunology 14, 181). More recently, LDH cytotoxicity assays are being used. The assay is based on measurement of activity of lactate dehydrogenase (LDH) which is a stable enzyme normally found in the cytosol of all cells but rapidly releases into the supernatant upon damage of plasma membrane. Results can be analyzed by spectrophotometry at 500 nm. Such assays are available commercially as kits, therefore are readily available to those of skill in the art.

5.6 Methods of Producing Antibodies

Antibodies of the invention that immunospecifically bind to an antigen can be produced by any method known in the art for the synthesis of antibodies, in particular, by chemical synthesis or preferably, by recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Antibody fragments which recognize specific RSV antigens (preferably, RSV F antigen) may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

For example, antibodies can also be generated using various phage display methods. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. Examples of VL and VH constant domains that can be used in certain embodiments of the invention include, but are not limited to, C-kappa and C-gamma-1 (nG1m) described in Johnson et al. (1997) J. Infect. Dis. 176, 1215-1224 and those described in U.S. Pat. No. 5,824,307. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibodies can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated by reference herein in its entirety. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Reichmann et al., 1988, Nature 332:323, which are incorporated herein by reference in their entireties.)

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to a RSV antigen (e.g., a RSV F antigen) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

5.6.1 Polynucleotides Encoding an Antibody

The invention provides polynucleotides comprising a nucleotide sequence encoding an antibody (modified) of the invention that immunospecifically binds to a RSV antigen (e.g., RSV F antigen).

The polynucleotides may be obtained, and the nucleotide sequence of the polynucleotides determined, by any method known in the art. Since the amino acid sequences of AFFF, P12f2, P12f4, P11d4, A1e9, A12a6, A13c4, A17d4, A4B4, A8c7, 1X-493L1FR, H3-3F4, M3H9, Y10H6, DG, AFFF(1), 6H8, L1-7E5, L2-15B10, A13a11, A1h5, A4B4(1), MEDI-524, A4B4-F52S, A17d4(1), A3e2, A14a4, A16b4, A17b5, A17f5, or A17h4 are known (see, e.g., Table 1), nucleotide sequences encoding these antibodies and modified versions of these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, fragments, or variants thereof, annealing and ligating of those oligonucleotides, and then amplification of the ligated oligonucleotides by PCR.

Alternatively, a polynucleotide encoding an antibody of the invention may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

5.6.2 Mutagenesis

Once the nucleotide sequence of the antibody is determined the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions. In certain embodiments, amino acid substitutions, deletions and/or insertions are introduced into the epitope-binding domain regions of the antibodies and/or into the hinge-Fc regions of the antibodies which are involved in the interaction with the FcRn.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278:457-479 for a listing of human framework regions). Preferably, the polynucleotide sequence generated by the combination of the framework regions and CDRs encodes an antibody that immunospecifically binds to a particular antigen, such as the RSV F antigen. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Mutagenesis may be performed in accordance with any of the techniques known in the art including, but not limited to, synthesizing an oligonucleotide having one or more modifications within the sequence of the constant domain of an antibody or a fragment thereof (e.g., the CH2 or CH3 domain) to be modified. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generated a library of mutants.

The technique of site-specific mutagenesis is well known in the art, as exemplified by various publications (see, e.g., Kunkel et al., Methods Enzymol., 154:367-82, 1987, which is hereby incorporated by reference in its entirety). In general, site-directed mutagenesis is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

Alternatively, the use of PCR™ with commercially available thermostable enzymes such as Taq DNA polymerase may be used to incorporate a mutagenic oligonucleotide primer into an amplified DNA fragment that can then be cloned into an appropriate cloning or expression vector. See, e.g., Tomic et al., Nucleic Acids Res., 18(6):1656, 1987, and Upender et al., Biotechniques, 18(1):29-30, 32, 1995, for PCR™-mediated mutagenesis procedures, which are hereby incorporated in their entireties. PCR™ employing a thermostable ligase in addition to a thermostable polymerase may also be used to incorporate a phosphorylated mutagenic oligonucleotide into an amplified DNA fragment that may then be cloned into an appropriate cloning or expression vector (see e.g., Michael, Biotechniques, 16(3):410-2, 1994, which is hereby incorporated by reference in its entirety).

Other methods known to those of skill in art of producing sequence variants of the Fc domain of an antibody or a fragment thereof can be used. For example, recombinant vectors encoding the amino acid sequence of the constant domain of an antibody or a fragment thereof may be treated with mutagenic agents, such as hydroxylamine, to obtain sequence variants.

5.6.3 Panning

Vectors, in particular, phage, expressing constant domains or fragments thereof having one or more modifications in amino acid residues can be screened to identify constant domains or fragments thereof having increased or decreased affinity for FcRn. Immunoassays which can be used to analyze binding of the constant domain or fragment thereof having one or more modifications in amino acid residues to the FcRn include, but are not limited to, radioimmunoassays, ELISA (enzyme linked immunosorbent assay), “sandwich” immunoassays, and fluorescent immunoassays. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly herein below (but are not intended by way of limitation). BIAcore kinetic analysis can also be used to determine the binding on and off rates of a constant domain or a fragment thereof having one or more modifications in amino acid residues to the FcRn. BIAcore kinetic analysis comprises analyzing the binding and dissociation of a constant domain or a fragment thereof having one or more modifications in amino acid residues from chips with immobilized FcRn on their surface.

5.6.4 Sequencing

Any of a variety of sequencing reactions known in the art can be used to directly sequence the nucleotide sequence encoding, e.g., variable regions and/or constant domains or fragments thereof having one or more amino acid Fc domain modifications. Examples of sequencing reactions include those based on techniques developed by Maxim and Gilbert (Proc. Natl. Acad. Sci. USA, 74:560, 1977) or Sanger (Proc. Natl. Acad. Sci. USA, 74:5463, 1977). It is also contemplated that any of a variety of automated sequencing procedures can be utilized (Bio/Techniques, 19:448, 1995), including sequencing by mass spectrometry (see, e.g., PCT Publication No. WO 94/16101, Cohen et al., Adv. Chromatogr., 36:127-162, 1996, and Griffin et al., Appl. Biochem. Biotechnol., 38:147-159, 1993).

5.6.5 Recombinant Expression of an Antibody

Recombinant expression of an antibody of the invention (e.g., a heavy or light chain of an antibody of the invention or a single chain antibody of the invention) that immunospecifically binds to a RSV antigen (e.g., RSV F antigen) requires construction of an expression vector containing a polynucleotide that encodes the antibody. Once a polynucleotide encoding an antibody molecule, heavy or light chain of an antibody, or fragment thereof (preferably, but not necessarily, containing the heavy and/or light chain variable domain) of the invention has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In other embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention which immunospecifically bind to a RSV antigen (preferably, RSV F antigen) is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art to facilitate purification.

### 6. EXAMPLES

### Example 1

### MEDI-524 Treatment Modulates RSV-Induced Cytokine Response

MEDI-524 was added to RSV-infected epithelial cells post-infection to see if administration of the antibody could modulate cytokine release from the RSV infected cells. Two time points of infection were performed, one at 1 hour post-infection, the other at 12 hours post infection.

12 hour time point: 2-12 well plates were seeded with HEp-2 (at passage 9) cells at 5×10⁵ cells/well in 2 mls and allowed to culture for approximately one day to confluency. Confluent Hep-2 cells were infected with RSV A virus (WVB032302) at a MOI=1. After 12 hrs of infection, either control antibody MEDI-507 (20 ug/ml) or MEDI-524 (52405G-0964) (20 ug/ml) were added to appropriate wells at 12 hours post-infection. The cells were incubated for an additional 6 and 24 hours at 37° C./5% CO₂. Supernatants were collected either at the 6 or 24 hours time points by spinning at 1500 rpm for 5 mins) and stored at −80° C. until ready to assay.

1 hour time point: 6-12 well plates were seeded with HEp-2 P10 cells at 5×10⁵ cells/well in 2 mls and allowed to culture for approximately one day to confluency. Confluent Hep-2 cells were infected with RSV A virus (WVB032302) at a MOI=0.5 or 1.0 or 5.0. After 1 hr of infection, the inoculum was removed and 1 ml of fresh media with 10 ug/ml of control antibody MEDI-507 (10.1 mg/ml stock), 10 ug/ml of MEDI-524 (52405G-0336, 10.2 mg/ml stock) or 10 ug/ml of MEDI-524 Fab 2′ (KS011107, 2.75 mg/ml stock) was added to the infected cells. The cells were incubated for an additional 6 and 24 hours at 37° C./5% CO₂ Supernatants were collected either at the 6 or 24 hours time points by spinning at 1500 rpm for 5 mins) and stored at −80° C. until ready to assay.

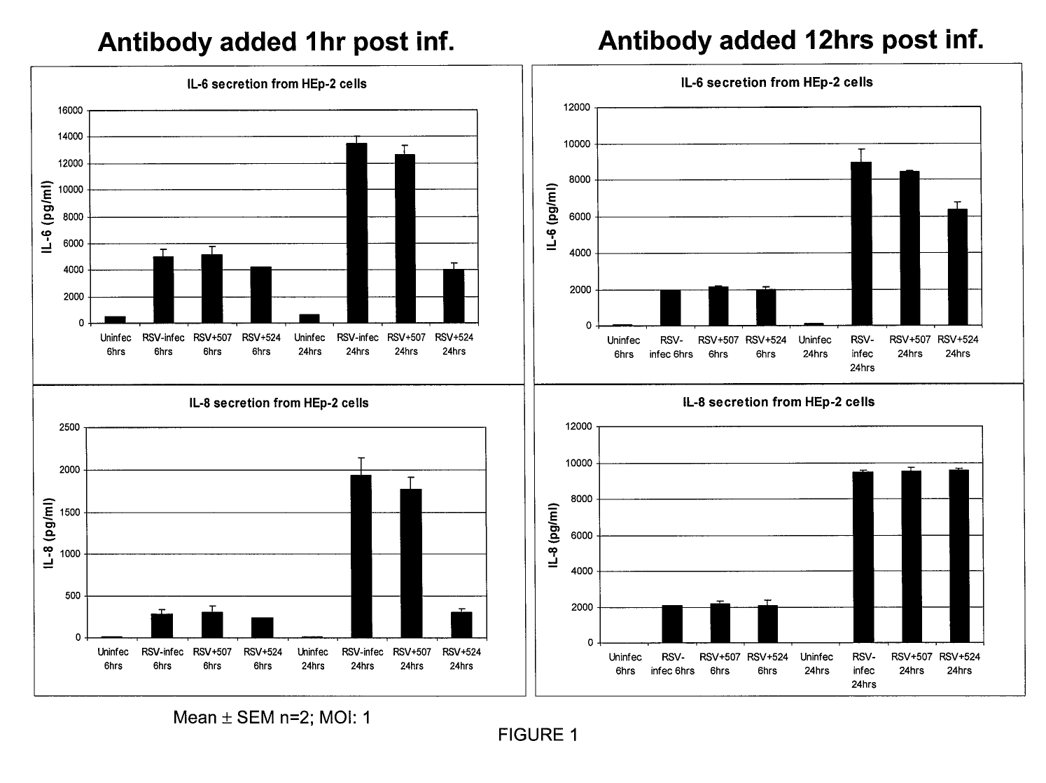

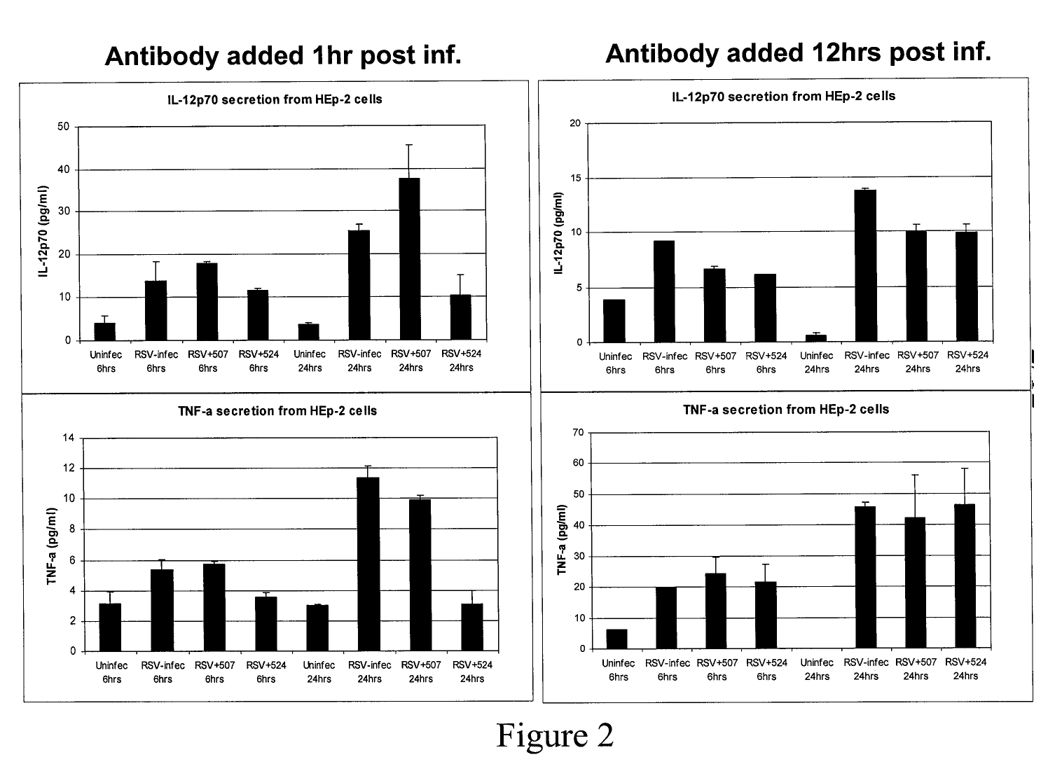

The cytokine assay was performed on the collected supernatants described above using MesoScale Discovery® multiplex kits—the MS6000 Human Proinflammatory-7 Tissue culture kit (Cat # K11008B) and the MS6000 Human Chemokine-9 Tissue culture kit (Cat # K11001B) to assay for IL-6, IL-8, IL-12p70 and TNF-alpha. The results are shown in FIGS. 1 and 2. This experiment shows that earlier therapeutic administration of MEDI-524 at 1 hour, as opposed to 12 hours, and allowing MEDI-524 to incubate with infected cells for 6 hours, as opposed to 24 hours, can decrease cytokine release of RSV-infected cells.

### Example 2

### MEDI-524 Mediated THP-1 Activation

Experiments were performed to determine if MEDI-524 treatment can modulate the chemokine response of activated THP-1 cells responding to RSV-infected cells.

4-12 well plates were seeded with HEp-2 cells (at passage 8) at 5×10⁵ cells/well in 2 mls. THP-1 cells at passage 14 (3×10⁵ cells/ml, 15 mls) and at passage 27 (3.0×10⁵ cells/ml, 15 mls) were activated with IFN-γ (500 U/ml final conc., 15 ul for 15 mls) for 48 hrs.

Approximately 36 hours of culturing, the confluent HEp-2 cells in 12 well plates were infected with RSV A (8×10⁶ pfu/ml) at MOI=1. After 12-15 hours, the infection media was aspirated and rinsed once with FACS buffer (1×PBS with 2% FBS). Control antibody MEDI-507, or MEDI-524 (52405G-0336, 10.2 mg/ml), or MEDI-524 Fab′2 (KS011107, 2.75 mg/ml) were diluted in FACS buffer to a final concentration of 20 ug/ml and added to appropriate wells. After 15 minutes of incubation at room temperature, the antibody-containing FACS buffer was aspirated and rinsed once with fresh THP-1 media.

48 hr-activated THP-1 cells were spun down and resuspended in fresh THP-1 media (to remove any excess IFN-γ). 1 ml of THP-1 cells (in THP-1 media) was added to HEp-2 cells in appropriate wells and incubated for 6 and 24 hrs. The ratio of RSV-infected Hep-2 cells to THP-1 activate cells approximated 2:1. After 6 and 24 hrs of co-culture, supernatant was collected, spun down (1500 rpm, 5 mins) and stored at −80° C. until ready to assay.

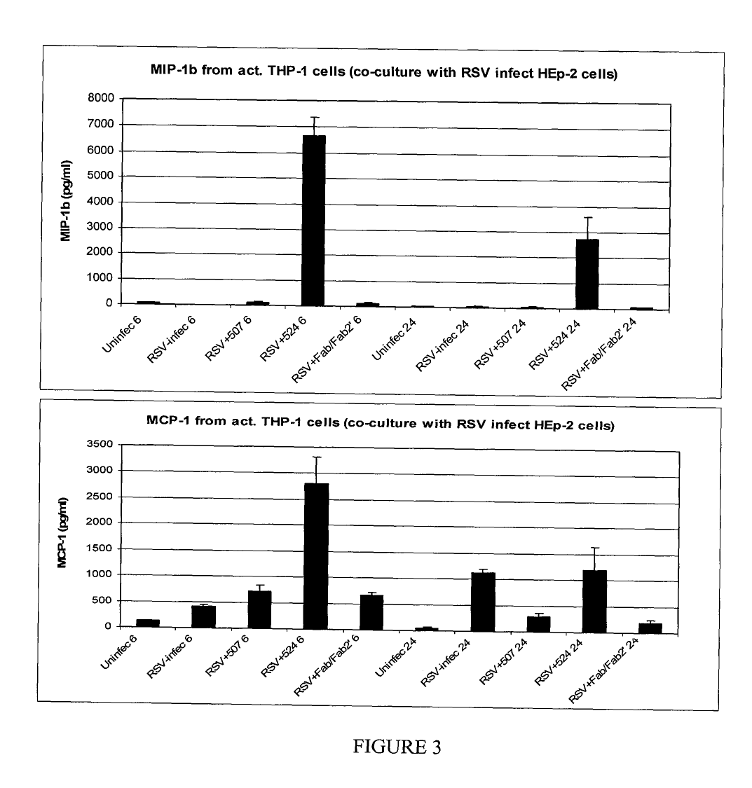

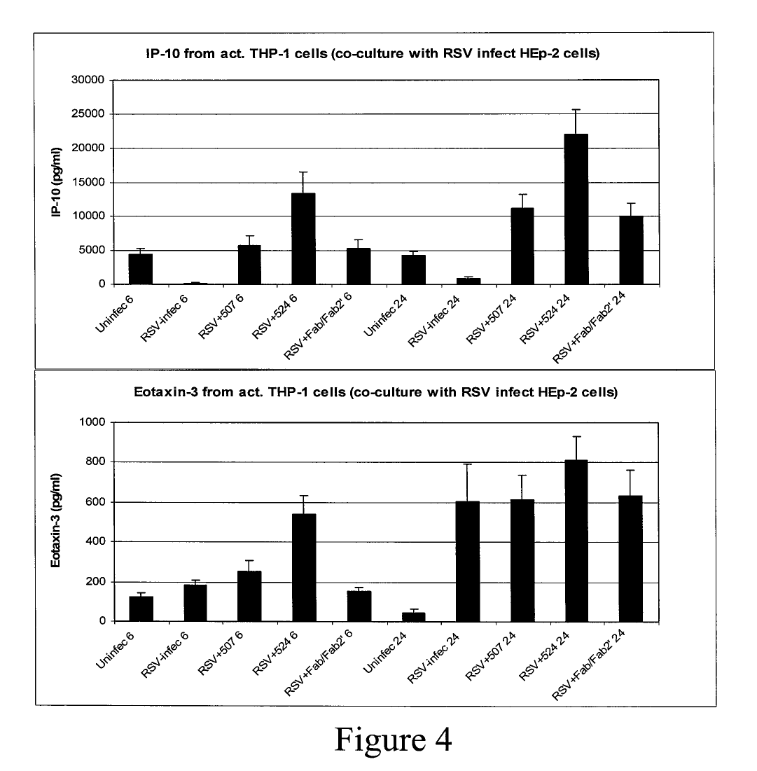

The cytokine assay was performed on the collected supernatants described above using MesoScale Discovery® multiplex kits—the MS6000 Human Proinflammatory-7 Tissue culture kit (Cad # K11008B) and the MS6000 Human Chemokine-9 Tissue culture kit (Cat # K11001B) to assay for chemokine release. Only MIP-1b, MCP-1, IP-10 and eotaxin-3 were measured to be induced. The results are shown in FIGS. 3 and 4. This experiment shows that treatment with MEDI-524 can induce MIP-1b, MCP-1, IP-10 and eotaxin-3 release from activated THP-1 monocytes, but apparently not others.

### Example 3

### MEDI-524 Mediated THP-1 Phagocytosis

Experiments were performed to determine if MEDI-524 treatment can mediate monocyte phagocytosis of RSV-infected cells.

Staining of HEp-2 cells with lipophilic dye: HEp-2 cells at passage 5 were counted and resuspended at 1×10⁶ cells/ml in HBSS in 50 ml conical tube. Next, 2.5 ul of blue dye (Vybrant® DiD cell labeling solution, #V22887, Invitrogen®) was added per ml of HBSS-cell suspension. The cells were incubated at 37° C. for 20 mins and inverted in the 50 ml tube 3 times every 5 mins. Washed the cells 4 times at 1700 rpm for 5 mins with HBSS. The cells were resuspended in complete media and plated in 12 well plates at 5×10⁵ cells/well in a volume of 2 mls (cells became confluent in 48 hrs).

Activation of THP-1 cells: THP-1 cells at passage 19 at 3×10⁵ cells/ml in 12 mls were activated with 500 U/ml IFN-γ (12 ul for 12 mls of THP-1 cells) and incubated at 37° C. for 48 hrs.

Infection of HEp-2 cells with RSV: Confluent HEp-2 cells were infected with RSV A (WVB032302) at MOI 1 for 20 hrs. Afterwards, media was aspirated from the RSV-infected HEp-2 plates. 1 ml of cell dissociation buffer was added to each plate well and incubated at 37° C. for 15 mins. HEp-2 cells were dissociated with 1000 ul pipette tips and transferred to flow tubes. 2 ml of FACS wash buffer was added to each tube and washed at 1500 rpm for 5 mins. HEp-2 cells were resuspended in 100 ul of FACS buffer wash and control antibody MEDI-507 (20 ug/ml), MEDI-524 (20 ug/ml) and MEDI-524 FAB′2 (20 ug/ml) were added to cell suspension and incubated for 20 mins at RT. HEp-2 cells were washed in 2 ml of FACS wash at 1500 rpm/5 mins/4° C. HEp-2 cells were resuspended in 100 ul of THP-1 media. Activated THP-1 cells (3×10⁵ cells/ml in 12 mls) were spun down and resuspended in 12 mls of fresh THP-1 media to remove any excess IFN-γ. 1 ml of THP-1 cells were added to 12-well plate to which the differentially treated HEp-2 cells were added and incubated at 37° C. for 16 hrs. After 16 hrs, cells were aliquoted in flow tubes (described below).

Cells were washed 1× with FACS wash and resuspended in 100 ul of FACS wash. Appropriate tubes were stained with 8 ul of HLADR-PE (555812, BD Biosciences®) for 15 mins, RT in the dark. Cells were washed 1× with FACS wash and fixed with 1% formaldehyde for 15 mins. Fixative was washed away with FACS wash and cells were resuspended in 200 ul of FACS wash and transferred to 96-well NUNC plates to be run on LSRII (green).

### Flow Tubes:

1) Unstained THP-1+unstained HEp-2

2) Unstained HEp-2+THP-1+HLADR-PE

3) Stained HEp-2+unstained THP-1

4) Uninfected stained HEp-2+THP-1+HLADR-PE

5) RSV-infected stained HEp-2+THP-1+HLADR-PE

6) RSV-infected stained HEp-2+Medi507+THP-1+HLADR-PE

7) RSV-infected stained HEp-2+Medi524+THP-1+HLADR-PE

8) RSV-infected stained HEp-2+Medi524+THP-1+HLADR-PE

9) RSV-infected stained HEp-2+Numax FAB′2+THP-1+HLADR-PE

10) RSV-infected stained HEp-2+Numax FAB′2+THP-1+HLADR-PE

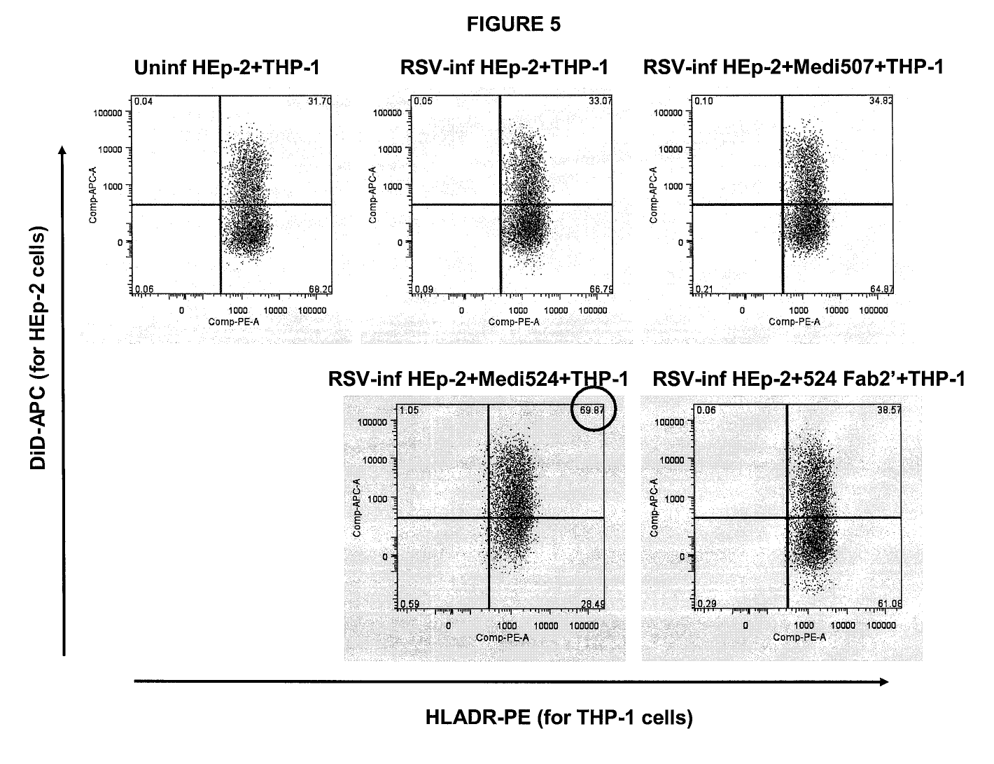

Tube numbers 7-8 and 9-10 are duplicate wells. All THP-1 cells were IFN-γ activated. The results are shown in FIG. 5. FIG. 5 shows that treatment with MEDI-524 can mediate THP-1 monocyte phagocytosis of RSV-infected cells (see RSV-inf Hep-2+MEDI-524+THP-1 panel).

### Example 4

### ADCC Effector Functions

Experiments were performed to determine whether antibody dependent cell-mediated cytotoxicity (ADCC) played a role in RSV treatment with either MEDI-524 or MEDI-524 3M.

Seeded HEp-2 cells at passage 11 in a T75 tissue culture flask at 3.5×10⁶ cells in 20 mls and cultured to confluency. Approximately 36 hours later, confluent HEp-2 cells were infected with RSV A at a MOI=1.0.

Target cells: After 12 hrs of infection, infected HEp-2 cells were dissociated and resuspended in RPMI 1640 (phenol red free) media with 5% FBS (RP-5) at a concentration of 4×10⁵ cells/ml.

Effector cells: NK cells at passage 31 were suspended in RP-5 media at a concentration of 10×10⁵ cells/ml.

Antibodies: MEDI-524 (52405G-0336), MEDI-524-3M (having the amino acid mutations 239D, 330L, 332E as in Kabat numbering), and control antibody R347 were diluted in RP-5 in a concentration range from 10 ug/ml to 0.1 ng/ml in 10-fold dilutions. ADCC assay: 50 ul of R347, 50 ul of target cells and 50 ul of effector cells were added in duplicate in row A of a 96-well round bottom plate (E:T ratio=2.5:1). 50 ul of MEDI-524, 50 ul of target cells and 50 ul of effector cells were added in duplicate in row B of the 96-well round bottom plate (E:T ratio=2.5:1). 50 ul of Medi524-3M, 50 ul of target cells and 50 ul of effector cells were added in duplicate in row C of the 96-well round bottom plate (E:T ratio=2.5:1). Row D had the following control groups in duplicate:

Tonly—50 ul Target cells+100 ul RP-5
Tmax—50 ul Target cells+80 ul RP-5 (+20 ul Lysis buffer)
T+E—50 ul Target cells+50 ul Effector cells+50 ul RP-5

### Media—150 ul RP-5

Detergent—130 ul RP-5 (+20 ul Lysis buffer)
Plates were spun at 120 g for 3 mins, then incubated at 37° C./5% CO₂ for 4 hrs. 45 mins prior to the end of the 4 hr incubation, 20 ul of lysis buffer (from LDH kit) was added to the plate wells with Tmax and Detergent (see above). After 4 hrs of incubation, plates were spun at 120 g for 5 mins. For performing the LDH release assay (see below), 50 ul from each well was transferred into a new flat-bottom 96-well plate.

LDH release assay: (Promega®, #G1780, Non-radioactive cytotoxicity assay). Thawed the assay buffer from Promega kit to RT. Added 12 mls of assay buffer to one vial substrate mix from the kit, protected from light, and used immediately (for one whole plate). Added 50 ul of substrate solution to each well (in the 96 well flat bottom plate which already has 50 ul of samples) and incubated 15-20 mins in the dark at RT. Added 50 ul of stop solution from the kit to each well, popped any bubbles and read the OD at 490 nm within one hour.

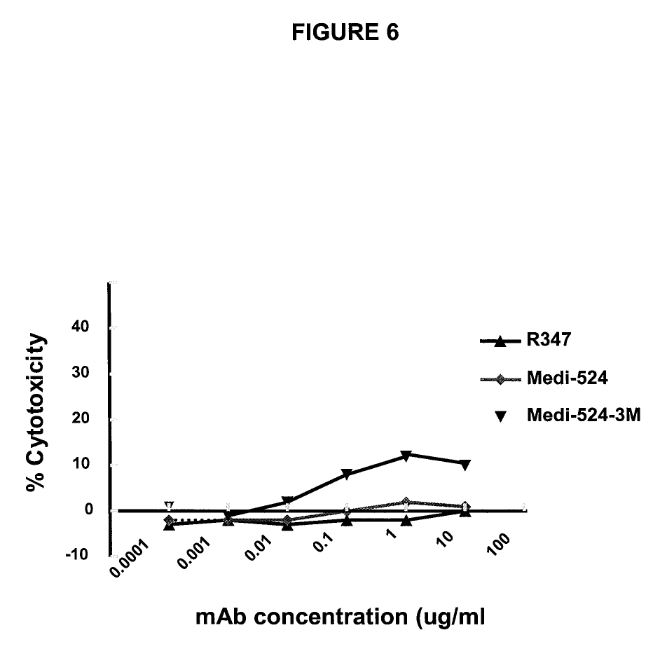

The results of the assay are shown in FIG. 6. MEDI-524 3M is engineered for enhanced ADCC effector function, as compared to MEDI-524. As a result, MEDI-524 3M demonstrated more ADCC cytotoxicity than MEDI-524, (approximately 10-12% cytotoxicity).

### Example 5

### Therapeutic Efficacy of MEDI-524 TM

Treatment efficacy was tested in the following experiment using modified MEDI-524 antibodies, MEDI-524 3M and MEDI-524 TM (having amino acid mutations of 234F, 235E, 331S as in Kabat numbering) to see if such Fc region modifications could further increase the effectiveness of MEDI-524.

MEDI-524 was diluted in sterile saline from a stock concentration of 100 mg/ml. For each study, juvenile cotton rats (Sigmodon hispidus, average weight 100 g from Virion Systems, Inc. Rockville, Md.) were separated into groups of four cotton rats each. Animals were dosed 0.1 mL of test article at different time points (24 hrs prior infection and 24 or 72 hrs post infection) by intraperitoneal injection, one group of cotton rats for each dose of motavizumab or control antibody (MEDI-507). Twenty four hours later, animals were anesthetized with isofluorane and challenged by intranasal instillation of 1×10⁵ pfu/animal RSV A2 (from ATCC). Four days later, animals were sacrificed by carbon dioxide asphyxiation, their lungs were surgically removed, bisected and snap frozen in liquid nitrogen. Nasal tissues were excised using a sterile scalpel and also frozen in liquid nitrogen. Lungs were individually homogenized in 20 parts (weight/volume) HBSS (catalog #14175, Invitrogen, Carlsbad, Calif.) using glass tissue homogenizers, noses were homogenized, using 10 parts (weight/volume) HBSS, sterile quartz sand and mortar and pestle. The resultant suspensions were centrifuged at 770×g for 10 minutes, and the supernatants were collected and stored at −80° C. until analysis of viral titers by plaque titration.

Plaque reduction assay (PRA): F Lung homogenate samples were diluted 1:10 and 1:100 in HBSS, and 50 uL aliquots of neat, 1:10 and 1:100 dilutions were added to duplicate wells of HEp-2 cells (ATCC #CCL-23) in 24-well plates. After 1 hour incubation at 37° C., the inoculum was replaced with culture medium containing 1% methylcellulose (#M0512-5000, Sigma-Aldrich, Inc., St. Louis, Mo.) and the cells were returned to a 37° C. incubator. Four days later the overlay was removed and the cells were fixed and stained with 0.1% crystal violet in 5% glutaraldehyde for 30 minutes, washed, air dried, and the plaques were counted. The limit of detection for this assay was 200 PFU/gram of tissue. Samples with a virus titer below the limit of detection were <200 PFU/gm=log₁₀ of 2.3.

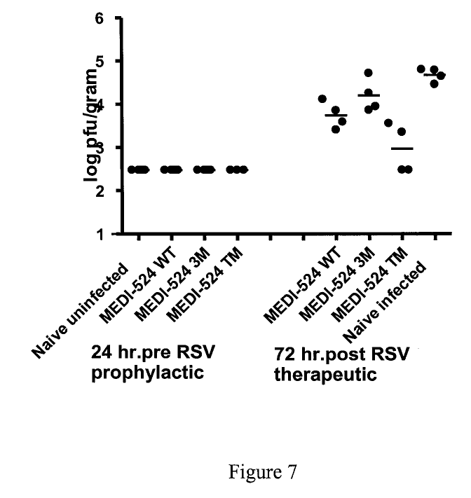

The results are shown in FIG. 7. Treatment with MEDI-524 TM demonstrates an apparent efficacy of lowering RSV viral titers as compared to MEDI-524.

### Example 6

### Cotton Rat Prophylaxis

To determine the ability of any one of the antibodies described herein or their fragments to treat respiratory tract RSV infection in cotton rats when administered by and intravenous (IV) route and to correlate the serum concentration of said antibody or fragment with a reduction in lung RSV titer. The example below uses SYNAGIS®, but can be applied to any of the antibodies described herein or their fragments.

### Materials & Methods

SYNAGIS® lot L94H048 was used for studies III-47 and III-47A. SYNAGIS® lot L95 K016 was used for study III-58. Bovine serum albumin (BSA) (fraction V, Sigma Chemicals). RSV-Long (A subtype) was propagated in Hep-2 cells.

On day 0, to groups of cotton rats (Sigmodon hispidis, average weight 100 g) were administered SYNAGIS®, RSV-IGIV or BSA was administered by intramuscular injection. Twenty-four hours post administration, the animals were bled and infected intranasally with 105 pfu of RSV. Twenty-four hours later, the animals were bled and infected intranasally with 10⁵ PFU or RSV (Long Strain). Four days after the infection, animals were sacrificed, and their lung tissue was harvested and pulmonary virus titers were determined by plaque titration. For studies III-47 and III-47A, the doses of monoclonal antibody (“MAb”) consisted of 0.31, 0.63, 1.25, 2.5, 5.5 and 10 mg/kg (body weight). For studies III-58, the doses of MAb consisted of 0.63, 1.25, 2.5, 5.5 and 10 mg/kg (body weight). In all three studies bovine serum albumin (BSA) 10 mg/kg was used as a negative control. Human antibody concentrations in the serum at the time of challenge are determined using a sandwich ELISA.

### Results

The results of the individual experiments are presented in Tables 2-5. The results of all of the experiments combined are shown in Table 5. All three studies show a significant reduction of pulmonary virus titers in animals treated with SYNAGIS®. A clear dose-response effect was observed in the animals. The combined data indicated that a dose of 2.5 mg/kg results in a greater than 99% reduction in lung RSV titer. The mean serum concentration of SYNAGIS® for this dose at the time of viral challenge was 28.6 mg/ml.

TABLE 2
EXPERIMENT III-47

| | | | | Lung Viral |
|---|---|---|---|---|
| | | | Mean ± Std | Titer |
| | | | Error | Geometric |
| | Number | | Concentration | Mean ± |
| | of | | of Human IgG | Std Error |
| Compound | Animals | Dose | (mg/ml) | (log10 pfu/gm) |
| BSA | 4 | | 0 | 1.4 × 10^{5±}1.7 |
| SYNAGIS ® | 3 | 0.312 mg/kg | 3.83 ± 1.1 | 2.1 × 10^{4±}2.1 |
| SYNAGIS ® | 3 | 0.625 mg/kg | 5.27 ± 0.37 | 7.7 × 10^{4±}1.6 |
| SYNAGIS ® | 4 | 1.25 mg/kg | 9.15 ± 0.16 | 3.4 × 10^{4±}1.3 |
| SYNAGIS ® | 3 | 2.50 mg/kg | 23.4 ± 2.8 | 1.4 × 10^{3±}1.7 |
| SYNAGIS ® | 2 | 5.0 mg/kg | 42.4 ± 13.4 | 4.6 × 10^{2±}4.6 |
| SYNAGIS ® | 4 | 10.0 mg/kg | 141.1 ± 14.4 | 1.0 × 10^{2±}1.0 |

TABLE 3
EXPERIMENT III-47A

| | | | Mean ± Std | Lung Viral |
|---|---|---|---|---|
| | | | Error | Titer |
| | | | Concentration | Geometric |
| | Number | | of | Mean ± |
| | of | | Human IgG | Std Error |
| Compound | Animals | Dose | (mg/ml) | (log10 pfu/gm) |
| BSA | 4 | | 0 | 1.9 × 10^{5±}1.2 |
| SYNAGIS ® | 4 | 0.312 mg/kg | 1.8 ± 0.12 | 8.5 × 10^{4±}1.2 |
| SYNAGIS ® | 4 | 0.625 mg/kg | 4.0 ± 0.19 | 5.0 × 10^{4±}1.6 |
| SYNAGIS ® | 4 | 1.25 mg/kg | 11.8 ± 0.68 | 1.9 × 10^{3±}1.4 |
| SYNAGIS ® | 4 | 2.50 mg/kg | 18.9 ± 2.0 | 5.3 × 10^{3±}1.6 |
| SYNAGIS ® | 3 | 5.0 mg/kg | 55.6 ± 2.3 | 1.6 × 10^{2±}1.3 |
| SYNAGIS ® | 4 | 10.0 mg/kg | 109.7 ± 5.22 | 1.0 × 10^{2±}1.0 |

TABLE 4
EXPERIMENT III-58

| | | | | Lung |
|---|---|---|---|---|
| | | | Mean ± Std | Viral Titer |
| | | | Error | Geometric |
| | Number | | Concentration | Mean ± |
| | of | | of Human IgG | Std Error |
| Compound | Animals | Dose | (mg/ml) | (log10 pfu/gm) |
| BSA | 4 | | 0 | 1.1 × 10^{5±}1.2 |
| SYNAGIS ® | 4 | 0.625 mg/kg | 5.78 ± 0.32 | 1.6 × 10^{4±}1.2 |
| SYNAGIS ® | 4 | 1.25 mg/kg | 9.82 ± 0.23 | 1.6 × 10^{3±}1.3 |
| SYNAGIS ® | 4 | 2.50 mg/kg | 34.1 ± 2.11 | 4.3 × 10^{2±}1.6 |
| SYNAGIS ® | 3 | 5.0 mg/kg | 58.3 ± 4.48 | 1.0 × 10^{2±}1.0 |
| SYNAGIS ® | 4 | 10.0 mg/kg | 111.5 ± 5.04 | 1.0 × 10^{2±}1.0 |

TABLE 5
III-47, III-47A and III-58 COMBINED

| | | | | Lung |
|---|---|---|---|---|
| | | | Mean ± Std | Viral Titer |
| | | | Error | Geometric |
| | Number | | Concentration | Mean ± |
| | of | | of Human IgG | Std Error |
| Compound | Animals | Dose | (mg/ml) | (log10 pfu/gm) |
| BSA | 18 | | 0 | 1.3 × 10^{5±}1.2 |
| SYNAGIS ® | 7 | 0.312 mg/kg | 2.67 ± 0.60 | 4.6 × 10^{4±}1.5 |
| SYNAGIS ® | 17 | 0.625 mg/kg | 5.27 ± 0.27 | 2.7 × 10^{4±}1.3 |
| SYNAGIS ® | 18 | 1.25 mg/kg | 10.1 ± 0.29 | 3.3 × 10^{3±}1.4 |
| SYNAGIS ® | 17 | 2.50 mg/kg | 28.6 ± 2.15 | 9.6 × 10^{2±}1.5 |
| SYNAGIS ® | 15 | 5.0 mg/kg | 55.6 ± 3.43 | 1.3 × 10^{2±}1.2 |
| SYNAGIS ® | 18 | 10.0 mg/kg | 117.6 ± 5.09 | 1.0 × 10^{2±}1.0 |

### Example 7

### Measuring PD-L1 Expression after Motavizumab (MEDI-524) Treatment of RSV-Infected A549 Cells

On day 1, three, 12 well plates were seeded with A549 cells to P15 at 4×10⁵ cells/well. On day 3, the A549 cells were infected with RSVA at a multiplicity of infection (MOI) of 1.0. After 1 hr of infection, a control, non-relevant antibody, MEDI-507 (50706F-0016, 10.2 mg/ml) or the experimental antibody MEDI-524 (52405G-0964, 10.2 mg/ml) at 10 ug/ml to appropriate wells. After 6 hrs and 12 hrs of infection, either MEDI-507 or MEDI-524 antibodies were added to appropriate wells.

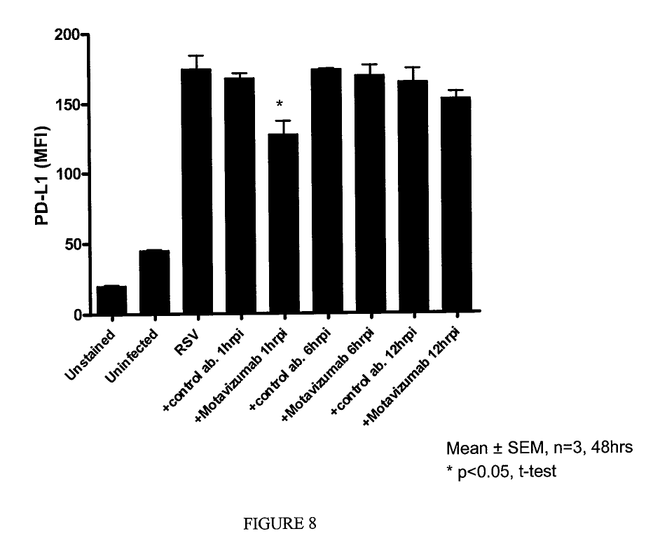

After 48 hours of infection, the A549 cells (numbering at approximately 500,000) were stained with PD-L1 PE (eBioscience®, cat #12-5983-71). Cells acquired on LSR II green (20,000 events per sample). Results were quantitated and graphed (see FIG. 8).

### Example 8

### Measuring ICAM-1 Expression after Motavizumab (MEDI-524) Treatment of RSV-Infected A549 Cells

On day 1, two, 12 well plates were seeded with A549 cells P14 at 3×10⁵ cells/well in 2 mls. On day 3, confluent A549 cells were infected with RSV A 1×10⁸ pfu/ml) at a MOI of 1.0.

After 1 hr, 6 hrs and 12 hrs post RSV infection, added a control, non-relevant antibody, MEDI-507 (50706F-0016; Lot 06AZ03 10.2 mg/ml) and the experimental antibody motavizumab or MEDI-524 (Lot 05M02-76; fill date 2 Dec. 2005 102 mg/ml) at 10 ug/ml to appropriate wells. Incubated cultures for 48 hrs.

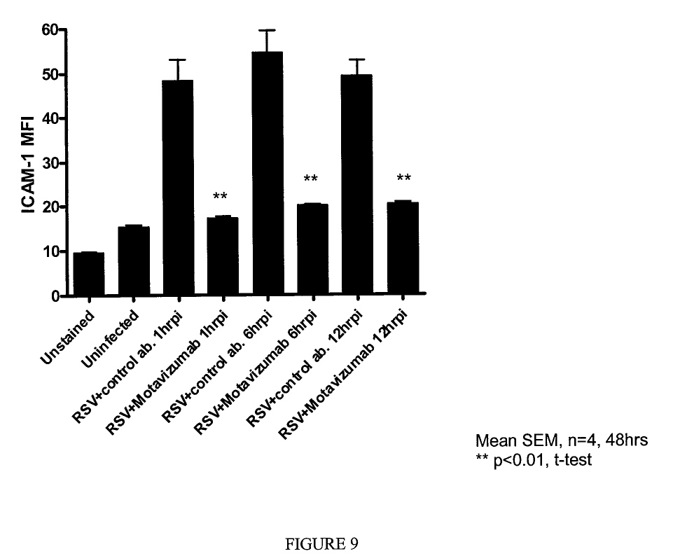

After the incubation period, the infected A549 cells (at an approximate cell number of 500,000) were stained with ICAM-1 APC (cat #559771, BD®). Cells acquired on LSR II green (50,000 events per sample). Results were quantitated and graphed (see FIG. 9).

### Example 9

### Measuring Cell Apoptosis after Motavizumab (MEDI-524) Treatment of RSV-Infected A549 Cells

On day 1, two, 12 well plates were seeded with A549 cells P26 at 3.5×10⁵ cells/well in 2 mls. On day 3, confluent A549 cells were infected with RSV A at a MOI of 1.0.

Motavizumab or MEDI-524 (Lot 05M02-76; fill date 2 Dec. 2005 102 mg/ml) at 10 ug/ml was added at timepoints 1 hr, 6 hrs and 12 hrs post-RSV infection to appropriate wells. The cell cultures were incubated for 72 hrs.

Adherent cells were dissociated and pooled with floating cells, pelleted by centrifugation and resuspended in 1 ml of media (˜1×10⁶ cells). Approximately 20,000 cells/well were added to a 96 well plate (white-walled, clear bottom) in a volume of 100 ul.

A cell titer-glo assay (cat #G7571, Luminescent cell viability kit, Promega®) and Caspase-glo 3/7 assay (cat #G8091, Promega®) reagents were added to appropriate wells (100 ul/well).

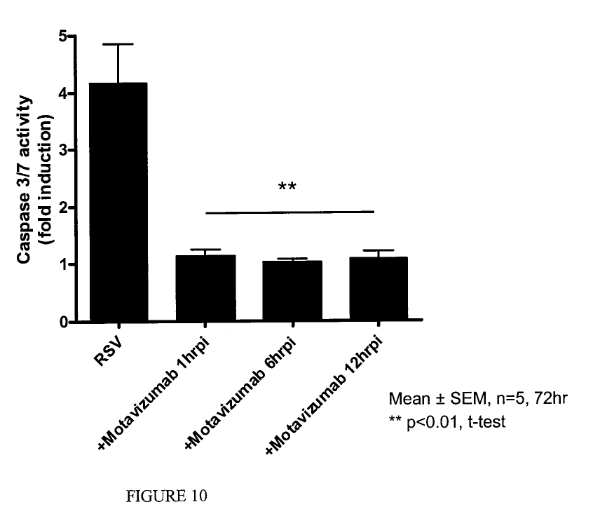

Incubated at RT in the dark for 1 hr. Luminescence was measured using the SpectraMax M5 microplate reader (Molecular Devices®). Results were quantitated and graphed (see FIG. 10).

### Example 10

### Measuring % Floating Cells after Motavizumab (MEDI-524) Treatment of RSV-Infected A549 Cells

On day 1, two, 12 well plates were seeded with A549 cells P26 at 3.5×10⁵ cells/well in 2 mls. On day 3, confluent A549 cells were infected with RSV A at a MOI of 1.0.

Motavizumab or MEDI-524 (Lot 05M02-76; fill date 2 Dec. 2005 102 mg/ml) at 10 ug/ml was added at timepoints 1 hr, 6 hrs and 12 hrs post-RSV infection to appropriate wells. The cell cultures were incubated for 72 hrs.

Cells floating in the cell culture supernatants were collected and counted. Adherent cells were dissociated and counted separately as well, as follows:

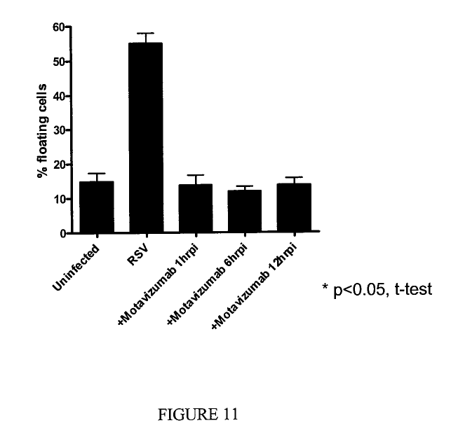

% floating cells=(Number of floating cells/Total number of cells)×100 (Total number of cells=Floating cell count+Adherent cell count). Results were quantitated and graphed (see FIG. 11).

### Example 11

### Measuring RSV Release in Cell Culture Supernatants after Motavizumab (MEDI-524) Treatment of RSV-Infected HEp-2 and A549 Cells

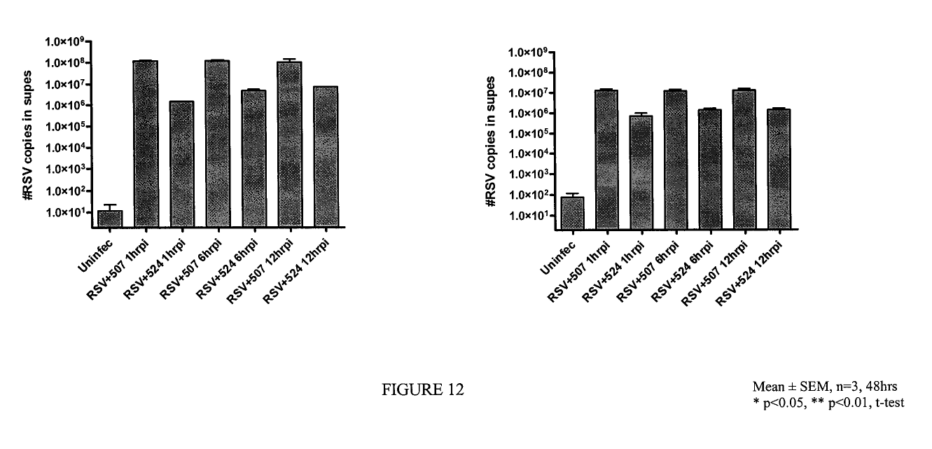

The cell culture supernatants collected above, in Example 10 for A549 cells and repeated for HEp-2 cells were analyzed to quantitate the amount of RSV released into the supernatant as a measure of live, RSV replication occurring in the cell cultures. See FIG. 12 for results.

### Example 12

### Primary Lung Epithelial Cell Air-Liquid Interface System

This planned experiment will closely mimic the polarity of lung epithelial cells at an air-liquid interface (ALI). See, Zhang et al., Respiratory Syncytial Virus infection of human airway epithelial cells is polarized, specific to ciliated cells and without obvious cytopathology, J Virol Vol 76: 5654-5666, (2002); and Mellow et al., The Effect of RSV on chemokine release by differentiated airway epithelium, Expt. Lung Research 30: 43-57, (2004).

In well plates, infect primary lung epithelial cells that are cultured and maintained at an air-liquid interface (ALI) with either laboratory strains of RSV A or RSV obtained from clinical isolates from patients at a multiplicity of infection (MOI) of 1.0, 0.1 and 0.01 and add motavizumab (MEDI-524) at 6-12 hrs, 24 hrs and 48 hrs post RSV infection respectively. These cultures will be incubated for between 24-48 hrs, 48-72 hrs and 72-96 hrs respectively. The RSV replication, cytokine secretion (protein) and cytokine gene expression (IL-6, IL-8, TNF-a, MIP-1α and RANTES), cell surface immune markers (PD-L1, ICAM-1, TLR4) and cellular apoptosis will be evaluated according to methods described herein. This experimental design will be compared to a prophylactic scenario in which primary lung epithelial cells, grown in an ALI, will be pre-treated with motavizumab (MEDI-524) for approximately 1 hr pre-infection. Then, the epithelial cells will be infected with either laboratory RSV A or RSV obtained from clinical isolates from patients. The resulting prophylactic outcome will be compared to the therapeutic application described above.

### Example 13

### Clinical Trials

Antibodies of the invention or fragments thereof tested in in vitro assays and animal models may be further evaluated for safety, tolerance and pharmacokinetics in groups of normal healthy adult volunteers. The volunteers are administered intramuscularly, intravenously or by a pulmonary delivery system a single dose of 0.5 mg/kg, 3 mg/kg, 5 mg/kg, 10 mg/kg or 15 mg/kg of an antibody or fragment thereof which immunospecifically binds to a RSV antigen. Each volunteer is monitored at least 24 hours prior to receiving the single dose of the antibody or fragment thereof and each volunteer will be monitored for at least 48 hours after receiving the dose at a clinical site. Then volunteers are monitored as outpatients on days 3, 7, 14, 21, 28, 35, 42, 49, and 56 postdose.

Blood samples are collected via an indwelling catheter or direct venipuncture using 10 ml red-top Vacutainer tubes at the following intervals: (1) prior to administering the dose of the antibody or antibody fragment; (2) during the administration of the dose of the antibody or antibody fragment; (3) 5 minutes, 10 minutes, 15 minutes, 20 minutes, 30 minutes, 1 hour, 2 hours, 4 hours, 8 hours, 12 hours, 24 hours, and 48 hours after administering the dose of the antibody or antibody fragment; and (4) 3 days, 7 days 14 days, 21 days, 28 days, 35 days, 42 days, 49 days, and 56 days after administering the dose of the antibody or antibody fragment. Samples are allowed to clot at room temperature and serum will be collected after centrifugation.

The antibody or antibody fragment is partially purified from the serum samples and the amount of antibody or antibody fragment in the samples will be quantitated by ELISA. Briefly, the ELISA consists of coating microtiter plates overnight at 4° C. with an antibody that recognizes the antibody or antibody fragment administered to the volunteer. The plates are then blocked for approximately 30 minutes at room temperate with PBS-Tween-0.5% BSA. Standard curves are constructed using purified antibody or antibody fragment, not administered to a volunteer. Samples are diluted in PBS-Tween-BSA. The samples and standards are incubated for approximately 1 hour at room temperature. Next, the bound antibody is treated with a labeled antibody (e.g., horseradish peroxidase conjugated goat-anti-human IgG) for approximately 1 hour at room temperature. Binding of the labeled antibody is detected, e.g., by a spectrophotometer.

The concentration of antibody or antibody fragment levels in the serum of volunteers are corrected by subtracting the predose serum level (background level) from the serum levels at each collection interval after administration of the dose. For each volunteer the pharmacokinetic parameters are computed according to the model-independent approach (Gibaldi et al., eds., 1982, Pharmacokinetics, 2^{nd} edition, Marcel Dekker, New York) from the corrected serum antibody or antibody fragment concentrations.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A modified antibody that immunospecifically binds to a RSV F antigen, said modified antibody comprising three variable heavy complementarity determining regions (VH CDRs) and three variable light CDRs (VL CDRs) having an amino acid sequence of a VH CDR 1, 2 and 3 and VL CDR 1, 2 and 3 of A4B4L1FR-S28R, of A4B4-F52S, of AFFF, of P12f2, of P12f4, of P11d4, of A1e9, of A12a6, of A13c4, of A17d4, of A4B4, of A8c7, of IX-493L1FR, of H3-3F4, of M3H9, of Y10H6, of DG, of AFFF(1), of 6H8, of L1-7E5, of L2-15B10, of A13a11, of A1h5, or of A4B4(1), as shown in Table 1, wherein said modified antibody has a modified human IgG Fc domain comprising one or more amino acid substitutions relative to a wild-type human IgG Fc domain, wherein said amino acid substitutions results in said modified antibody comprising an altered binding affinity for one or more Fc receptors as compared to a wild-type antibody without said amino acid substitutions.

2. (canceled)

3. The modified antibody of claim 1, wherein the modified IgG Fc domain comprises an amino acid substitution at amino acid residue 332E, as numbered by the EU index as set forth in Kabat.

4. The modified antibody of claim 3, wherein the modified IgG Fc domain further comprises amino acid substitutions at amino acid residues 239D and 330L, as numbered by the EU index as set forth in Kabat.

5. The modified antibody of claim 1, wherein the one or more amino acid substitutions is selected from the group consisting of: 234E, 235R, 235A, 235W, 235P, 235V, 235Y, 236E, 239D, 265L, 269S, 269G, 298I, 298T, 298F, 327N, 327G, 327W, 328S, 328V, 329H, 329Q, 330K, 330V, 330G, 330Y, 330T, 330L, 330I, 330R, 330C, 332E, 332H, 332S, 332W, 332F, 332D, and 332Y, wherein the numbering system is that of the EU index as set forth in Kabat.

6. The modified antibody of claim 1, wherein the modified IgG Fc domain comprises an amino acid substitution at amino acid residue 331S, as numbered by the EU index as set forth in Kabat.

7. The modified antibody of claim 6, wherein the modified IgG Fc domain further comprises amino acid substitutions at amino acid residues 234F and 235E, as numbered by the EU index as set forth in Kabat.

8. The modified antibody of claim 1, wherein the one or more amino acid substitutions is selected from the group consisting of: 233P, 234V, 235A, 265A, 327G, and 330S, wherein the numbering system is that of the EU index as set forth in Kabat.

9. (canceled)

10. The modified antibody of claim 1, wherein said one or more amino acid substitutions are at one or more of amino acid residues 251, 252, 254, 255, 256, 308, 309, 311, 312, 314, 385, 386, 387, 389, 428, 433, 434 and 436, wherein said additional amino acid substitutions are substitution with leucine position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, substitution with histidine, tyrosine, arginine or threonine at position 436, wherein the numbering system is that of the EU index as set forth in Kabat.

11. (canceled)

12. The modified antibody of claim 10, wherein said one or more amino acid substitutions are substitutions with tyrosine at position 252, threonine at position 254 and glutamate at 256, wherein the numbering system is that of the EU index as set forth in Kabat.

13. A composition comprising the modified antibody of claim 12 in a sterile carrier.

14. A method of treating a human patient infected with RSV, the method comprising administering to said patient in need thereof a therapeutically effective amount of the composition of claim 12.

15. The method of claim 14, wherein the therapeutically effective amount is selected from the group consisting of about 30 mg/kg, about 25 mg/kg, about 20 mg/kg, about 15 mg/kg, about 10 mg/kg, about 5 mg/kg, about 3 mg/kg, about 1.5 mg/kg, about 1 mg/kg, about 0.75 mg/kg, about 0.5 mg/kg, about 0.25 mg/kg, about 0.1 mg/kg, about 0.05 mg/kg, and about 0.025 mg/kg.

16. The method of claim 14, wherein said human patient has had a bone marrow transplant, has cystic fibrosis, has bronchopulmonary dysplasia, has congenital heart disease, has chronic obstructive pulmonary disease (COPD), has congenital immunodeficiency or has acquired immunodeficiency.

17. The method of claim 14, wherein said human patient is an infant, an infant born prematurely, an infant who has been hospitalized for a RSV infection, or an infant predisposed to asthma and/or reactive airway disease (RAD), and/or wheezing or a child aged 0 to 5 years.

18. The method of claim 14, wherein the human patient is an elderly human, or is living in a nursing home.

19. The method of claim 14, wherein said composition is administered to said human patient by intranasal delivery, intramuscular delivery, intradermal delivery, intraperitoneal delivery, intravenous delivery, subcutaneous delivery, oral delivery, pulmonary delivery or combinations thereof.

20. (canceled)

21. The method of claim 14, wherein said therapeutic administration of said modified antibody inhibits or downregulates RSV replication in said human patient by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% as compared to a control in which no therapeutic administration of said modified antibody is performed, as measured by viral shedding.

22. The method of claim 14, wherein said therapeutic administration of said modified antibody decreases serum levels of cytokines in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.

23. The method of claim 14, wherein said therapeutic administration of said modified antibody decreases serum levels of chemokine release in said human patient by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to a control in which no therapeutic administration of said modified antibody is performed, as measured by a bioassay.

24. (canceled)

25. (canceled)
